SEQUENCE LISTING (30 sequences)

SEQ ID NO: 1 - DNA, 2577 bp, Human Immunodeficiency Virus-1
  CDS (10)...(2562)
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggatgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggatgacctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggatggggctgccaacagggagaccaagctgggcaaggctggctatgtgaccaacaggggcaggcagaaggtggtgaccctgactgacaccaccaaccagaagactgagctccaggccatctacctggccctccaggactctggcctggaggtgaacattgtgactgactcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggactgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgacaatggctccaacttcactggggccacagtgagggctgcctgctggtgggctggcatcaagcaggagtttggcatcccctacaacccccagtcccagggggtggtggagtccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 2 - protein, 850 aa, Human Immunodeficiency Virus-1
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 3 - DNA, 2577 bp, Human Immunodeficiency Virus-1
  CDS (10)...(2562)
agatctaccatggcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 4 - protein, 850 aa, Human Immunodeficiency Virus-1
MAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 5 - DNA, 2650 bp, Human Immunodeficiency Virus-1
  CDS (8)...(2635)
gatcaccatggatgcaatgaagagagggctctgctgtgtgctgctgctgtgtggagcagtcttcgtttcgcccagcgagatctccgcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggatgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggatgacctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggatggggctgccaacagggagaccaagctgggcaaggctggctatgtgaccaacaggggcaggcagaaggtggtgaccctgactgacaccaccaaccagaagactgagctccaggccatctacctggccctccaggactctggcctggaggtgaacattgtgactgactcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggactgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgacaatggctccaacttcactggggccacagtgagggctgcctgctggtgggctggcatcaagcaggagtttggcatcccctacaacccccagtcccagggggtggtggagtccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 6 - protein, 875 aa, Human Immunodeficiency Virus-1
MDAMKRGLCCVLLLCGAVFVSPSEISAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 7 - DNA, 2650 bp, Human Immunodeficiency Virus-1
  CDS (8)...(2635)
gatcaccatggatgcaatgaagagagggctctgctgtgtgctgctgctgtgtggagcagtcttcgtttcgcccagcgagatctccgcccccatctcccccattgagactgtgcctgtgaagctgaagcctggcatggatggccccaaggtgaagcagtggcccctgactgaggagaagatcaaggccctggtggaaatctgcactgagatggagaaggagggcaaaatctccaagattggccccgagaacccctacaacacccctgtgtttgccatcaagaagaaggactccaccaagtggaggaagctggtggacttcagggagctgaacaagaggacccaggacttctgggaggtgcagctgggcatcccccaccccgctggcctgaagaagaagaagtctgtgactgtgctggctgtgggggatgcctacttctctgtgcccctggatgaggacttcaggaagtacactgccttcaccatcccctccatcaacaatgagacccctggcatcaggtaccagtacaatgtgctgccccagggctggaagggctcccctgccatcttccagtcctccatgaccaagatcctggagcccttcaggaagcagaaccctgacattgtgatctaccagtacatggctgccctgtatgtgggctctgacctggagattgggcagcacaggaccaagattgaggagctgaggcagcacctgctgaggtggggcctgaccacccctgacaagaagcaccagaaggagccccccttcctgtggatgggctatgagctgcaccccgacaagtggactgtgcagcccattgtgctgcctgagaaggactcctggactgtgaatgacatccagaagctggtgggcaagctgaactgggcctcccaaatctaccctggcatcaaggtgaggcagctgtgcaagctgctgaggggcaccaaggccctgactgaggtgatccccctgactgaggaggctgagctggagctggctgagaacagggagatcctgaaggagcctgtgcatggggtgtactatgacccctccaaggacctgattgctgagatccagaagcagggccagggccagtggacctaccaaatctaccaggagcccttcaagaacctgaagactggcaagtatgccaggatgaggggggcccacaccaatgatgtgaagcagctgactgaggctgtgcagaagatcaccactgagtccattgtgatctggggcaagacccccaagttcaagctgcccatccagaaggagacctgggagacctggtggactgagtactggcaggccacctggatccctgagtgggagtttgtgaacaccccccccctggtgaagctgtggtaccagctggagaaggagcccattgtgggggctgagaccttctatgtggctggggctgccaacagggagaccaagctgggcaaggctggctatgtgaccaacaggggcaggcagaaggtggtgaccctgactgacaccaccaaccagaagactgccctccaggccatctacctggccctccaggactctggcctggaggtgaacattgtgactgcctcccagtatgccctgggcatcatccaggcccagcctgatcagtctgagtctgagctggtgaaccagatcattgagcagctgatcaagaaggagaaggtgtacctggcctgggtgcctgcccacaagggcattgggggcaatgagcaggtggacaagctggtgtctgctggcatcaggaaggtgctgttcctggatggcattgacaaggcccaggatgagcatgagaagtaccactccaactggagggctatggcctctgacttcaacctgccccctgtggtggctaaggagattgtggcctcctgtgacaagtgccagctgaagggggaggccatgcatgggcaggtggactgctcccctggcatctggcagctggcctgcacccacctggagggcaaggtgatcctggtggctgtgcatgtggcctccggctacattgaggctgaggtgatccctgctgagacaggccaggagactgcctacttcctgctgaagctggctggcaggtggcctgtgaagaccatccacactgccaatggctccaacttcactggggccacagtgagggctgcctgctggtgggctggcatcaagcaggagtttggcatcccctacaacccccagtcccagggggtggtggcctccatgaacaaggagctgaagaagatcattgggcaggtgagggaccaggctgagcacctgaagacagctgtgcagatggctgtgttcatccacaacttcaagaggaaggggggcatcgggggctactccgctggggagaggattgtggacatcattgccacagacatccagaccaaggagctccagaagcagatcaccaagatccagaacttcagggtgtactacagggactccaggaaccccctgtggaagggccctgccaagctgctgtggaagggggagggggctgtggtgatccaggacaactctgacatcaaggtggtgcccaggaggaaggccaagatcatcagggactatggcaagcagatggctggggatgactgtgtggcctccaggcaggatgaggactaaagcccgggcagatct

SEQ ID NO: 8 - protein, 875 aa, Human Immunodeficiency Virus-1
MDAMKRGLCCVLLLCGAVFVSPSEISAPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLAVGDAYFSVPLDEDFRKYTAFTIPSINNETPGIRYQYNVLPQGWKGSPAIFQSSMTKILEPFRKQNPDIVIYQYMAALYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVAGAANRETKLGKAGYVTNRGRQKVVTLTDTTNQKTALQAIYLALQDSGLEVNIVTASQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLACTHLEGKVILVAVHVASGYIEAEVIPAETGQETAYFLLKLAGRWPVKTIHTANGSNFTGATVRAACWWAGIKQEFGIPYNPQSQGVVASMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 9 - DNA, 4945 bp, E. coli (V1Jns-tpa)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatcaccatggatgcaatgaagagagggctctgctgtgtgctgctgctgtgtggagcagtcttcgtttcgcccagcgagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 23 bp, Artificial Sequence
  oligonucleotide
ctatataagcagagctcgtttag

SEQ ID NO: 11 - DNA, 30 bp, Artificial Sequence
  oligonucleotide
gtagcaaagatctaaggacggtgactgcag

SEQ ID NO: 12 - DNA, 39 bp, Artificial Sequence
  oligonucleotide
gtatgtgtctgaaaatgagcgtggagattgggctcgcac

SEQ ID NO: 13 - DNA, 39 bp, Artificial Sequence
  oligonucleotide
gtgcgagcccaatctccacgctcattttcagacacatac

SEQ ID NO: 14 - DNA, 4432 bp, E. coli (V1J plasmid)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 4864 bp, E. coli (V1Jneo plasmid)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 4867 bp, E. coli (V1Jns plasmid)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 75 bp, Artificial Sequence
  oligonucleotide
gatcaccatggatgcaatgaagagagggctctgtgtgctgctgctgtgtggagcagtcttcgtttcgcccagcga

SEQ ID NO: 18 - DNA, 78 bp, Artificial Sequence
  oligonucleotide
gatctcgctgggcgaaacgaagactgctccacacagcagcagcacacagcagagccctctcttcattgcatccatggt

SEQ ID NO: 19 - DNA, 33 bp, Artificial Sequence
  oligonucleotide
ggtacaaatattggctattggccattgcatacg

SEQ ID NO: 20 - DNA, 36 bp, Artificial Sequence
  oligonucleotide
ccacatctcgaggaaccgggtcaattcttcagcacc

SEQ ID NO: 21 - DNA, 38 bp, Artificial Sequence
  oligonucleotide
ggtacagatatcggaaagccacgttgtgtctcaaaatc

SEQ ID NO: 22 - DNA, 36 bp, Artificial Sequence
  oligonucleotide
cacatggatccgtaatgctctgccagtgttacaacc

SEQ ID NO: 23 - DNA, 39 bp, Artificial Sequence
  oligonucleotide
ggtacatgatcacgtagaaaagatcaaaggatcttcttg

SEQ ID NO: 24 - DNA, 35 bp, Artificial Sequence
  oligonucleotide
ccacatgtcgacccgtaaaaaggccgcgttgctgg

SEQ ID NO: 25 - DNA, 4864 bp, E. coli (V1R plasmid); identical to SEQ ID NO: 15

SEQ ID NO: 26 - DNA, 36 bp, Artificial Sequence
  oligonucleotide
ggtacaagatctccgcccccatctcccccattgaga

SEQ ID NO: 27 - DNA, 33 bp, Artificial Sequence
  oligonucleotide
ccacatagatctgcccgggctttagtcctcatc

SEQ ID NO: 28 - protein, 27 aa, Homo sapien
MDAMKRGLCCVLLLCGAVFVSPSEISS

SEQ ID NO: 29 - DNA, 45 bp, Artificial Sequence
  oligonucleotide
caggcgagatctaccatggcccccattagccctattgagactgta

SEQ ID NO: 30 - DNA, 48 bp, Artificial Sequence
  oligonucleotide
caggcgagatctgcccgggctttaatcctcatcctgtctacttgccac

(19) United States
(12) Patent Application Publication
(10) Pub. No.: US 2004/0063653 A1
(43) Pub. Date: Apr. 1, 2004

(54) Polynucleotide vaccines expressing codon optimized hiv-1 pol and modified hiv-1 pol

(76) Inventors: John W. Shiver, Chalfont, PA (US); Helen C. Perry, Lansdale, PA (US); Danilo R. Casimiro, Harleysville, PA (US); Tong-Ming Fu, Lansdale, PA (US)

Correspondence Address: MERCK AND CO INC, P O BOX 2000, RAHWAY, NJ, 070650907

(21) Appl. No.: 10/168,217

(22) PCT Filed: Dec. 21, 2000
(86) PCT No.: PCT/US00/34724

(51) Int. Cl. A61K 48/00; C07H 21/02

(52) U.S. Cl. 514/44; 536/23.1

(57) ABSTRACT

Pharmaceutical compositions which comprise HIV Pol DNA vaccines are disclosed, along with the production and use of these DNA vaccines. The pol-based DNA vaccines of the invention are administered directly introduced into living vertebrate tissue, preferably humans, and preferably express inactivated versions of the HIV Pol protein devoid of protease, reverse transcriptase activity, RNase H activity and integrase activity, inducing a cellular immune response which specifically recognizes human immunodeficiency virus-1 (HIV-1). The DNA molecules which comprise the open reading frame of these DNA vaccines are synthetic DNA molecules encoding codon optimized HIV-1 Pol and codon optimized inactive derivatives of optimized HIV-1 Pol, including DNA molecules which encode inactive Pol proteins which comprise an amino terminal leader peptide.

### CROSS-REFERENCE TO RELATED APPLICATIONS

This application claims the benefit, under 35 U.S.C. §119(e), of U.S. provisional application 60/171,542, filed Dec. 22, 1999.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to HIV Pol polynucleotide pharmaceutical products, as well as the production and use thereof which, when directly introduced into living vertebrate tissue, preferably a mammalian host such as a human or a non-human mammal of commercial or domestic veterinary importance, express the HIV Pol protein or biologically relevant portions thereof within the animal, inducing a cellular immune response which specifically recognizes human immunodeficiency virus-1 (HIV-1). The polynucleotides of the present invention are synthetic DNA molecules encoding codon optimized HIV-1 Pol and derivatives of optimized HIV-1 Pol, including constructs wherein protease, reverse transcriptase, RNAse H and integrase activity of HIV-1 Pol is inactivated. The polynucleotide vaccines of the present invention should offer a prophylactic advantage to previously uninfected individuals and/or provide a therapeutic effect by reducing viral load levels within an infected individual, thus prolonging the asymptomatic phase of HIV-1 infection.

### BACKGROUND OF THE INVENTION

Human Immunodeficiency Virus-1 (HIV-1) is the etiological agent of acquired human immune deficiency syndrome (AIDS) and related disorders. HIV-1 is an RNA virus of the Retroviridae family and exhibits the 5′LTR-gag-pol-env-LTR 3′ organization of all retroviruses. The integrated form of HIV-1, known as the provirus, is approximately 9.8 Kb in length. Each end of the viral genome contains flanking sequences known as long terminal repeats (LTRs). The HIV genes encode at least nine proteins and are divided into three classes; the major structural proteins (Gag, Pol, and Env), the regulatory proteins (Tat and Rev); and the accessory proteins (Vpu, Vpr, Vif and Nef).

The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6.

The pol gene encodes proteins necessary for virus replication; a reverse transcriptase, a protease, integrase and RNAse H. These viral proteins are expressed as a Gag-Pol fusion protein, a 160 kDa precursor protein which is generated via a ribosomal frame shifting. The viral encoded protease proteolytically cleaves the Pol polypeptide away from the Gag-Pol fusion and further cleaves the Pol polypeptide to the mature proteins which provide protease (Pro, P10), reverse transcriptase (RT, P50), integrase (IN, p31) and RNAse H (RNAse, p15) activities.

The nef gene encodes an early accessory HIV protein (Nef) which has been shown to possess several activities such as down regulating CD4 expression, disturbing T-cell activation and stimulating HIV infectivity.

The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells.

The tat gene encodes a long form and a short form of the Tat protein, a RNA binding protein which is a transcriptional transactivator essential for HIV-1 replication.

The rev gene encodes the 13 kDa Rev protein, a RNA binding protein. The Rev protein binds to a region of the viral RNA termed the Rev response element (RRE). The Rev protein is promotes transfer of unspliced viral RNA from the nucleus to the cytoplasm. The Rev protein is required for HIV late gene expression and in turn, HIV replication.

Gp120 binds to the CD4/chemokine receptor present on the surface of helper T-lymphocytes, macrophages and other target cells in addition to other co-receptor molecules. X4 (macrophage tropic) virus show tropism for CD4/CXCR4 complexes while a R5 (T-cell line tropic) virus interacts with a CD4/CCR5 receptor complex. After gp120 binds to CD4, gp41 mediates the fusion event responsible for virus entry. The virus fuses with and enters the target cell, followed by reverse transcription of its single stranded RNA genome into the double-stranded DNA via a RNA dependent DNA polymerase. The viral DNA, known as provirus, enters the cell nucleus, where the viral DNA directs the production of new viral RNA within the nucleus, expression of early and late HIV viral proteins, and subsequently the production and cellular release of new virus particles. Recent advances in the ability to detect viral load within the host shows that the primary infection results in an extremely high generation and tissue distribution of the virus, followed by a steady state level of virus (albeit through a continual viral production and turnover during this phase), leading ultimately to another burst of virus load which leads to the onset of clinical AIDS. Productively infected cells have a half life of several days, whereas chronically or latently infected cells have a 3-week half life, followed by non-productively infected cells which have a long half life (over 100 days) but do not significantly contribute to day to day viral loads seen throughout the course of disease.

Destruction of CD4 helper T lymphocytes, which are critical to immune defense, is a major cause of the progressive immune dysfunction that is the hallmark of HIV infection. The loss of CD4 T-cells seriously impairs the body's ability to fight most invaders, but it has a particularly severe impact on the defenses against viruses, fungi, parasites and certain bacteria, including mycobacteria.

Effective treatment regimens for HIV-1 infected individuals have become available recently. However, these drugs will not have a significant impact on the disease in many parts of the world and they will have a minimal impact in halting the spread of infection within the human population. As is true of many other infectious diseases, a significant epidemiologic impact on the spread of HIV-1 infection will only occur subsequent to the development and introduction of an effective vaccine. There are a number of factors that have contributed to the lack of successful vaccine development to date. As noted above, it is now apparent that in a chronically infected person there exists constant virus production in spite of the presence of anti-HIV-1 humoral and cellular immune responses and destruction of virally infected cells. As in the case of other infectious diseases, the outcome of disease is the result of a balance between the kinetics and the magnitude of the immune response and the pathogen replicative rate and accessibility to the immune response. Pre-existing immunity may be more successful with an acute infection than an evolving immune response can be with an established infection. A second factor is the considerable genetic variability of the virus. Although anti-HIV-1 antibodies exist that can neutralize HIV-1 infectivity in cell culture, these antibodies are generally virus isolate-specific in their activity. It has proven impossible to define serological groupings of HIV-1 using traditional methods. Rather, the virus seems to define a serological “continuum” so that individual neutralizing antibody responses, at best, are effective against only a handful of viral variants. Given this latter observation, it would be useful to identify immunogens and related delivery technologies that are likely to elicit anti-HIV-1 cellular immune responses. It is known that in order to generate CTL responses antigen must be synthesized within or introduced into cells, subsequently processed into small peptides by the proteasome complex, and translocated into the endoplasmic reticulum/Golgi complex secretory pathway for eventual association with major histocompatibility complex (MHC) class I proteins. CD8⁺ T lymphocytes recognize antigen in association with class I MHC via the T cell receptor (TCR) and the CD8 cell surface protein. Activation of naive CD8⁺ T cells into activated effector or memory cells generally requires both TCR engagement of antigen as described above as well as engagement of costimulatory proteins. Optimal induction of CTL responses usually requires “help” in the form of cytokines from CD4⁺ T lymphocytes which recognize antigen associated with MHC class II molecules via TCR and CD4 engagement.

Larder, et al., (1987, Nature 327: 716-717) and Larder, et al., (1989, Proc. Natl. Acad. Sci. 86: 4803-4807) disclose site specific mutagenesis of HIV-1 RT and the effect such changes have on in vitro activity and infectivity related to interaction with known inhibitors of RT.

Davies, et al. (1991, Science 252:, 88-95) disclose the crystal structure of the RNase H domain of HIV-1 Pol.

Schatz, et al. (1989, FEBS Lett. 257: 311-314) disclose that mutations Glu478Gln and His539Phe in a complete HIV-1 RT/RNase H DNA fragment results in defective RNase activity without effecting RT activity.

Mizrahi, et al. (1990, Nucl. Acids. Res. 18: pp. 5359-5353) disclose additional mutations Asp443Asn and Asp498Asn in the RNase region of the pol gene which also results in defective RNase activity. The authors note that the Asp498Asn mutant was difficult to characterize due to instability of this mutant protein.

Leavitt, et al. (1993, J. Biol. Chem. 268: 2113-2119) disclose several mutations, including a Asp64Val mutation, which show differing effect on HIV-1 integrase (IN) activity.

Wiskerchen, et al. (1995, J. Virol. 69: 376-386) disclose singe and double mutants, including mutation of aspartic acid residues which effect HIV-1 IN and viral replication functions.

It would be of great import in the battle against AIDS to produce a prophylactic- and/or therapeutic-based HIV vaccine which generates a strong cellular immune response against an HIV infection. The present invention addresses and meets this needs by disclosing a class of DNA vaccines based on host delivery and expression of modified versions of the HIV-1 gene, pol.

### SUMMARY OF THE INVENTION

The present invention relates to synthetic DNA molecules (also referred to herein as “polynucleotides”) and associated DNA vaccines (also referred to herein as “polynucleotide vaccines”) which elicit cellular immune and humoral responses upon administration to the host, including primates and especially humans, and also including a non-human mammal of commercial or domestic veterinary importance. An effect of the cellular immune-directed vaccines of the present invention should be the lower transmission rate to previously uninfected individuals and/or reduction in the levels of the viral loads within an infected individual, so as to prolong the asymptomatic phase of HIV-1 infection. In particular, the present invention relates to DNA vaccines which encode various forms of HIV-1 Pol, wherein administration, intracellular delivery and expression of the HIV-1 Pol gene of interest elicits a host CTL and Th response. The preferred synthetic DNA molecules of the present invention encode codon optimized versions of wild type HIV-1 Pol, codon optimized versions of HIV-1 Pol fusion proteins, and codon optimized versions of HIV-1 Pol proteins and fusion protein, including but not limited to pol modifications involving residues within the catalytic regions responsible for RT, RNase and IN activity within the host cell.

A particular embodiment of the present invention relates to codon optimized wt-pol DNA constructs wherein DNA sequences encoding the protease (PR) activity are deleted, leaving codon optimized “wild type” sequences which encode RT (reverse transcriptase and RNase H activity) and IN integrase activity. The nucleotide sequence of a DNA molecule which encodes this protein is disclosed herein as SEQ ID NO: 1 and the corresponding amino acid sequence of the expressed protein is disclosed herein as SEQ ID NO: 2.

The present invention preferably relates to a HIV-1 DNA pol construct which is devoid of DNA sequences encoding any PR activity, as well as containing a mutation(s) which at least partially, and preferably substantially, abolishes RT, RNase and/or IN activity. One type of HIV-1 pol mutant may include but is not limited to a mutated DNA molecule comprising at least one nucleotide substitution which results in a point mutation which effectively alters an active site within the RT, RNase and/or IN regions of the expressed protein, resulting in at least substantially decreased enzymatic activity for the RT, RNase H and/or IN functions of HIV-1 Pol. In a preferred embodiment of this portion of the invention, a HIV-1 DNA pol construct contains a mutation or mutations within the Pol coding region which effectively abolishes RT, RNase H and IN activity. An especially preferable HIV-1 DNA pol construct in a DNA molecule which contains at least one point mutation which alters the active site of the RT, RNase H and IN domains of Pol, such that each activity is at least substantially abolished. Such a HIV-1 Pol mutant will most likely comprise at least one point mutation in or around each catalytic domain responsible for RT, RNase H and IN activity, respectfully. To this end, an especially preferred HIV-1 DNA pol construct is exemplified herein and contains nine codon substitution mutations which results in an inactivated Pol protein (IA Pol: SEQ ID NO: 4, FIGS. 2A-C) which has no PR, RT, RNase or IN activity, wherein three such point mutations reside within each of the RT, RNase and IN catalytic domains. Any combination of the mutations disclosed herein may suitable and therefore may be utilized as an IA-Pol-based vaccine of the present invention. While addition and deletion mutations are contemplated and within the scope of the invention, the preferred mutation is a point mutation resulting in a substitution of the wild type amino acid with an alternative amino acid residue.

Another aspect of the present invention is to generate HIV-1 Pol-based vaccine constructions which comprise a eukaryotic trafficking signal peptide such as the leader peptide from human tPA. To this end, the present invention relates to a DNA molecule which encodes a codon optimized wt-pol DNA construct wherein the protease (PR) activity is deleted and a human tPA leader sequence is fused to the 5′ end of the coding region. A DNA molecule which encodes this protein is disclosed herein as SEQ ID NO: 5, the open reading frame disclosed herein as SEQ ID NO: 6.

The present invention especially relates to a HIV-1 Pol mutant such as IA-Pol (SEQ ID NO: 4) which comprises a leader peptide, such as the human tpA leader, at the amino terminal portion of the protein, which may effect cellular trafficking and hence, immunogenicity of the expressed protein within the host cell. Any such HIV-1 DNA pol mutant disclosed in the above paragraphs is suitable for fusion downstream of a leader peptide, including but by no means limited to the human tPA leader sequence. Therefore, any such leader peptide-based HIV-1 pol mutant construct may include but is not limited to a mutated DNA molecule which effectively alters the catalytic activity of the RT, RNase and/or IN region of the expressed protein, resulting in at least substantially decreased enzymatic activity one or more of the RT, RNase H and/or IN functions of HIV-1 Pol. In a preferred embodiment of this portion of the invention, a leader peptide/HIV-1 DNA pol construct contains a mutation or mutations within the Pol coding region which effectively abolishes RT, RNase H and IN activity. An especially preferable HIV-1 DNA pol construct is a DNA molecule which contains at least one point mutation which alters the active site and catalytic activity within the RT, RNase H and IN domains of Pol, such that each activity is at least substantially abolished, and preferably totally abolished. Such a HIV-1 Pol mutant will most likely comprise at least one point mutation in or around each catalytic domain responsible for RT, RNase H and IN activity, respectfully. An especially preferred embodiment of this portion of the invention relates to a human tPA leader fused to the IA-Pol protein comprising the nine mutations shown in Table 1. The DNA molecule is disclosed herein as SEQ ID NO: 7 and the expressed tPA-IA Pol protein comprises a fusion junction as shown in FIG. 3. The complete amino acid sequence of the expressed protein is set forth in SEQ ID NO: 8.

The present invention also relates to a substantially purified protein expressed from the DNA polynucleotide vaccines of the present invention, especially the purified proteins set forth below as SEQ ID NOs: 2, 4, 6, and 8. These purified proteins may be useful as protein-based HIV vaccines.

The present invention also relates to non-codon optimized versions of DNA molecules and associated polynucleotides and associated DNA vaccines which encode the various wild type and modified forms of the HIV Pol protein disclosed herein. Partial or fully codon optimized DNA vaccine expression vector constructs are preferred, but it is within the scope of the present invention to utilize “non-codon optimized” versions of the constructs disclosed herein, especially modified versions of HIV Pol which are shown to promote a substantial cellular immune and humoral immune responses subsequent to host administration.

The DNA backbone of the DNA vaccines of the present invention are preferably DNA plasmid expression vectors. DNA plasmid expression vectors utilized in the present invention include but are not limited to constructs which comprise the cytomegalovirus promoter with the intron A sequence (CMV-intA) and a bovine growth hormone transcription termination sequence. In addition, DNA plasmid vectors of the present invention preferably comprise an antibiotic resistance marker, including but not limited to an ampicillin resistance gene, a neomycin resistance gene or any other pharmaceutically acceptable antibiotic resistance marker. In addition, an appropriate polylinker cloning site and a prokaryotic origin of replication sequence are also preferred. Specific DNA vectors exemplified herein include V1, V1J (SEQ ID NO: 13), V1Jneo (SEQ ID NO: 14), V1Jns (FIG. 1A, SEQ ID NO: 15), V1R (SEQ ID NO: 26), and any of the aforementioned vectors wherein a nucleotide sequence encoding a leader peptide, preferably the human tpA leader, is fused directly downstream of the CMV-intA promoter, including but not limited to V1Jns-tpa, as shown in FIG. 1B and SEQ ID NO: 28.

The present invention especially relates to a DNA vaccine and a pharmaceutically active vaccine composition which contains this DNA vaccine, and the use as prophylactic and/or therapeutic vaccine for host immunization, preferably human host immunization, against an HIV infection or to combat an existing HIV condition. These DNA vaccines are represented by codon optimized DNA molecules encoding codon optimized HIV-1 Pol (e.g. SEQ ID NO: 2), codon optimized HIV-1 Pol fused to an amino terminal localized leader sequence (e.g. SEQ ID NO: 6), and especially preferable, and the essence of the present invention, biologically inactive Pol proteins (IA Pol; e.g., SEQ ID NO: 4) devoid of significant PR, RT, RNase or IN activity associated with wild type Pol and a concomitant construct which contains a leader peptide at the amino terminal region of the IA Pol protein. These constructs are ligated within an appropriate DNA plasmid vector, with or without a nucleotide sequence encoding a functional leader peptide. Preferred DNA vaccines of the present invention comprise codon optimized DNA molecules encoding codon optimized HIV-1 Pol and inactivated version of Pol, ligated in DNA vectors disclosed herein, or any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO: 28.

Therefore, the present invention relates to DNA vaccines which include, but are in no way limited to V1Jns-WTPol (comprising the DNA molecule encoding WT Pol, as set forth in SEQ ID NO: 2), V1Jns-tPA-WTPol, (comprising the DNA molecule encoding tPA Pol, as set forth in SEQ ID NO: 6), V1Jns-IAPol (comprising the DNA molecule encoding IA Pol, as set forth in SEQ ID NO: 4), and V1Jns-tPA-IAPol, (comprising the DNA molecule encoding tPA-IA Pol, as set forth in SEQ ID NO: 8). Especially preferred are V1Jns-IAPol and V1Jns-tPA-IAPol, as exemplified in Example Section 2.

The present invention also relates to HIV Pol polynucleotide pharmaceutical products, as well as the production and use thereof, wherein the DNA vaccines are formulated with an adjuvant or adjuvants which may increase immunogenicity of the DNA polynucleotide vaccines of the present invention, namely by promoting an enhanced cellular and/or humoral response subsequent to inoculation. A preferred adjuvant is an aluminum phosphate-based adjuvant or a calcium phosphate based adjuvant, with an aluminum phosphate adjuvant being especially preferred. Another preferred adjuvant is a non-ionic block copolymer, preferably comprising the blocks of polyoxyethylene (POE) and polyoxypropylene (POP) such as a POE-POP-POE block copolymer. These adjuvanted forms comprising the DNA vaccines disclosed herein are useful in increasing cellular responses to DNA vaccination.

As used herein, a DNA vaccine or DNA polynucleotide vaccine is a DNA molecule (i.e., “nucleic acid”, “polynucleotide”) which contains essential regulatory elements such that upon introduction into a living, vertebrate cell, it is able to direct the cellular machinery to produce translation products encoded by the respective pol genes of the present invention.

### BRIEF DESCRIPTION OF THE FIGURES

FIGS. 1A-B shows schematic representation of DNA vaccine expression vectors V1Jns (A) and V1Jns-tPA (B) utilized for HIV-1 pol and HIV-1 modified pol constructs.

FIGS. 2A-C shows the nucleotide (SEQ ID NO: 3) and amino acid sequence (SEQ ID NO: 4) of IA-Pol. Underlined codons and amino acids denote mutations, as listed in Table 1.

FIG. 3 shows the codon optimized nucleotide and amino acid sequences through the fusion junction of tPA-IA-Pol (contained within SEQ ID NOs: 7 and 8, respectively). The underlined portion represents the NH₂-terminal region of IA-Pol.

FIG. 4 shows generation of a humoral response (measured as the geometric means of anti-RT endpoint titers) from mice immunized with one or two doses of codon optimized V1Jns-IApol and V1Jns-tpa-IApol. A portion of mice that received 30 ug of each plasmid was boosted at T=8 wks; sera from all mice were collected at 4 wk post dose 2.

FIG. 5 shows the number of IFN-gamma secreting cells per 10e6 cells following stimulation with pools of either CD4⁺ (aa641-660, aa731-750) or CD8⁺ (aa201-220, aa311-330, aa571-590, aa781-800) specific peptides of splenocytes (pool of 5 spleens/cohort) from control mice and those vaccinated with increasing single dose of codon optimized V1Jns-IApol or 30 ug of codon optimized V1Jns-tpa-IApol (13 wks post dose 1). Mice (n=5) vaccinated with a second dose of 30 ug of either plasmid were analyzed in an Elispot assay at 6 wks post dose 2. Reported are the sums of the number of spots stimulated by each individual CD8⁺ peptides because the spots in the wells to which the pool was added are too dense to acquire accurate counts. The CD4⁺ cell counts are taken from the responses to the peptide pool. Error bars represent standard deviations for counts from triplicate wells per sample per antigen.

FIGS. 6A-C shows ELIspot analysis of peripheral blood cells collected from rhesus macaques immunized three times (T=0, 4, 8 wks) with 5 mgs of codon optimized HIV-1 Pol expressing plasmids. Antigen-specific IFN-gamma secretion was stimulated by adding one of two pools consisting of 20-mer peptides derived from vaccine sequence (mpol-1, aa1-420; mpol-2, aa411-850). (A) Frequencies of spot-forming cells (SFC) as a function of time for 3 monkeys (Tag No. 94R008, 94R013, 94R033) vaccinated with V1Jns-IApol. The reported values are corrected for background responses without peptide restimulation. (B) Frequencies of spot-forming cells (SFC) as a function of time for 3 monkeys (Tag No. 920078, 920073, 94R028) vaccinated with 5 mgs of V1Jns-tpa-IApol. (C) ELIspot responses were also measured from a monkey (920072) that did not receive any immunization.

FIGS. 7A-B show bulk CTL killing from rhesus macaques immunized with codon optimized V1Jns-IApol (A)or codon optimized V1Jns-tpa-IApol (B) at 8 weeks following the third vaccination. Restimulation was performed using recombinant vaccinia virus expressing pol and target cells were prepared by pulsing with the peptide pools, mpol-1 and mpol-2.

FIG. 8 shows detection of in vitro pol expression from cell lysates of 293 cells transfected with 10 ug of various pol constructs. Bands were detected using anti-serum from an HIV-1 seropositive human subject. Equal amounts of total protein were loaded for each lane. The lanes contain the lysates from cells transfected with the following: 1: mock; 2: V1Jns-wt-pol; 3: V1Jns-IApol (codon optimized); 4: V1Jns-tpa-IApol (codon optimized); 5: V1Jns-tpa-pol (codon optimized); 6: V1R-wt-pol (codon optimized); 7: blank; and 8: 80 ng RT.

FIG. 9 shows the geometric mean anti-RT titers (GMT) plus the standard errors of the geometric means for cohorts of 5 mice that received one (open circles) or two doses (solid circles) of 1, 10, 100 μg of V1R-wt-pol (codon optimized) or V1Jns-wt-pol. Sera from all animals were collected at 2 weeks post dose 2 (or 7 wks post dose 1) and assayed simultaneously. Statistical analyses were performed to compare cohorts that received the same amount and number of immunization of either plasmids; p values (two-tail) less than 5% are above the bars the connect the correlated cohorts to reflect statistically significant differences.

FIG. 10 shows cellular immune responses in BALB/c mice vaccinated i.m. with 1 (pd1) or 2 (pd2) doses of varying amounts of either wt-pol (virus derived) or wt-pol (codon optimized) plasmids. At 3 wks post dose 2, frequencies of IFN-γ-secreting splenocytes are determined from pools of 5 spleens per cohort against mixtures of either CD4⁺ peptides (aa21-40, aa411-430, aa531-550, aa641-660, aa731-750, aa771-790) or CD8⁺ peptides (aa201-220, aa311-330) at 4 μg/mL final concentration per peptide.

### DETAILED DESCRIPTION OF THE INVENTION

The present invention relates to synthetic DNA molecules and associated DNA vaccines which elicit CTL and Th cellular immune responses upon administration to the host, including primates and especially humans. An effect of the cellular immune-directed vaccines of the present invention should be a lower transmission rate to previously uninfected individuals and/or reduction in the levels of the viral loads within an infected individual, so as to prolong the asymptomatic phase of HIV-1 infection. In particular, the present invention relates to DNA vaccines which encode various forms of HIV-1 Pol, wherein administration, intracellular delivery and expression of the HIV-1 Pol gene of interest elicits a host CTL and Th response. The preferred synthetic DNA molecules of the present invention encode codon optimized wild type Pol (without Pro activity) and various codon optimized inactivated HIV-1 Pol proteins. The HIV-1 pol constructs disclosed herein are especially preferred for pharmaceutical uses, especially for human administration as a DNA vaccine. The HIV-1 genome employs predominantly uncommon codons compared to highly expressed human genes. Therefore, the pol open reading frame has been synthetically manipulated using optimal codons for human expression. As noted above, a preferred embodiment of the present invention relates to DNA molecules which comprise a HIV-1 pol open reading frame, whether encoding full length pol or a modification or fusion as described herein, wherein the codon usage has been optimized for expression in a mammal, especially a human.

The synthetic pol gene disclosed herein comprises the coding sequences for the reverse transcriptase (or RT which consists of a polymerase and RNase H activity) and integrase (IN). The protein sequence is based on that of H×b2r, a clonal isolate of IIIB; this sequence has been shown to be closest to the consensus clade B sequence with only 16 nonidentical residues out of 848 (Korber, et al., 1998, Human retroviruses and AIDS, Los Alamos National Laboratory, Los Alamos, N. Mex.). The skilled artisan will understand after review of this specification that any available HIV-1 or HIV-2 strain provides a potential template for the generation of HIV pol DNA vaccine constructs disclosed herein. It is further noted that the protease gene is excluded from the DNA vaccine constructs of the present invention to insure safety from any residual protease activity in spite of mutational inactivation. The design of the gene sequences for both wild-type (wt-pol) and inactivated pol (IA-pol) incorporates the use of human preferred (“humanized”) codons for each amino acid residue in the sequence in order to maximize in vivo mammalian expression (Lathe, 1985, J. Mol. Biol. 183:1-12). As can be discerned by inspecting the codon usage in SEQ ID NOs: 1, 3, 5 and 7, the following codon usage for mammalian optimization is preferred: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (ITC) and Tyr (TAC). For an additional discussion relating to mammalian (human) codon optimization, see WO 97/31115 (PCT/US97/02294), which is hereby incorporated by reference. It is intended that the skilled artisan may use alternative versions of codon optimization or may omit this step when generating HIV pol vaccine constructs within the scope of the present invention. Therefore, the present invention also relates to non-codon optimized versions of DNA molecules and associated DNA vaccines which encode the various wild type and modified forms of the HIV Pol protein disclosed herein. However, codon optimization of these constructs is a preferred embodiment of this invention.

A particular embodiment of the present invention relates to codon optimized wt-pol DNA constructs (herein, “wt-pol” or “wt-pol (codon optimized))” wherein DNA sequences encoding the protease (PR) activity are deleted, leaving codon optimized “wild type” sequences which encode RT (reverse transcriptase and RNase H activity) and IN integrase activity. A DNA molecule which encodes this protein is disclosed herein as SEQ ID NO: 1, the open reading frame being contained from an initiating Met residue at nucleotides 10-12 to a termination codon from nucleotides 2560-2562. SEQ ID NO: 1 is as follows:

| | |
|---|---|
| AGATCTACCA TGGCCCCCAT CTCCCCCATT GAGACTGTGC CTGTGAAGCT GAAGCCTGGC | (SEQ ID NO:1) |
| ATGGATGGCC CCAAGGTGAA GCAGTGGCCC CTGACTGAGG AGAAGATCAA GGCCCTGGTG | |
| GAAATCTGCA CTGAGATGGA GAAGGAGGGC AAAATCTCCA AGATTGGCCC CGAGAACCCC | |
| TACAACACCC CTGTGTTTGC CATCAAGAAG AAGGACTCCA CCAAGTGGAG GAAGCTGGTG | |
| GACTTCAGGG AGCTGAACAA GAGGACCCAG GACTTCTGGG AGGTGCAGCT GGGCATCCCC | |
| CACCCCGCTG GCCTGAAGAA GAAGAAGTCT GTGACTGTGC TGGATGTGGG GGATGCCTAC | |
| TTCTCTGTGC CCCTGGATGA GGACTTCAGG AAGTACACTG CCTTCACCAT CCCCTCCATC | |
| AACAATGAGA CCCCTGGCAT CAGGTACCAG TACAATGTGC TGCCCCAGGG CTGGAAGGGC | |
| TCCCCTGCCA TCTTCCAGTC CTCCATGACC AAGATCCTGG AGCCCTTCAG GAAGCAGAAC | |
| CCTGACATTG TGATCTACCA GTACATGGAT GACCTGTATG TGGGCTCTGA CCTGGAGATT | |
| GGGCAGCACA GGACCAAGAT TGAGGAGCTG AGGCAGCACC TGCTGAGGTG GGGCCTGACC | |
| ACCCCTGACA AGAAGCACCA GAAGGAGCCC CCCTTCCTGT GGATGGGCTA TGAGCTGCAC | |
| CCCGACAAGT GGACTGTGCA GCCCATTGTG CTGCCTGAGA AGGACTCCTG GACTGTGAAT | |
| GACATCCAGA AGCTGGTGGG CAAGCTGAAC TGGGCCTCCC AAATCTACCC TGGCATCAAG | |
| GTGAGGCAGC TGTGCAAGCT GCTGAGGGGC ACCAAGGCCC TGACTGAGGT GATCCCCCTG | |
| ACTGAGGAGG CTGAGCTGGA GCTGGCTGAG AACAGGGAGA TCCTGAAGGA GCCTGTGCAT | |
| GGGGTGTACT ATGACCCCTC CAAGGACCTG ATTGCTGAGA TCCAGAAGCA GGGCCAGGGC | |
| CAGTGGACCT ACCAAATCTA CCAGGAGCCC TTCAAGAACC TGAAGACTGG CAAGTATGCC | |
| AGGATGAGGG GGGCCCACAC CAATGATGTG AAGCAGCTGA CTGAGGCTGT GCAGAAGATC | |
| ACCACTGAGT CCATTGTGAT CTGGGGCAAG ACCCCCAAGT TCAAGCTGCC CATCCAGAAG | |
| GAGACCTGGG AGACCTGGTG GACTGAGTAC TGGCAGGCCA CCTGGATCCC TGAGTGGGAG | |
| TTTGTGAACA CCCCCCCCCT GGTGAAGCTG TGGTACCAGC TGGAGAAGGA GCCCATTGTG | |
| GGGGCTGAGA CCTTCTATGT GGATGGGGCT GCCAACAGGG AGACCAAGCT GGGCAAGGCT | |
| GGCTATGTGA CCAACAGGGG CAGGCAGAAG GTGGTGACCC TGACTGACAC CACCAACCAG | |
| AAGACTGAGC TCCAGGCCAT CTACCTGGCC CTCCAGGACT CTGGCCTGGA GGTGAACATT | |
| GTGACTGACT CCCAGTATGC CCTGGGCATC ATCCAGGCCC AGCCTGATCA GTCTGAGTCT | |
| GAGCTGGTGA ACCAGATCAT TGAGCAGCTG ATCAAGAAGG AGAAGGTGTA CCTGGCCTGG | |
| GTGCCTGCCC ACAAGGGCAT TGGGGGCAAT GAGCAGGTGG ACAAGCTGGT GTCTGCTGGC | |
| ATCAGGAAGG TGCTGTTCCT GGATGGCATT GACAAGGCCC AGGATGAGCA TGAGAAGTAC | |
| CACTCCAACT GGAGGGCTAT GGCCTCTGAC TTCAACCTGC CCCCTGTGGT GGCTAAGGAG | |
| ATTGTGGCCT CCTGTGACAA GTGCCAGCTG AAGGGGGAGG CCATGCATGG GCAGGTGGAC | |
| TGCTCCCCTG GCATCTGGCA GCTGGACTGC ACCCACCTGG AGGGCAAGGT GATCCTGGTG | |
| GCTGTGCATG TGGCCTCCGG CTACATTGAG GCTGAGGTGA TCCCTGCTGA GACACGCCAG | |
| GAGACTGCCT ACTTCCTGCT GAAGCTGGCT GGCAGGTGGC CTGTGAAGAC CATCCACACT | |
| GACAATGGCT CCAACTTCAC TGGGGCCACA GTGAGGGCTG CCTGCTGGTG GGCTGGCATC | |
| AAGCAGGAGT TTGGCATCCC CTACAACCCC CAGTCCCAGG GGGTGGTGGA GTCCATGAAC | |
| AAGGAGCTGA AGAAGATCAT TGGGCAGGTG AGGGACCAGG CTGAGCACCT GAAGACAGCT | |
| GTGCAGATGG CTGTGTTCAT CCACAACTTC AAGAGGAAGG GGGGCATCGG GGGCTACTCC | |
| GCTGGGGAGA GGATTGTGGA CATCATTGCC ACAGACATCC AGACCAAGGA GCTCCAGAAG | |
| CAGATCACCA AGATCCAGAA CTTCAGGGTG TACTACAGGG ACTCCAGGAA CCCCCTGTGG | |
| AAGGGCCCTG CCAAGCTGCT GTGGAAGGGG GAGGGGGCTG TGGTGATCCA GGACAACTCT | |
| GACATCAAGG TGGTGCCCAG GAGGAAGGCC AAGATCATCA GGGACTATGG CAAGCAGATG | |
| GCTGGGGATG ACTGTGTGGC CTCCAGGCAG GATGAGGACT AAAGCCCGGG CAGATCT. | |

The open reading frame of the wild type pol construct disclosed as SEQ ID NO: 1 contains 850 amino acids, disclosed herein as SEQ ID NO: 2, as follows:

| | |
|---|---|
| Met Ala Pro Ile Ser Pro Ile Glu Thr Val Pro Val Lys Leu Lys Pro | (SEQ ID NO:2) |
| Gly Met Asp Gly Pro Lys Val Lys Gln Trp Pro Leu Thr Glu Glu Lys | |
| Ile Lys Ala Leu Val Glu Ile Cys Thr Glu Met Glu Lys Glu Gly Lys | |
| Ile Ser Lys Ile Giy Pro Glu Asn Pro Tyr Asn Thr Pro Val Phe Ala | |
| Ile Lys Lys Lys Asp Ser Thr Lys Trp Arg Lys Leu Val Asp Phe Arg | |
| Glu Leu Asn Lys Arg Thr Gln Asp Phe Trp Glu Val Gln Leu Gly Ile | |
| Pro His Pro Ala Gly Leu Lys Lys Lys Lys Ser Val Thr Val Leu Asp | |
| Val Gly Asp Ala Tyr Phe Ser Val Pro Leu Asp Glu Asp Phe Arg Lys | |
| Tyr Thr Ala Phe Thr Ile Pro Ser Ile Asn Asn Glu Thr Pro Gly Ile | |
| Arg Tyr Gln Tyr Asn Val Leu Pro Gln Gly Trp Lys Gly Ser Pro Ala | |
| Ile Phe Gln Ser Ser Met Thr Lys Ile Leu Glu Pro Phe Arg Lys Gln | |
| Asn Pro Asp Ile Val Ile Tyr Gln Tyr Met Asp Asp Leu Tyr Val Gly | |
| Ser Asp Leu Glu Ile Gly Gln His Arg Thx Lys Ile Glu Glu Leu Arg | |
| Gln His Leu Leu Arg Trp Gly Leu Tbr Thr Pro Asp Lys Lys His Gln | |
| Lys Glu Pro Pro Phe Leu Trp Met Gly Tyr Glu Leu His Pro Asp Lys | |
| Trp Thr Val Gln Pro Ile Val Leu Pro Glu Lys Asp Ser Trp Thr Val | |
| Asn Asp Ile Gln Lys Leu Val Gly Lys Leu Asn Trp Ala Ser Gln Ile | |
| Tyr Pro Gly Ile Lys Val Arg Gln Leu Cys Lys Leu Leu Arg Gly Thr | |
| Lys Ala Leu Thr Glu Val Ile Pro Leu Thr Glu Glu Ala Glu Leu Glu | |
| Leu Ala Glu Asn Arg Glu Ile Leu Lys Glu Pro Val His Gly Val Tyr | |
| Tyr Asp Pro Ser Lys Asp Leu Ile Ala Glu Ile Gln Lys Gln Gly Gln | |
| Gly Gln Trp Thr Tyr Gln Ile Tyr Gln Glu Pro Phe Lys Asn Leu Lys | |
| Thr Gly Lys Tyr Ala Arg Met Arg Gly Ala His Thr Asn Asp Val Lys | |
| Gln Leu Thr Glu Ala Val Gln Lys Ile Thr Thr Glu Ser Ile Val Ile | |
| Trp Gly Lys Thr Pro Lys Phe Lys Leu Pro Ile Gln Lys Glu Thr Trp | |
| Glu Thr Trp Trp Thr Glu Tyr Trp Gln Ala Thr Trp Ile Pro Glu Trp | |
| Glu Phe Val Asn Thr Pro Pro Leu Val Lys Leu Trp Tyr Gln Leu Glu | |
| Lys Glu Pro Ile Val Gly Ala Glu Thr Phe Tyr Val Asp Gly Ala Ala | |
| Asn Arg Glu Thr Lys Leu Gly Lys Ala Gly Tyr Val Thr Asn Arg Gly | |
| Arg Gln Lys Val Val Thr Leu Thr Asp Thr Thr Asn Gln Lys Thr Glu | |
| Leu Gln Ala Ile Tyr Leu Ala Leu Gln Asp Ser Gly Leu Glu Val Asn | |
| Ile Val Thr Asp Ser Gln Tyr Ala Leu Gly Ile Ile Gln Ala Gln Pro | |
| Asp Gln Ser Glu Ser Glu Leu Val Asn Gln Ile Ile Glu Gln Leu Ile | |
| Lys Lys Glu Lys Val Tyr Leu Ala Trp Val Pro Ala His Lys Gly Ile | |
| Gly Gly Asn Glu Gln Val Asp Lys Leu Val Ser Ala Gly Ile Arg Lys | |
| Val Leu Phe Leu Asp Gly Ile Asp Lys Ala Gln Asp Glu His Glu Lys | |
| Tyr His Ser Asn Trp Arg Ala Met Ala Ser Asp Phe Asn Leu Pro Pro | |
| Val Val Ala Lys Glu Ile Val Ala Ser Cys Asp Lys Cys Gln Leu Lys | |
| Gly Glu Ala Met His Gly Gln Val Asp Cys Ser Pro Gly Ile Trp Gln | |
| Leu Asp Cys Thr His Leu Glu Gly Lys Val Ile Leu Val Ala Val His | |
| Val Ala Ser Gly Tyr Ile Glu Ala Glu Val Ile Pro Ala Glu Thr Gly | |
| Gln Glu Thr Ala Tyr Phe Leu Leu Lys Leu Ala Gly Arg Trp Pro Val | |
| Lys Thr Ile His Thr Asp Asn Gly Ser Asn Phe Thr Gly Ala Thr Val | |
| Arg Ala Ala Cys Trp Trp Ala Gly Ile Lys Gln Glu Phe Gly Ile Pro | |
| Tyr Asn Pro Gln Ser Gln Gly Val Val Glu Ser Met Asn Lys Glu Leu | |
| Lys Lys Ile Ile Gly Gln Val Arg Asp Gln Ala Glu His Leu Lys Thr | |
| Ala Val Gln Met Ala Val Phe Ile His Asn Phe Lys Arg Lys Gly Gly | |
| Ile Gly Gly Tyr Ser Ala Gly Glu Arg Ile Val Asp Ile Ile Ala Thr | |
| Asp Ile Gln Thr Lys Glu Leu Gln Lys Gln Ile Thr Lys Ile Gln Asn | |
| Phe Arg Val Tyr Tyr Arg Asp Ser Arg Asn Pro Leu Trp Lys Gly Pro | |
| Ala Lys Leu Leu Trp Lys Gly Glu Gly Ala Val Val Ile Gln Asp Asn | |
| Ser Asp Ile Lys Val Val Pro Arg Arg Lys Ala Lys Ile Ile Arg Asp | |
| Tyr Gly Lys Gln Met Ala Gly Asp Asp Cys Val Ala Ser Arg Gln Asp | |
| Glu Asp. | |

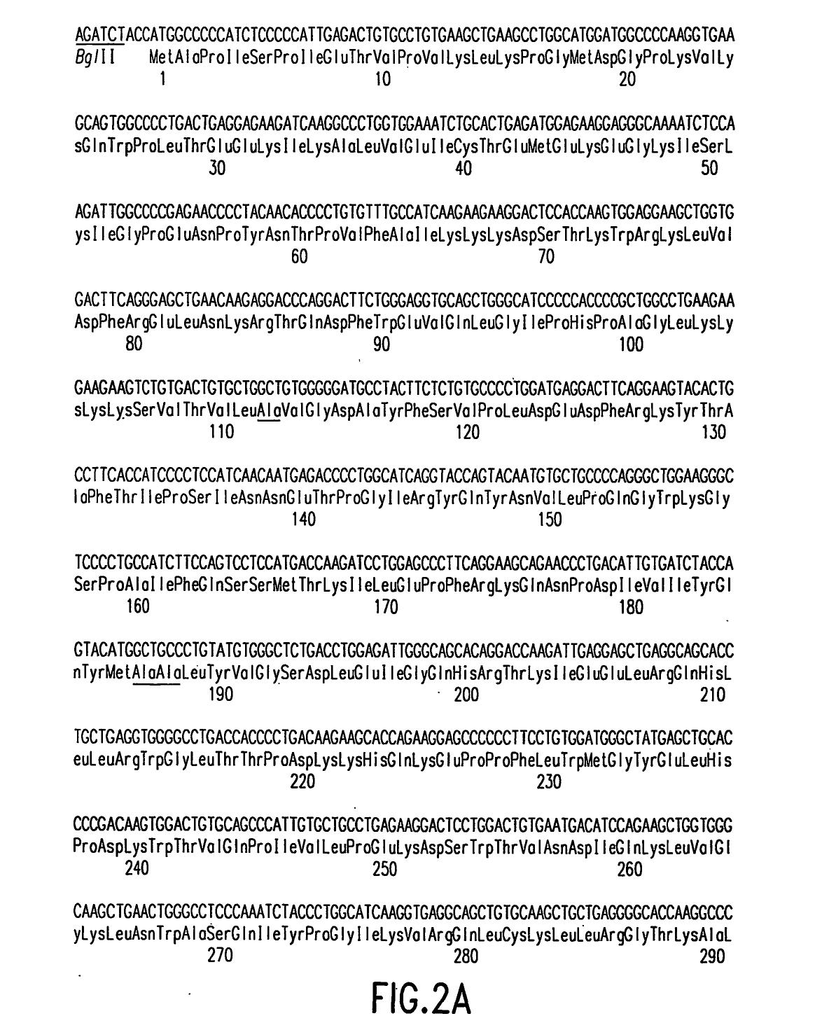

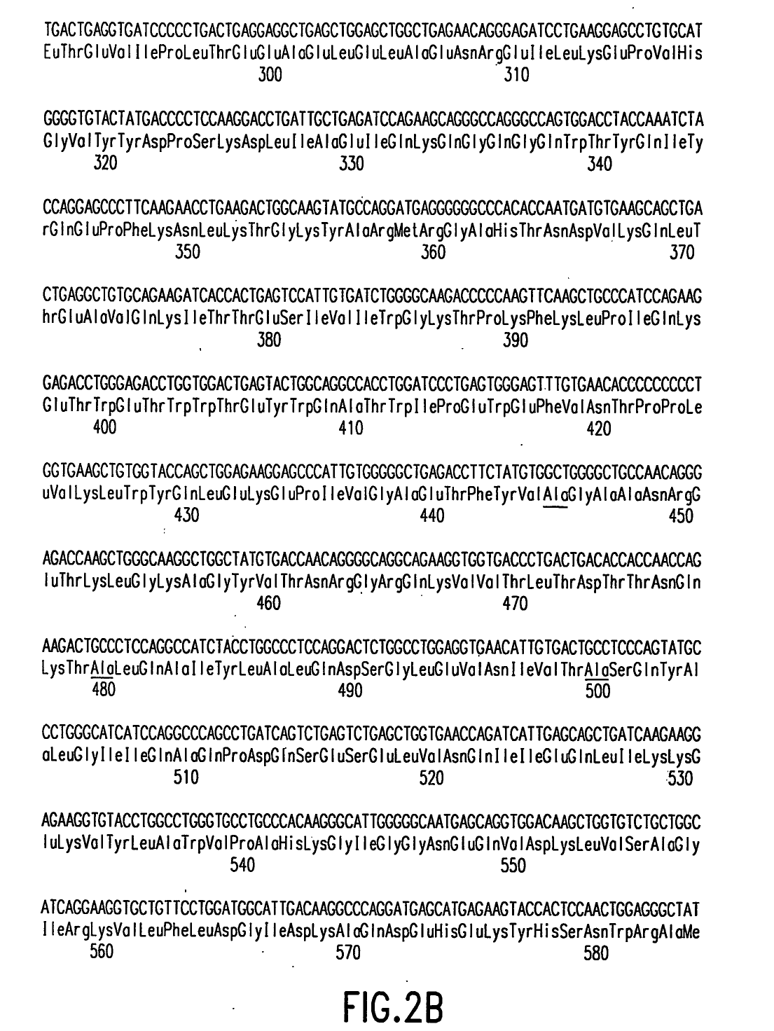

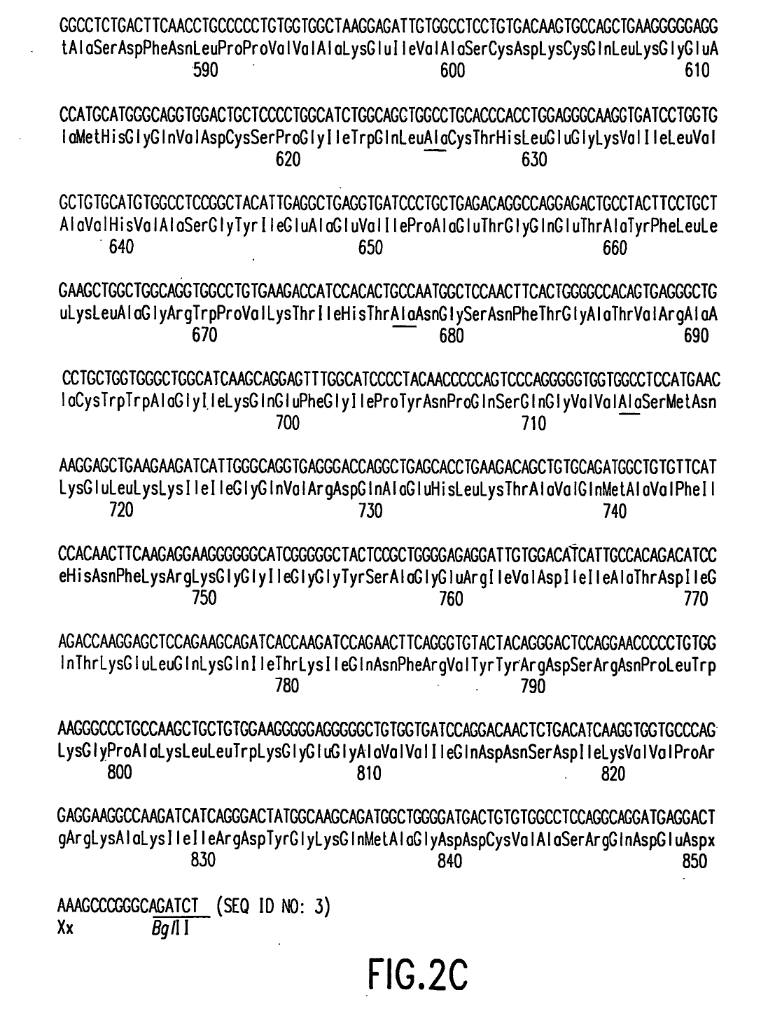

The present invention especially relates to a codon optimized HIV-1 DNA pol construct wherein, in addition to deletion of the portion of the wild type sequence encoding the protease activity, a combination of active site residue mutations are introduced which are deleterious to HIV-1 pol (RT-RH-IN) activity of the expressed protein. Therefore, the present invention preferably relates to a HIV-1 DNA pol construct which is devoid of DNA sequences encoding any PR activity, as well as containing a mutation(s) which at least partially, and preferably substantially, abolishes RT, RNase and/or IN activity. One type of HIV-1 pol mutant may include but is not limited to a mutated DNA molecule comprising at least one nucleotide substitution which results in a point mutation which effectively alters an active site within the RT, RNase and/or IN regions of the expressed protein, resulting in at least substantially decreased enzymatic activity for the RT, RNase H and/or IN functions of HIV-1 Pol. In a preferred embodiment of this portion of the invention, a HIV-1 DNA pol construct contains a mutation or mutations within the Pol coding region which effectively abolishes RT, RNase H and IN activity. An especially preferable HIV-1 DNA pol construct in a DNA molecule which contains at least one point mutation which alters the active site of the RT, RNase H and IN domains of Pol, such that each activity is at least substantially abolished. Such a HIV-1 Pol mutant will most likely comprise at least one point mutation in or around each catalytic domain responsible for RT, RNase H and IN activity, respectfully. To this end, an especially preferred HIV-1 DNA pol construct is exemplified herein and contains nine codon substitution mutations which results in an inactivated Pol protein (IA Pol: SEQ ID NO: 4, FIGS. 2A-C) which has no PR, RT, RNase or IN activity, wherein three such point mutations reside within each of the RT, RNase and IN catalytic domains. Therefore, an especially preferred exemplification is a DNA molecule which encodes IA-pol, which contains all nine mutations as shown below in Table 1. An additional preferred amino acid residue for substitution is Asp551, localized within the RNase domain of Pol. Any combination of the mutations disclosed herein may suitable and therefore may be utilized as an IA-Pol-based vaccine of the present invention. While addition and deletion mutations are contemplated and within the scope of the invention, the preferred mutation is a point mutation resulting in a substitution of the wild type amino acid with an alternative amino acid residue.

TABLE 1

| wt aa | aa residue | mutant aa | enzyme function |
|---|---|---|---|
| Asp | 112 | Ala | RT |
| Asp | 187 | Ala | RT |
| Asp | 188 | Ala | RT |
| Asp | 445 | Ala | RNase H |
| Glu | 480 | Ala | RNase H |
| Asp | 500 | Ala | RNase H |
| Asp | 626 | Ala | IN |
| Asp | 678 | Ala | IN |
| Glu | 714 | Ala | IN |

It is preferred that point mutations be incorporated into the IApol mutant vaccines of the present invention so as to lessen the possibility of altering epitopes in and around the active site(s) of HIV-1 Pol.

To this end, SEQ ID NO: 3 discloses the nucleotide sequence which codes for a codon optimized pol in addition to the nine mutations shown in Table 1, disclosed as follows, and referred to herein as “IApol” :

| | |
|---|---|
| AGATCTACCA TGGCCCCCAT CTCCCCCATT GAGACTGTGC CTGTGAAGCT GAAGCCTGGC | (SEQ ID NO:3) |
| ATGGATGGCC CCAAGGTGAA GCAGTGGCCC CTGACTGAGG AGAAGATCAA GGCCCTGGTG | |
| GAAATCTGCA CTGAGATGGA GAAGGAGGGC AAAATCTCCA AGATTGGCCC CGAGAACCCC | |
| TACAACACCC CTGTGTTTGC CATCAAGAAG AAGGACTCCA CCAAGTGGAG GAAGCTGGTG | |
| GACTTCAGGG AGCTGAACAA GAGGACCCAG GACTTCTGGG AGGTGCAGCT GGGCATCCCC | |
| CACCCCGCTG GCCTGAAGAA GAAGAAGTCT GTGACTGTGC TGGCTGTGGG GGATGCCTAC | |
| TTCTCTGTGC CCCTGGATGA GGACTTCAGG AAGTACACTG CCTTCACCAT CCCCTCCATC | |
| AACAATGAGA CCCCTGGCAT CAGGTACCAG TACAATGTGC TGCCCCAGGG CTGGAAGGGC | |
| TCCCCTGCCA TCTTCCAGTC CTCCATGACC AAGATCCTGG AGCCCTTCAG GAAGCAGAAC | |
| CCTGACATTG TGATCTACCA GTACATGGCT GCCCTGTATG TGGGCTCTGA CCTGGAGATT | |
| GGGCAGCACA GGACCAAGAT TGAGGAGCTG AGGCAGCACC TGCTGAGGTG GGGCCTGACC | |
| ACCCCTGACA AGAAGCACCA GAAGGAGCCC CCCTTCCTGT GGATGGGCTA TGAGCTGCAC | |
| CCCGACAAGT GGACTGTGCA GCCCATTGTG CTGCCTGAGA AGGACTCCTG GACTGTGAAT | |
| GACATCCAGA AGCTGGTGGG CAAGCTGAAC TGGGCCTCCC AAATCTACCC TGGCATCAAG | |
| GTGAGGCAGC TGTGCAAGCT GCTGAGGGGC ACCAAGGCCC TGACTGAGGT GATCCCCCTG | |
| ACTGAGGAGG CTGAGCTGGA GCTGGCTGAG AACAGGGAGA TCCTGAAGGA GCCTGTGCAT | |
| GGGGTGTACT ATGACCCCTC CAAGGACCTG ATTGCTGAGA TCCAGAAGCA GGGCCAGGGC | |
| CAGTGGACCT ACCAAATCTA CCAGGAGCCC TTCAAGAACC TGAAGACTGG CAAGTATGCC | |
| AGGATGAGGG GGGCCCACAC CAATGATGTG AAGCAGCTGA CTGAGGCTGT GCAGAAGATC | |
| ACCACTGAGT CCATTGTGAT CTGGGGCAAG ACCCCCAAGT TCAAGCTGCC CATCCAGAAG | |
| GAGACCTGGG AGACCTGGTG GACTGAGTAC TGGCAGGCCA CCTGGATCCC TGAGTGGGAG | |
| TTTGTGAACA CCCCCCCCCT GGTGAAGCTG TGGTACCAGC TGGAGAAGGA GCCCATTGTG | |
| GGGGCTGAGA CCTTCTATGT GGCTGGGGCT GCCAACAGGG AGACCAAGCT GGGCAAGGCT | |
| GGCTATGTGA CCAACAGGGG CAGGCAGAAG GTGGTGACCC TGACTGACAC CACCAACCAG | |
| AAGACTGCCC TCCAGGCCAT CTACCTGGCC CTCCAGGACT CTGGCCTGGA GGTGAACATT | |
| GTGACTGCCT CCCAGTATGC CCTGGGCATC ATCCAGGCCC AGCCTGATCA GTCTGAGTCT | |
| GAGCTGGTGA ACCAGATCAT TGAGCAGCTG ATCAAGAAGG AGAAGGTGTA CCTGGCCTGG | |
| GTGCCTGCCC ACAAGGGCAT TGGGGGCAAT GAGCAGGTGG ACAAGCTGGT GTCTGCTGGC | |
| ATCAGGAAGG TGCTGTTCCT GGATGGCATT GACAAGGCCC AGGATGAGCA TGAGAAGTAC | |
| CACTCCAACT GGAGGGCTAT GGCCTCTGAC TTCAACCTGC CCCCTGTGGT GGCTAAGGAG | |
| ATTGTGGCCT CCTGTGACAA GTGCCAGCTG AAGGGGGAGG CCATGCATGG GCAGGTGGAC | |
| TGCTCCCCTG GCATCTGGCA GCTGGCCTGC ACCCACCTGG AGGGCAAGGT GATCCTGGTG | |
| GCTGTGCATG TGGCCTCCGG CTACATTGAG GCTGAGGTGA TCCCTGCTGA GACAGGCCAG | |
| GAGACTGCCT ACTTCCTGCT GAAGCTGGCT GGCAGGTGGC CTGTGAAGAC CATCCACACT | |
| GCCAATGGCT CCAACTTCAC TGGGGCCACA GTGAGGGCTG CCTGCTGGTG GGCTGGCATC | |
| AAGCAGGAGT TTGGCATCCC CTACAACCCC CAGTCCCAGG GGGTGGTGGC CTCCATGAAC | |
| GTGCAGATGG CTGTGTTCAT CCACAACTTC AAGAGGAAGG GGGGCATCGG GGGCTACTCC | |
| GCTGGGGAGA GGATTGTGGA CATCATTGCC ACAGACATCC AGACCAAGGA GCTCCAGAAG | |
| CAGATCACCA AGATCCAGAA CTTCAGGGTG TACTACAGGG ACTCCAGGAA CCCCCTGTGG | |
| AAGGGCCCTG CCAAGCTGCT GTGGAAGGGG GAGGGGGCTG TGGTGATCCA GGACAACTCT | |
| GACATCAAGG TGGTGCCCAG GAGGAAGGCC AAGATCATCA GGGACTATGG CAAGCAGATG | |
| GCTGGGGATG ACTGTGTGGC CTCCAGGCAG GATGAGGACT AAAGCCCGGG CAGATCT. | |

In order to produce the IA-pol DNA vaccine construction, inactivation of the enzymatic functions was achieved by replacing a total of nine active-site residues from the enzyme subunits with alanine side-chains. As shown in Table 1, all residues that comprise the catalytic triad of the polymerase, namely Asp112, Asp187, and Asp188, were substituted with alanine (Ala) residues (Larder, et al., Nature 1987, 327: 716-717; Larder, et al., 1989, Proc. Natl. Acad. Sci. 1989, 86: 4803-4807). Three additional mutations were introduced at Asp445, Glu480 and Asp500 to abolish RNase H activity (Asp551 was left unchanged in this IA Pol construct), with each residue being substituted for an Ala residue, respectively (Davies, et al., 1991, Science 252:, 88-95; Schatz, et al., 1989, FEBS Lett. 257: 311-314; Mizrahi, et al., 1990, Nucl. Acids. Res. 18: pp. 5359-5353). HIV pol integrase function was abolished through three mutations at Asp626, Asp678 and Glu714. Again, each of these residues has been substituted with an Ala residue (Wiskerchen, et al., 1995, J. Virol. 69: 376-386; Leavitt, et al., 1993, J. Biol. Chem. 268: 2113-2119). Amino acid residue Pro3 of SEQ ID NO: 4 marks the start of the RT gene. The complete amino acid sequence of IA-Pol is disclosed herein as SEQ ID NO: 4, as follows:

| | |
|---|---|
| Met Ala Pro Ile Ser Pro Ile Glu Tbr Val Pro Val Lys Leu Lys Pro | (SEQ ID NO:4) |
| Gly Met Asp Gly Pro Lys Val Lys Gln Trp Pro Leu Thr Glu Glu Lys | |
| Ile Lys Ala Leu Val Glu Ile Cys Tbr Glu Met Glu Lys Glu Gly Lys | |
| Ile Ser Lys Ile Gly Pro Glu Asn Pro Tyr Asn Thr Pro Val Phe Ala | |
| Ile Lys Lys Lys Asp Ser Thr Lys Trp Arg Lys Leu Val Asp Phe Arg | |
| Glu Leu Asn Lys Arg Thr Gln Asp Phe Trp Glu Val Gln Leu Gly Ile | |
| Pro His Pro Ala Gly Leu Lys Lys Lys Lys Ser Val Thr Val Leu Ala | |
| Val Gly Asp Ala Tyr Phe Ser Val Pro Leu Asp Glu Asp Phe Arg Lys | |
| Tyr Thr Ala Phe Thr Ile Pro Ser Ile Asn Asn Glu Thr Pro Gly Ile | |
| Arg Tyr Gln Tyr Asn Val Leu Pro Gln Gly Trp Lys Gly Ser Pro Ala | |
| Ile Phe Gln Ser Ser Met Thr Lys Ile Leu Glu Pro Phe Arg Lys Gln | |
| Asn Pro Asp Ile Val Ile Tyr Gln Tyr Met Ala Ala Leu Tyr Val Gly | |
| Ser Asp Leu Glu Ile Gly Gln His Arg Thr Lys Ile Glu Glu Leu Arg | |
| Gln His Leu Leu Arg Trp Gly Leu Thr Thr Pro Asp Lys Lys His Gln | |
| Lys Glu Pro Pro Phe Leu Trp Met Gly Tyr Glu Leu His Pro Asp Lys | |
| Trp Thr Val Gln Pro Ile Val Leu Pro Glu Lys Asp Ser Trp Thr Val | |
| Asn Asp Ile Gln Lys Leu Val Gly Lys Leu Asn Trp Ala Ser Gln Ile | |
| Tyr Pro Gly Ile Lys Val Arg Gln Leu Cys Lys Leu Leu Arg Gly Thr | |
| Lys Ala Leu Thr Glu Val Ile Pro Leu Thr Glu Glu Ala Glu Leu Glu | |
| Leu Ala Glu Asn Arg Glu Ile Leu Lys Glu Pro Val His Gly Val Tyr | |
| Tyr Asp Pro Ser Lys Asp Leu Ile Ala Glu Ile Gln Lys Gln Gly Gln | |
| Gly Gln Trp Thr Tyr Gln Ile Tyr Gln Glu Pro Phe Lys Asn Leu Lys | |
| Thr Gly Lys Tyr Ala Arg Met Arg Gly Ala His Thr Asn Asp Val Lys | |
| Gln Leu Thr Glu Ala Val Gln Lys Ile Thr Thr Glu Ser Ile Val Ile | |
| Trp Gly Lys Thr Pro Lys Phe Lys Leu Pro Ile Gln Lys Glu Thr Thr | |
| Glu Thr Thr Thr Thr Glu Tyr Thr Gln Ala Thr Thr Ile Pro Glu Thr | |
| Glu Phe Val Asn Thr Pro Pro Leu Val Lys Leu Thr Tyr Gln Leu Glu | |
| Lys Glu Pro Ile Val Gly Ala Glu Thr Phe Tyr Val Ala Gly Ala Ala | |
| Asn Arg Glu Thr Lys Leu Gly Lys Ala Gly Tyr Val Thr Asn Arg Gly | |
| Arg Gln Lys Val Val Thr Leu Thr Asp Thr Thr Asn Gln Lys Thr Ala | |
| Leu Gln Ala Ile Tyr Leu Ala Leu Gln Asp Ser Gly Leu Glu Val Asn | |
| Ile Val Thr Ala Ser Gln Tyr Ala Leu Gly Ile Ile Gln Ala Gln Pro | |
| Asp Gln Ser Glu Ser Glu Leu Val Asn Gln Ile Ile Glu Gln Leu Ile | |
| Lys Lys Glu Lys Val Tyr Leu Ala Thr Val Pro Ala His Lys Gly Ile | |
| Gly Gly Asn Glu Gln Val Asp Lys Leu Val Ser Ala Gly Ile Arg Lys | |
| Val Leu Phe Leu Asp Gly Ile Asp Lys Ala Gln Asp Glu His Glu Lys | |
| Tyr His Ser Asn Trp Arg Ala Met Ala Ser Asp Phe Asn Leu Pro Pro | |
| Val Val Ala Lys Glu Ile Val Ala Ser Cys Asp Lys Cys Gln Leu Lys | |
| Gly Glu Ala Met His Gly Gln Val Asp Cys Ser Pro Gly Ile Trp Gln | |
| Leu Ala Cys Thr His Leu Glu Gly Lys Val Ile Leu Val Ala Val His | |
| Val Ala Ser Gly Tyr Ile Glu Ala Glu Val Ile Pro Ala Glu Thr Gly | |
| Gln Glu Thr Ala Tyr Phe Leu Leu Lys Leu Ala Gly Arg Trp Pro Val | |
| Lys Thr Ile His Thr Ala Asn Gly Ser Asn Phe Thr Gly Ala Thr Val | |
| Arg Ala Ala Cys Trp Trp Ala Gly Ile Lys Gln Glu Phe Gly Ile Pro | |
| Tyr Asn Pro Gln Ser Gln Gly Val Val Ala Ser Met Asn Lys Glu Leu | |
| Lys Lys Ile Ile Gly Gln Val Arg Asp Gln Ala Glu His Leu Lys Thr | |
| Ala Val Gln Met Ala Val Phe Ile His Asn Phe Lys Arg Lys Gly Gly | |
| Ile Gly Gly Tyr Ser Ala Gly Glu Arg Ile Val Asp Ile Ile Ala Thr | |
| Asp Ile Gln Thr Lys Glu Leu Gln Lys Gln Ile Thr Lys Ile Gln Asn | |
| Phe Arg Val Tyr Tyr Arg Asp Ser Arg Asn Pro Leu Trp Lys Gly Pro | |
| Ala Lys Leu Leu Trp Lys Gly Glu Gly Ala Val Val Ile Gln Asp Asn | |
| Ser Asp Ile Lys Val Val Pro Arg Arg Lys Ala Lys Ile Ile Arg Asp | |
| Tyr Gly Lys Gln Met Ala Gly Asp Asp Cys Val Ala Ser Arg Gln Asp | |
| Glu Asp. | |

As noted above, it will be understood that any combination of the mutations disclosed above may be suitable and therefore be utilized as an IA-pol-based vaccine of the present invention. For example, it may be possible to mutate only 2 of the 3 residues within the respective reverse transcriptase, RNase H, and integrase coding regions while still abolishing these enzymatic activities. However, the IA-pol construct described above and disclosed as SEQ ID NO: 3, as well as the expressed protein (SEQ ID NO: 4) is preferred. It is also preferred that at least one mutation be present in each of the three catalytic domains.

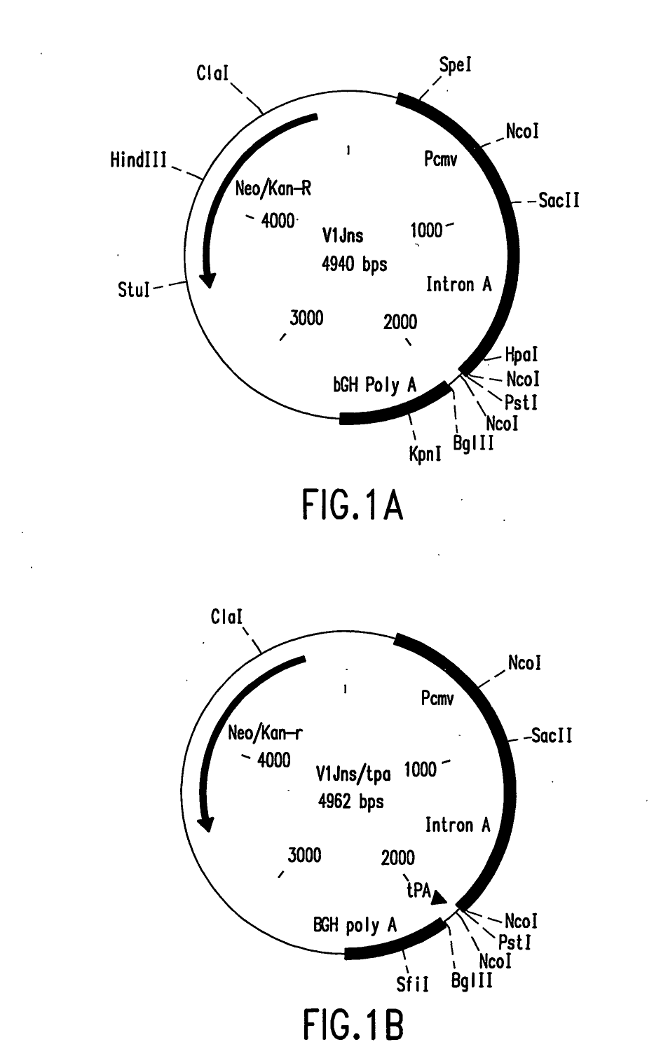

Another aspect of the present invention is to generate codon optimized HIV-1 Pol-based vaccine constructions which comprise a eukaryotic trafficking signal peptide such as from tPA (tissue-type plasminogen activator) or by a leader peptide such as is found in highly expressed mammalian proteins such as immunoglobulin leader peptides. Any functional leader peptide may be tested for efficacy. However, a preferred embodiment of the present invention is to provide for HIV-1 Pol mutant vaccine constructions as disclosed herein which also comprise a leader peptide, preferably a leader peptide from human tPA. In other words, a codon optimized HIV-1 Pol mutant such as IA-Pol (SEQ ID NO: 4) may also comprise a leader peptide at the amino terminal portion of the protein, which may effect cellular trafficking and hence, immunogenicity of the expressed protein within the host cell. As shown in FIGS. 1A-B for the DNA vector V1Jns, a DNA vector which may be utilized to practice the present invention may be modified by known recombinant DNA methodology to contain a leader signal peptide of interest, such that downstream cloning of the modified HIV-1 protein of interest results in a nucleotide sequence which encodes a modified HIV-1 tPA/Pol protein. In the alternative, as noted above, insertion of a nucleotide sequence which encodes a leader peptide may be inserted into a DNA vector housing the open reading frame for the Pol protein of interest. Regardless of the cloning strategy, the end result is a polynucleotide vaccine which comprises vector components for effective gene expression in conjunction with nucleotide sequences which encode a modified HIV-1 Pol protein of interest, including but not limited to a HIV-1 Pol protein which contains a leader peptide. The amino acid sequence of the human tPA leader utilized herein is as follows: MDAMKRGLCCVULLCGAVFVSPSEISS (SEQ ID NO: 28). Therefore, another aspect of the present invention is to generate HIV-1 Pol-based vaccine constructions which comprise a eukaryotic trafficking signal peptide such as from tPA. To this end, the present invention relates to a DNA molecule which encodes a codon optimized wt-pol DNA construct wherein the protease (PR) activity is deleted and a human tPA leader sequence is fused to the 5′ end of the coding region. A DNA molecule which encodes this protein is disclosed herein as SEQ ID NO: 5, the open reading frame disclosed herein as SEQ ID NO: 6.

To this end, the present invention relates to a DNA molecule which encodes a codon optimized wt-pol DNA construct wherein the protease (PR) activity is deleted and a human tPA leader sequence is fused to the 5′ end of the coding region ( herein, “tPA-wt-pol”). A DNA molecule which encodes this protein is disclosed herein as SEQ ID NO: 5, the open reading frame being contained from an initiating Met residue at nucleotides 8-10 to a termination codon from nucleotides 2633-2635. SEQ ID NO: 5 is as follows:

| | |
|---|---|
| GATCACCATG GATGCAATGA AGAGAGGGCT CTGCTGTGTG CTGCTGCTGT GTGGAGCAGT | (SEQ ID NO:5) |
| CTTCGTTTCG CCCAGCGAGA TCTCCGCCCC CATCTCCCCC ATTGAGACTG TGCCTGTGAA | |
| GCTGAAGCCT GGCATGGATG GCCCCAAGGT GAAGCAGTGG CCCCTGACTG AGGAGAAGAT | |
| CAAGGCCCTG GTGGAAATCT GCACTGAGAT GGAGAAGGAG GGCAAAATCT CCAAGATTGG | |
| CCCCGAGAAC CCCTACAACA CCCCTGTGTT TGCCATCAAG AAGAAGGACT CCACCAAGTG | |
| GAGGAAGCTG GTCGACTTCA GGGAGCTGAA CAAGAGGACC CAGGACTTCT GGGAGGTGCA | |
| GCTGGGCATC CCCCACCCCG CTGGCCTGAA GAAGAAGAAG TCTGTGACTG TGCTGGATGT | |
| GGGGGATGCC TACTTCTCTG TGCCCCTGGA TGAGGACTTC AGGAAGTACA CTGCCTTCAC | |
| CATCCCCTCC ATCAACAATG AGACCCCTGG CATCAGGTAC CAGTACAATG TCCTGCCCCA | |
| GGGCTGGAAG GGCTCCCCTG CCATCTTCCA GTCCTCCATG ACCAAGATCC TGGAGCCCTT | |
| CAGGAAGCAG AACCCTGACA TTGTGATCTA CCAGTACATG GATGACCTGT ATGTGGGCTC | |
| TGACCTGGAG ATTGGGCAGC ACAGGACCAA GATTGAGGAG CTGAGGCAGC ACCTGCTGAG | |
| GTGGGGCCTG ACCACCCCTG ACAAGAAGCA CCAGAAGGAG CCCCCCTTCC TGTGGATGGG | |
| CTATGAGCTG CACCCCGACA AGTGGACTGT GCAGCCCATT GTGCTGCCTG AGAAGGACTC | |
| CTGGACTGTG AATGACATCC AGAAGCTGGT GGGCAAGCTG AACTGGQCCT CCCAAATCTA | |
| CCCTGGCATC AAGGTGAGGC AGCTGTGCAA GCTGCTGAGG GGCACCAAGG CCCTCACTGA | |
| GGTGATCCCC CTGACTGAGG AGGCTGAGCT GGAGCTGGCT GAGAACAGGG ACATCCTGAA | |
| GGAGCCTGTG CATGGGGTGT ACTATGACCC CTCCAAGGAC CTGATTGCTG AGATCCAGAA | |
| GCAGGGCCAG GGCCAGTGGA CCTACCAAAT CTACCAGGAC CCCTTCAAGA ACCTGAAGAC | |
| TGGCAAGTAT GCCAGGATGA GGGGGGCCCA CACCAATGAT GTGAAGCAGC TGACTGAGGC | |
| TGTGCAGAAG ATCACCACTG AGTCCATTGT GATCTGGGGC AAGACCCCCA ACTTCAAGCT | |
| CCCCATCCAG AACGAGACCT GGGAGACCTG GTCGACTCAG TACTGGCAGG CCACCTCCAT | |
| CCCTGAGTCG GAGTTTGTCA ACACCCCCCC CCTGCTCAAG CTGTGGTACC AGCTGGAGAA | |
| GGAGCCCATT GTGGGGGCTG AGACCTTCTA TGTGGATGGG GCTGGCAACA GGGAGACCAA | |
| GCTGGGCAAG GCTGGCTATG TGACCAACAG GGGCAGGCAG AAGGTGGTGA CCCTGACTGA | |
| CACCACCAAC CAGAAGACTG AGCTCCACCC CATCTACCTG GCCCTCCACG ACTCTGGCCT | |
| GGAGGTGAAC ATTGTGACTG ACTCCCAGTA TGCCCTGGGC ATCATCCACG CCCAGCCTGA | |
| TCAGTCTGAG TCTCACCTGC TGAACCAGAT CATTGAGCAG CTGATCAAGA AGGAGAAGGT | |
| GTACCTCCCC TGCGTGCCTG CCCACAAGGG CATTCGGGGC AATGAGCAGG TCGACAACCT | |
| GGTGTCTGCT GGCATCACGA AGGTCCTGTT CCTCCATGGC ATTGACAAGG CCCAGGATGA | |
| GCATGAGAAG TACCACTGCA ACTGGAGGGC TATGGCCTCT GACTTCAACC TGCCCCCTGT | |
| GGTGGCTAAC CAGATTGTGG CCTCCTGTGA CAAGTGCCAG CTGAAGGGGG AGGCCATGCA | |
| TGGGCAGGTG GACTGCTCCC CTGGCATCTG GCAGCTGGAC TGCACCCACC TGGAGGGCAA | |
| GGTGATCCTG GTGGCTGTGC ATGTGGCCTC CGGCTACATT GAGGCTGAGG TGATCCCTGC | |
| TGAGACAGGC CAGGAGACTG CCTACTTCCT GCTGAACCTG GCTGGCAGGT GGCCTGTGAA | |
| GACCATCCAC ACTGACAATG GCTCCAACTT CACTCGGGCC ACAGTGACCG CTGCCTCCTG | |
| GTGGCCTGGC ATCAAGCAGG AGTTTGCCAT CCCCTACAAC CCCCAGTCCC AGGGGGTGGT | |
| GGAGTCCATG AACAAGGAGC TGAACAAGAT CATTGGGCAG GTGAGGGACC AGGCTGAGCA | |
| CCTGAAGACA GCTGTGCAGA TQGCTGTGTT CATCCACAAC TTCAAGAGGA AGGGGGGGAT | |
| CGGGGGCTAC TCCGCTGGGG AGAGGATTGT GGACATCATT GCCACAGACA TCCAGACCAA | |
| GGAGGTGCAG AAGCAGATCA CCAAGATCCA GAACTTCAGG GTGTACTACA GGGACTCCAG | |
| GAACCCCCTG TGGAAGGGCC CTGCCAAGCT GCTGTGGAAG GGGGAGGGGG CTGTGGTGAT | |
| CCAGGACAAC TCTGACATCA AGGTGGTGCC CAGGAGGAAG GCCAAGATCA TCAGGGACTA | |
| TGGCAAGCAG ATGGCTGGGG ATGACTGTGT GGCCTCCAGG CAGGATGAGG ACTAAAGCCC | |
| GGGCAGATCT. | |

The open reading frame of the wild type tPA-pol construct disclosed as SEQ ID NO: 5 contains 875 amino acids, disclosed herein as SEQ ID NO: 6, as follows:

| | |
|---|---|
| Met Asp Ala Met Lys Arg Gly Leu Cys Cys Val Leu Leu Leu Cys Gly | (SEQ ID NO:6) |
| Ala Val Phe Val Ser Pro Ser Glu Ile Ser Ala Pro Ile Ser Pro Ile | |
| Glu Thr Val Pro Val Lys Leu Lys Pro Gly Met Asp Gly Pro Lys Val | |
| Lys Gln Trp Pro Leu Thr Glu Glu Lys Ile Lys Ala Leu Val Glu Ile | |
| Cys Thr Glu Met Glu Lys Glu Gly Lys Ile Ser Lys Ile Gly Pro Glu | |
| Asn Pro Tyr Asn Thr Pro Val Phe Ala Ile Lys Lys Lys Asp Ser Thr | |
| Lys Trp Arg Lys Leu Val Asp Phe Arg Glu Leu Asn Lys Arg Thr Gln | |
| Asp Phe Trp Glu Val Gln Leu Gly Ile Pro His Pro Ala Gly Leu Lys | |
| Lys Lys Lys Ser Val Thr Val Leu Asp Val Gly Asp Ala Tyr Phe Ser | |
| Val Pro Leu Asp Glu Asp Phe Arg Lys Tyr Thr Ala Phe Thr Ile Pro | |
| Ser Ile Asn Asn Glu Thr Pro Gly Ile Arg Tyr Gln Tyr Asn Val Leu | |
| Pro Gln Gly Trp Lys Gly Ser Pro Ala Ile Phe Gln Ser Ser Met Thr | |
| Lys Ile Leu Glu Pro Phe Arg Lys Gln Asn Pro Asp Ile Val Ile Tyr | |
| Gln Tyr Met Asp Asp Leu Tyr Val Gly Ser Asp Leu Glu Ile Gly Gln | |
| His Arg Thr Lys Ile Glu Glu Leu Arg Gln His Leu Leu Arg Trp Gly | |
| Leu Thr Thr Pro Asp Lys Lys His Gln Lys Glu Pro Pro Phe Leu Trp | |
| Met Gly Tyr Glu Leu His Pro Asp Lys Trp Thr Val Gln Pro Ile Val | |
| Leu Pro Glu Lys Asp Ser Trp Thr Val Asn Asp Ile Gln Lys Leu Val | |
| Gly Lys Leu Asn Trp Ala Ser Gln Ile Tyr Pro Gly Ile Lys Val Arg | |
| Gln Leu Cys Lys Leu Leu Arg Gly Thr Lys Ala Leu Thr Glu Val Ile | |
| Pro Leu Thr Glu Glu Ala Glu Leu Glu Leu Ala Glu Asn Arg Glu Ile | |
| Leu Lys Glu Pro Val His Gly Val Tyr Tyr Asp Pro Ser Lys Asp Leu | |
| Ile Ala Glu Ile Gln Lys Gln Gly Gln Gly Gln Trp Thr Tyr Gln Ile | |
| Tyr Gln Glu Pro Phe Lys Asn Leu Lys Thr Gly Lys Tyr Ala Arg Met | |
| Arg Gly Ala His Thr Asn Asp Val Lys Gln Leu Thr Glu Ala Val Gln | |
| Lys Ile Thr Thr Glu Ser Ile Val Ile Trp Gly Lys Thr Pro Lys Phe | |
| Lys Leu Pro Ile Gln Lys Glu Thr Trp Glu Thr Trp Trp Thr Glu Tyr | |
| Trp Gln Ala Thr Trp Ile Pro Glu Trp Glu Phe Val Asn Thr Pro Pro | |
| Leu Val Lys Leu Trp Tyr Gln Leu Glu Lys Glu Pro Ile Val Gly Ala | |
| Glu Thr Phe Tyr Val Asp Gly Ala Ala Asn Arg Glu Thr Lys Leu Gly | |
| Lys Ala Gly Tyr Val Thr Asn Arg Gly Arg Gln Lys Val Val Thr Leu | |
| Thr Asp Thr Thr Asn Gln Lys Thr Glu Leu Gln Ala Ile Tyr Leu Ala | |
| Leu Gln Asp Ser Gly Leu Glu Val Asn Ile Val Thr Asp Ser Gln Tyr | |
| Ala Leu Gly Ile Ile Gln Ala Gln Pro Asp Gln Ser Glu Ser Glu Leu | |
| Val Asn Gln Ile Ile Glu Gln Leu Ile Lys Lys Glu Lys Val Tyr Leu | |
| Ala Trp Val Pro Ala His Lys Gly Ile Gly Gly Asn Glu Gln Val Asp | |
| Lys Leu Val Ser Ala Gly Ile Arg Lys Val Leu Phe Leu Asp Gly Ile | |
| Asp Lys Ala Gln Asp Glu His Glu Lys Tyr His Ser Asn Trp Arg Ala | |
| Met Ala Ser Asp Phe Asn Leu Pro Pro Val Val Ala Lys Glu Ile Val | |
| Ala Ser Cys Asp Lys Cys Gln Leu Lys Gly Glu Ala Met His Gly Gln | |
| Val Asp Cys Ser Pro Gly Ile Trp Gln Leu Asp Cys Thr His Leu Glu | |
| Gly Lys Val Ile Leu Val Ala Val His Val Ala Ser Gly Tyr Ile Glu | |
| Ala Glu Val Ile Pro Ala Glu Thr Gly Gln Glu Thr Ala Tyr Phe Leu | |
| Leu Lys Leu Ala Gly Arg Trp Pro Val Lys Thr Ile His Thr Asp Asn | |
| Gly Ser Asn Phe Thr Gly Ala Thr Val Arg Ala Ala Cys Trp Trp Ala | |
| Gly Ile Lys Gln Glu Phe Gly Ile Pro Tyr Asn Pro Gln Ser Gln Gly | |
| Val Val Glu Ser Met Asn Lys Glu Leu Lys Lys Ile Ile Gly Gln Val | |
| Arg Asp Gln Ala Glu His Leu Lys Thr Ala Val Gln Met Ala Val Phe | |
| Ile His Asn Phe Lys Arg Lys Gly Gly Ile Gly Gly Tyr Ser Ala Gly | |
| Glu Arg Ile Val Asp Ile Ile Ala Thr Asp Ile Gln Thr Lys Glu Leu | |
| Gln Lys Gln Ile Thr Lys Ile Gln Asn Phe Arg Val Tyr Tyr Arg Asp | |
| Ser Arg Asn Pro Leu Trp Lys Gly Pro Ala Lys Leu Leu Trp Lys Gly | |
| Glu Gly Ala Val Val Ile Gln Asp Asn Ser Asp Ile Lys Val Val Pro | |
| Arg Arg Lys Ala Lys Ile Ile Arg Asp Tyr Gly Lys Gln Met Ala Gly | |
| Asp Asp Cys Val Ala Ser Arg Gln Asp Glu Asp. | |

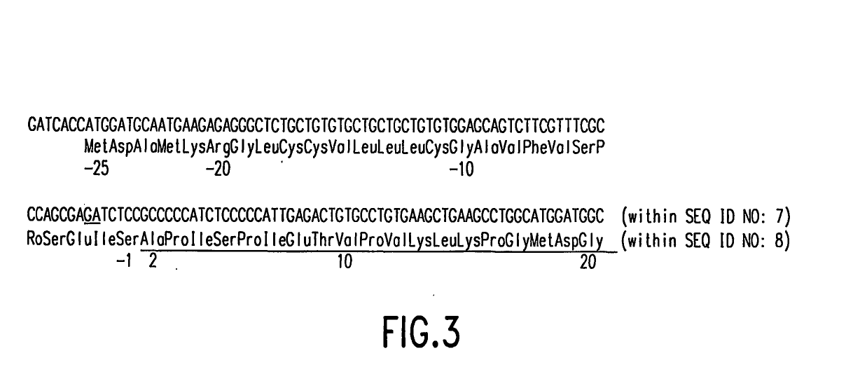

The present invention also relates to a codon optimized HIV-1 Pol mutant such as IA-Pol (SEQ ID NO: 4) which comprises a leader peptide at the amino terminal portion of the protein, which may effect cellular trafficking and hence, immunogenicity of the expressed protein within the host cell. Any such HIV-1 DNA pol mutant disclosed in the above paragraphs is suitable for fusion downstream of a leader peptide, such as a leader peptide including but not limited to the human tPA leader sequence. Therefore, any such leader peptide-based HIV-1 pol mutant construct may include but is not limited to a mutated DNA molecule which effectively alters the catalytic activity of the RT, RNase and/or IN region of the expressed protein, resulting in at least substantially decreased enzymatic activity one or more of the RT, RNase H and/or IN functions of HIV-1 Pol. In a preferred embodiment of this portion of the invention, a leader peptide/HIV-1 DNA pol construct contains a mutation or mutations within the Pol coding region which effectively abolishes RT, RNase H and IN activity. An especially preferable HIV-1 DNA pol construct is a DNA molecule which contains at least one point mutation which alters the active site and catalytic activity within the RT, RNase H and IN domains of Pol, such that each activity is at least substantially abolished, and preferably totally abolished. Such a HIV-1 Pol mutant will most likely comprise at least one point mutation in or around each catalytic domain responsible for RT, RNase H and IN activity, respectfully. An especially preferred embodiment of this portion of the invention relates to a human tPA leader fused to the IA-Pol protein comprising the nine mutations shown in Table 1. The DNA molecule is disclosed herein as SEQ ID NO: 7 and the expressed tPA-IA Pol protein comprises a fusion junction as shown in FIG. 3. The complete amino acid sequence of the expressed protein is set forth in SEQ ID NO: 8. To this end, SEQ ID NO: 7 discloses the nucleotide sequence which codes for a human tPA leader fused to the IA Pol protein comprising the nine mutations shown in Table 1 (herein, “tPA-opt-IApol”). The open reading frame begins with the initiating Met (nucleotides 8-10) and terminates with a “TAA” codon at nucleotides 2633-2635. The nucleotide sequence encoding tPA-IAPol is also disclosed as follows:

| | |
|---|---|
| GATCACCATG GATGCAATGA AGAGAGGGCT CTGCTGTGTG CTGCTGCTGT GTGGAGCAGT | (SEQ ID NO:7) |
| CTTCGTTTCG CCCAGCGAGA TCTCCGCCCC CATCTCCCCC ATTGAGACTG TGCCTGTGAA | |
| GCTGAAGCCT GGCATGGATG GCCCCAAGGT GAAGCAGTGG CCCCTGACTG AGGAGAAGAT | |
| CAAGGCCCTG GTGGAAATCT GCACTGAGAT GGAGAAGGAC GGCAAAATCT CCAAGATTGG | |
| CCCCGAGAAC CCCTACAACA CCCCTGTQTT TGCCATCAAG AAGAAGGACT CCACCAAGTG | |
| GAGGAAGCTG GTGGACTTCA GGGAGCTGAA CAAGAGGACC CAGGACTTCT GGGAGGTGCA | |
| GCTGGGCATC CCCCACCCCG CTGGCCTGAA GAAGAAGAAG TCTGTGACTG TGCTGGCTGT | |
| GGGGGATGCC TACTTCTCTG TGCCCCTGGA TGAGGACTTC AGGAAGTACA CTGCCTTCAC | |
| CATCCCCTCC ATCAACAATG AGACCCCTGG CATCAGGTAC CAGTACAATG TGCTGCCCCA | |
| GGGCTGGAAG GGCTCCCCTG CCATCTTCCA GTCCTCCATG ACCAAGATCC TGGAGCCCTT | |
| CACGAAGCAG AACCCTGACA TTGTGATCTA CCAGTACATG GCTGCCCTGT ATGTGGGCTC | |
| TGACCTGGAG ATTGGGCAGC ACAGGACCAA GATTGAGGAG CTGAGGCAGC ACCTGCTGAG | |
| GTGCGGCCTG ACCACCCCTG ACAAGAAGCA CCAGAAGGAG CCCCCCTTCC TGTGGATGGG | |
| CTATGAGCTG CACCCCGACA AGTGGACTGT GCAGCCCATT GTGCTGCCTG AGAAGGACTC | |
| CTGGACTGTG AATGACATCC AGAAGCTGGT GGGCAAGCTG AACTGGGCCT CCCAAATCTA | |
| CCCTGGCATC AAGGTGAGGC AGCTGTGCAA GCTGCTGAGG GGCACCAAGG CCCTGACTGA | |
| GGTGATCCCC CTGACTGAGG AGGCTGAGCT GGAGCTCGCT GAGAACAGGG AGATCCTGAA | |
| GGAGCCTGTG CATGGGGTCT ACTATGACCC CTCCAAGGAC CTGATTGCTG AGATCCAGAA | |
| GCAGGGCCAG GGCCAGTGGA CCTACCAAAT CTACCAGGAG CCCTTCAAGA ACCTGAAGAC | |
| TGGCAAGTAT GCCAGGATGA GGGGGGCCCA CACCAATGAT GTGAAGCAGC TGACTGAGGC | |
| TGTGCAGAAG ATCACCACTG AGTCCATTGT GATGTGGGGG AAGACCCCCA AGTTCAAGCT | |
| GCCCATCCAG AAGGAGACCT GGGAGACCTG GTGGACTGAG TACTGGCAGG CCACCTGGAT | |
| CCCTGAGTGG GAGTTTGTGA ACACCCCCCC CCTGGTGAAG CTGTCGTACC AGCTGGAGAA | |
| GGAGCCCATT GTGGGGGCTG AGACCTTCTA TGTGGCTCGG GCTGCCAACA GGGAGACCAA | |
| GCTGGGCAAG GCTGGCTATG TGACCAACAG GGGCAGGGAG AAGGTGGTGA CCCTGACTGA | |
| CACCACCAAC CAGAAGACTG CCCTCCAGGC CATCTACCTG GCCCTCCAGG ACTCTGGCCT | |
| GGAGGTGAAC ATTGTGACTG CCTCCCAGTA TGCCCTGGGC ATCATCCAGG CCCAGCCTGA | |
| TCAGTCTGAG TCTGAGCTGG TGAACCAGAT CATTGAGCAG CTGATCAAGA AGGAGAAGGT | |
| GTACCTGGCC TGGGTGCCTG CCCACAAGGG CATTGGGGCC AATGAGCAGG TGGACAAGCT | |
| GGTGTCTGCT GGCATCAGGA AGGTGCTGTT CCTGGATGGC ATTGACAAGG CCCAGGATGA | |
| GCATGAGAAG TACCACTCCA ACTGGAGGGC TATGGCCTCT GACTTCAACC TGCCCCCTGT | |
| GGTGGCTAAG GAGATTGTGG CCTCCTGTGA CAAGTGCCAG CTGAAGGGGG AGGCCATGCA | |
| TGGGCAGGTG GACTGCTCCC CTGGCATCTG GCAGCTGGCC TGCACCCACC TGGAGGGCAA | |
| GGTGATCCTG GTGGCTGTGC ATGTGGCCTC CGGCTACATT GAGGCTGAGG TGATCCCTGC | |
| TGAGACAGGC CAGGAGACTG CCTACTTCCT GCTGAAGCTG GCTGGCAGGT GGCCTGTGAA | |
| GACCATCCAC ACTGCCAATG GCTCCAACTT CACTGGGGCC ACAGTGAGGG CTGCCTGCTG | |
| GTGGGCTGGC ATCAAGCAGG AGTTTGGCAT CCCCTACAAC CCCCAGTCCC AGGGGGTGGT | |
| GGCCTCCATG AACAAGGAGC TGAAGAAGAT CATTGGGCAG GTGAGGGACC AGGCTGAGCA | |
| CCTGAAGACA GCTGTGCAGA TGGCTGTGTT CATCCACAAC TTCAAGAGGA AGGGGGCCAT | |
| CGGGGGCTAC TCCGCTGGGG AGAGGATTGT GGACATCATT GCCACAGACA TCCAGACCAA | |
| GGAGCTCCAG AAGCAGATCA CCAAGATCCA GAACTTCAGG GTGTACTACA GGGACTCCAG | |
| GAACCCCCTG TGGAAGGGCC CTGCCAAGCT GCTGTGGAAG GGGGAGGGGG CTGTGGTGAT | |
| CCAGGACAAC TCTGACATCA AGGTGGTGCC CAGGAGGAAG GCCAAGATCA TCAGGGACTA | |
| TGGGAAGCAG ATGGCTGGGG ATGACTGTGT GGCCTCCAGG CAGGATGAGG ACTAAAGCCC | |
| GGGCAGATCT. | |

The open reading frame of the tPA-IA-pol construct disclosed as SEQ D NO: 7 contains 875 amino acids, disclosed herein as tPA-IA-Pol and SEQ ID NO: 8, as follows:

| | |
|---|---|
| Met Asp Ala Met Lys Arg Gly Leu Cys Cys Val Leu Leu Leu Cys Gly | (SEQ ID NO:8) |
| Ala Val Phe Val Ser Pro Ser Glu Ile Ser Ala Pro Ile Ser Pro Ile | |
| Glu Thr Val Pro Val Lys Leu Lys Pro Gly Met Asp Gly Pro Lys Val | |
| Lys Gln Trp Pro Leu Thr Glu Glu Lys Ile Lys Ala Leu Val Glu Ile | |
| Cys Thr Glu Met Glu Lys Glu Gly Lys Ile Ser Lys Ile Gly Pro Glu | |
| Asn Pro Tyr Asn Thr Pro Val Phe Ala Ile Lys Lys Lys Asp Ser Thr | |
| Lys Trp Arg Lys Leu Val Asp Phe Arg Glu Leu Asn Lys Arg Thr Gln | |
| Asp Phe Trp Glu Val Gln Leu Gly Ile Pro His Pro Ala Gly Leu Lys | |
| Lys Lys Lys Ser Val Thr Val Leu Ala Val Gly Asp Ala Tyr Phe Ser | |
| Val Pro Leu Asp Glu Asp Phe Arg Lys Tyr Thr Ala Phe Thr Ile Pro | |
| Ser Ile Asn Asn Glu Thr Pro Gly Ile Arg Tyr Gln Tyr Asn Val Leu | |
| Pro Gln Gly Trp Lys Gly Ser Pro Ala Ile Phe Gln Ser Ser Met Thr | |
| Lys Ile Leu Glu Pro Phe Arg Lys Gln Asn Pro Asp Ile Val Ile Tyr | |
| Gln Tyr Met Ala Ala Leu Tyr Val Gly Ser Asp Leu Glu Ile Gly Gln | |
| His Arg Thr Lys Ile Glu Glu Leu Arg Gln His Leu Leu Arg Trp Gly | |
| Leu Thr Thr Pro Asp Lys Lys His Gln Lys Glu Pro Pro Phe Leu Trp | |
| Met Gly Tyr Glu Leu His Pro Asp Lys Trp Thr Val Gln Pro Ile Val | |
| Leu Pro Glu Lys Asp Ser Trp Thr Val Asn Asp Ile Gln Lys Leu Val | |
| Gly Lys Leu Asn Trp Ala Ser Gln Ile Tyr Pro Gly Ile Lys Val Arg | |
| Gln Leu Cys Lys Leu Leu Arg Gly Thr Lys Ala Leu Thr Glu Val Ile | |
| Pro Leu Thr Glu Glu Ala Glu Leu Glu Leu Ala Glu Asn Arg Glu Ile | |
| Leu Lys Glu Pro Val His Gly Val Tyr Tyr Asp Pro Ser Lys Asp Leu | |
| Ile Ala Glu Ile Gln Lys Gln Gly Gln Gly Gln Trp Thr Tyr Gln Ile | |
| Tyr Gln Glu Pro Phe Lys Asn Leu Lys Thr Gly Lys Tyr Ala Arg Met | |
| Arg Gly Ala His Thr Asn Asp Val Lys Gln Leu Thr Glu Ala Val Gln | |
| Lys Ile Thr Thr Glu Ser Ile Val Ile Trp Gly Lys Thr Pro Lys Phe | |
| Lys Leu Pro Ile Gln Lys Glu Thr Trp Glu Thr Trp Trp Thr Glu Tyr | |
| Trp Gln Ala Thr Trp Ile Pro Glu Trp Glu Phe Val Asn Thr Pro Pro | |
| Leu Val Lys Leu Trp Tyr Gln Leu Glu Lys Glu Pro Ile Val Gly Ala | |
| Glu Thr Phe Tyr Val Ala Gly Ala Ala Asn Arg Glu Thr Lys Leu Gly | |
| Lys Ala Gly Tyr Val Thr Asn Arg Gly Arg Gln Lys Val Val Thr Leu | |
| Thr Asp Thr Thr Asn Gln Lys Thr Ala Leu Gln Ala Ile Tyr Leu Ala | |
| Leu Gln Asp Ser Gly Leu Glu Val Asn Ile Val Thr Ala Ser Gln Tyr | |
| Ala Leu Gly Ile Ile Gln Ala Gln Pro Asp Gln Ser Glu Ser Glu Leu | |
| Val Asn Gln Ile Ile Glu Gln Leu Ile Lys Lys Glu Lys Val Tyr Leu | |
| Ala Trp Val Pro Ala His Lys Gly Ile Gly Gly Asn Glu Gln Val Asp | |
| Lys Leu Val Ser Ala Gly Ile Arg Lys Val Leu Phe Leu Asp Gly Ile | |
| Asp Lys Ala Gln Asp Glu His Glu Lys Tyr His Ser Asn Trp Arg Ala | |
| Met Ala Ser Asp Phe Asn Leu Pro Pro Val Val Ala Lys Glu Ile Val | |
| Ala Ser Cys Asp Lys Cys Gln Leu Lys Gly Glu Ala Met His Gly Gln | |
| Val Asp Cys Ser Pro Gly Ile Trp Gln Leu Ala Cys Thr His Leu Glu | |
| Gly Lys Val Ile Leu Val Ala Val His Val Ala Ser Gly Tyr Ile Glu | |
| Ala Glu Val Ile Pro Ala Glu Thr Gly Gln Glu Thr Ala Tyr Phe Leu | |
| Leu Lys Leu Ala Gly Arg Trp Pro Val Lys Thr Ile His Thr Ala Asn | |
| Gly Ser Asn Phe Thr Gly Ala Thr Val Arg Ala Ala Cys Trp Trp Ala | |
| Gly Ile Lys Gln Glu Phe Gly Ile Pro Tyr Asn Pro Gln Ser Gln Gly | |
| Val Val Ala Ser Met Asn Lys Glu Leu Lys Lys Ile Ile Gly Gln Val | |
| Arg Asp Gln Ala Glu His Leu Lys Thr Ala Val Gln Met Ala Val Phe | |
| Ile His Asn Phe Lys Arg Lys Gly Gly Ile Gly Gly Tyr Ser Ala Gly | |
| Glu Arg Ile Val Asp Ile Ile Ala Thr Asp Ile Gln Thr Lys Glu Leu | |
| Gln Lys Gln Ile Thr Lys Ile Gln Asn Phe Arg Val Tyr Tyr Arg Asp | |
| Ser Arg Asn Pro Leu Trp Lys Gly Pro Ala Lys Leu Leu Trp Lys Gly | |
| Glu Gly Ala Val Val Ile Gln Asp Asn Ser Asp Ile Lys Val Val Pro | |
| Arg Arg Lys Ala Lys Ile Ile Arg Asp Tyr Gly Lys Gln Met Ala Gly | |
| Asp Asp Cys Val Ala Ser Arg Gln Asp Glu Asp. | |

The present invention also relates to a substantially purified protein expressed from the DNA polynucleotide vaccines of the present invention, especially the purified proteins set forth below as SEQ ID NOs: 2, 4, 6, and 8. These purified proteins may be useful as protein-based HIV vaccines.

The DNA backbone of the DNA vaccines of the present invention are preferably DNA plasmid expression vectors. DNA plasmid expression vectors are well known in the art and the present DNA vector vaccines may be comprised of any such expression backbone which contains at least a promoter for RNA polymerase transcription, and a transcriptional terminator 3′ to the HIV pol coding sequence. In one preferred embodiment, the promoter is the Rous sarcoma virus (RSV) long terminal repeat (LTR) which is a strong transcriptional promoter. A more preferred promoter is the cytomegalovirus promoter with the intron A sequence (CMV-intA). A preferred transcriptional terminator is the bovine growth hormone terminator. In addition, to assist in large scale preparation of an HIV pol DNA vector vaccine, an antibiotic resistance marker is also preferably included in the expression vector. Ampicillin resistance genes, neomycin resistance genes or any other pharmaceutically acceptable antibiotic resistance marker may be used. In a preferred embodiment of this invention, the antibiotic resistance gene encodes a gene product for neomycin resistance. Further, to aid in the high level production of the pharmaceutical by fermentation in prokaryotic organisms, it is advantageous for the vector to contain an origin of replication and be of high copy number. Any of a number of commercially available prokaryotic cloning vectors provide these benefits. In a preferred embodiment of this invention, these functionalities are provided by the commercially available vectors known as pUC. It is desirable to remove non-essential DNA sequences. Thus, the lacZ and lacI coding sequences of pUC are removed in one embodiment of the invention.

DNA expression vectors which exemplify but in no way limit the present invention are disclosed in PCT International Application No. PCT/US94/02751, International Publication No. WO 94/21797, hereby incorporated by reference. A first DNA expression vector is the expression vector pnRSV, wherein the rous sarcoma virus (RSV) long terminal repeat (LTR) is used as the promoter. A second embodiment relates to plasmid V1, a mutated pBR322 vector into which the CMV promoter and the BGH transcriptional terminator is cloned. Another embodiment regarding DNA vector backbones relates to plasmid V1J. Plasmid V1J is derived from plasmid V1 and removes promoter and transcription termination elements in order to place them within a more defined context, create a more compact vector, and to improve plasmid purification yields. Therefore, V1J also contains the CMVintA promoter and (BGH) transcription termination elements which control the expression of the HIV pol-based genes disclosed herein. The backbone of V1J is provided by pUC18. It is known to produce high yields of plasmid, is well-characterized by sequence and function, and is of minimum size. The entire lac operon was removed and the remaining plasmid was purified from an agarose electrophoresis gel, blunt-ended with the T4 DNA polymerase, treated with calf intestinal alkaline phosphatase, and ligated to the CMVintA/BGH element. In a preferred DNA expression vector, the ampicillin resistance gene is removed from V1J and replaced with a neomycin resistance gene, to generate V1Jneo. An especially preferred DNA expression vector is V1Jns, which is the same as V1J except that a unique Sfi1 restriction site has been engineered into the single Kpn1 site at position 2114 of V1J-neo. The incidence of Sfi1 sites in human genomic DNA is very low (approximately 1 site per 100,000 bases). Thus, this vector allows careful monitoring for expression vector integration into host DNA, simply by Sfi1 digestion of extracted genomic DNA. Yet another preferred DNA expression vector used as the backbone to the HIV-1 pol-based DNA vaccines of the present invention is V1R. In this vector, as much non-essential DNA as possible is “trimmed” from the vector to produce a highly compact vector. This vector is a derivative of V1Jns. This vector allows larger inserts to be used, with less concern that undesirable sequences are encoded and optimizes uptake by cells when the construct encoding specific influenza virus genes is introduced into surrounding tissue. The specific DNA vectors of the present invention include but are not limited to V1, V1J (SEQ ID NO: 13), V1Jneo (SEQ ID NO: 14), V1Jns (FIG. 1A, SEQ ID NO: 15), V1R (SEQ ID NO: 26), and any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO: 28.

The present invention especially relates to a DNA vaccine and a pharmaceutically active vaccine composition which contains this DNA vaccine, and the use as prophylactic and/or therapeutic vaccine for host immunization, preferably human host immunization, against an HIV infection or to combat an existing HIV condition. These DNA vaccines are represented by codon optimized DNA molecules encoding HIV-1 Pol or biologically active Pol modifications or Pol-containing fusion proteins which are ligated within an appropriate DNA plasmid vector, with or without a nucleotide sequence encoding a functional leader peptide. DNA vaccines of the present invention may comprise codon optimized DNA molecules encoding HIV-1 Pol or biologically active Pol modifications or Pol-containing fusion proteins ligated in DNA vectors V1, V1J (SEQ ID NO: 14), V1Jneo (SEQ ID NO: 15), V1Jns (FIG. 1A, SEQ ID NO: 16), V1R (SEQ ID NO: 26), or any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO: 28. To this end, polynucleotide vaccine constructions include, V1Jns-wt-pol and V1R-wtpol (comprising the DNA molecule encoding WT Pol, as set forth in SEQ ID NO: 2), V1Jns-tPA-WTPol, (comprising the DNA molecule encoding tPA Pol, as set forth in SEQ ID NO: 6), V1Jns-IAPol (comprising the DNA molecule encoding IA Pol, as set forth in SEQ ID NO: 4), and V1Jns-tPA-IAPol, (comprising the DNA molecule encoding tPA-IA Pol, as set forth in SEQ ID NO: 8). Polynucleotide vaccine constructions V1R-wtpol, V1Jns-IAPol, and V1Jns-tPA-IAPol, are exemplified in Example Sections 3-5.

It will be evident upon review of the teaching within this specification that numerous vector/Pol antigen constructs may be generated. While the exemplified constructs are preferred, any number of vector/Pol antigen combinations are within the scope of the present invention, especially wild type or modified/inactivated Pol proteins which comprise at least one, preferably 5 or more and especially all nine mutations as shown in Table 1, with or without the inclusion of a leader sequence such as human tPA.

The DNA vector vaccines of the present invention may be formulated in any pharmaceutically effective formulation for host administration. Any such formulation may be, for example, a saline solution such as phosphate buffered saline (PBS). It will be useful to utilize pharmaceutically acceptable formulations which also provide long-term stability of the DNA vector vaccines of the present invention. During storage as a pharmaceutical entity, DNA plasmid vaccines undergo a physiochemical change in which the supercoiled plasmid converts to the open circular and linear form. A variety of storage conditions (low pH, high temperature, low ionic strength) can accelerate this process. Therefore, the removal and/or chelation of trace metal ions (with succinic or malic acid, or with chelators containing multiple phosphate ligands) from the DNA plasmid solution, from the formulation buffers or from the vials and closures, stabilizes the DNA plasmid from this degradation pathway during storage. In addition, inclusion of non-reducing free radical scavengers, such as ethanol or glycerol, are useful to prevent damage of the DNA plasmid from free radical production that may still occur, even in apparently demetalated solutions. Furthermore, the buffer type, pH, salt concentration, light exposure, as well as the type of sterilization process used to prepare the vials, may be controlled in the formulation to optimize the stability of the DNA vaccine. Therefore, formulations that will provide the highest stability of the DNA vaccine will be one that includes a demetalated solution containing a buffer (phosphate or bicarbonate) with a pH in the range of 7-8, a salt (NaCl, KCl or LiCl) in the range of 100-200 mM, a metal ion chelator (e.g., EDTA, diethylenetriaminepenta-acetic acid (DTPA), malate, inositol hexaphosphate, tripolyphosphate or polyphosphoric acid), a non-reducing free radical scavenger (e.g. ethanol, glycerol, methionine or dimethyl sulfoxide) and the highest appropriate DNA concentration in a sterile glass vial, packaged to protect the highly purified, nuclease free DNA from light. A particularly preferred formulation which will enhance long term stability of the DNA vector vaccines of the present invention would comprise a Tris-HCl buffer at a pH from about 8.0 to about 9.0; ethanol or glycerol at about 3% w/v; EDTA or DTPA in a concentration range up to about 5 mM; and NaCl at a concentration from about 50 mM to about 500 mM. The use of such stabilized DNA vector vaccines and various alternatives to this preferred formulation range is described in detail in PCT International Application No. PCT/US97/06655 and PCT International Publication No. WO 97/40839, both of which are hereby incorporated by reference.

The DNA vector vaccines of the present invention may also be formulated with an adjuvant or adjuvants which may increase immunogenicity of the DNA polynucleotide vaccines of the present invention. A number of these adjuvants are known in the art and are available for use in a DNA vaccine, including but not limited to particle bombardment using DNA-coated gold beads, co-administration of DNA vaccines with plasmid DNA expressing cytokines, chemokines, or costimulatory molecules, formulation of DNA with cationic lipids or with experimental adjuvants such as saponin, monophosphoryl lipid A or other compounds which increase immunogenicity of the DNA vaccine. Another adjuvant for use in the DNA vector vaccines of the present invention are one or more forms of an aluminum phosphate-based adjuvant wherein the aluminum phosphate-based adjuvant possesses a molar PO₄/Al ratio of approximately 0.9. An additional mineral-based adjuvant may be generated from one or more forms of a calcium phosphate. These mineral-based adjuvants are useful in increasing cellular and humoral responses to DNA vaccination. These mineral-based compounds for use as DNA vaccines adjuvants are disclosed in PCT International Application No. PCT/US98/02414, PCT International Publication No. WO 98/35562, which is hereby incorporated by reference. Another preferred adjuvant is a non-ionic block copolymer which shows adjuvant activity with DNA vaccines. The basic structure comprises blocks of polyoxyethylene (POE) and polyoxypropylene (POP) such as a POE-POP-POE block copolymer. Newman et al. (1998, Critical Reviews in Therapeutic Drug Carrier Systems 15(2): 89-142) review a class of non-ionic block copolymers which show adjuvant activity. The basic structure comprises blocks of polyoxyethylene (POE) and polyoxypropylene (POP) such as a POE-POP-POE block copolymer. Newman et al. id., disclose that certain POE-POP-POE block copolymers may be useful as adjuvants to an influenza protein-based vaccine, namely higher molecular weight POE-POP-POE block copolymers containing a central POP block having a molecular weight of over about 9000 daltons to about 20,000 daltons and flanking POE blocks which comprise up to about 20% of the total molecular weight of the copolymer (see also U.S. Reissue Pat. No. 36,665, U.S. Pat. No. 5,567,859, U.S. Pat. No. 5,691,387, U.S. Pat. No. 5,696,298 and U.S. Pat. No. 5,990,241, all issued to Emanuele, et al., regarding these POE-POP-POE block copolymers). WO 96/04932 further discloses higher molecular weight POE/POP block copolymers which have surfactant characteristics and show biological efficacy as vaccine adjuvants. The above cited references within this paragraph are hereby incorporated by reference in their entirety. It is therefore within the purview of the skilled artisan to utilize available adjuvants which may increase the immune response of the polynucleotide vaccines of the present invention in comparison to administration of a non-adjuvanted polynucleotide vaccine.

The DNA vector vaccines of the present invention are administered to the host by any means known in the art, such as enteral and parenteral routes. These routes of delivery include but are not limited to intramusclar injection, intraperitoneal injection, intravenous injection, inhalation or intranasal delivery, oral delivery, sublingual administration, subcutaneous administration, transdermal administration, transcutaneous administration, percutaneous administration or any form of particle bombardment, such as a biolostic device such as a “gene gun” or by any available needle-free injection device. The preferred methods of delivery of the HIV-1 Pol-based DNA vaccines disclosed herein are intramuscular injection, subcutaneous administration and needle-free injection. An especially preferred method is intramuscular delivery.

The amount of expressible DNA to be introduced to a vaccine recipient will depend on the strength of the transcriptional and translational promoters used in the DNA construct, and on the immunogenicity of the expressed gene product. In general, an immunologically or prophylactically effective dose of about 1 μg to greater than about 20 mg, and preferably in doses from about 1 mg to about 5 mg is administered directly into muscle tissue. As noted above, subcutaneous injection, intradermal introduction, impression through the skin, and other modes of administration such as intraperitoneal, intravenous, inhalation and oral delivery are also contemplated. It is also contemplated that booster vaccinations are to be provided in a fashion which optimizes the overall immune response to the Pol-based DNA vector vaccines of the present invention.

The aforementioned polynucleotides, when directly introduced into a vertebrate in vivo, express the respective HIV-1 Pol protein within the animal and in turn induce a cellular immune response within the host to the expressed Pol antigen. To this end, the present invention also relates to methods of using the HIV-1 Pol-based polynucleotide vaccines of the present invention to provide effective immunoprophylaxis, to prevent establishment of an HIV-1 infection following exposure to this virus, or as a post-HIV infection therapeutic vaccine to mitigate the acute HIV-1 infection so as to result in the establishment of a lower virus load with beneficial long term consequences. As noted above, the present invention contemplates a method of administration or use of the DNA pol-based vaccines of the present invention using an any of the known routes of introducing polynucleotides into living tissue to induce expression of proteins.

Therefore, the present invention provides for methods of using a DNA pol-based vaccine utilizing the various parameters disclosed herein as well as any additional parameters known in the art, which, upon introduction into mammalian tissue induces intracellular expression of these DNA pol-based vaccines. This intracellular expression of the Pol-based immunogen induces a cellular immune response which provides a substantial level of protection against an existing HIV-1 infection or provides a substantial level of protection against a future infection in a presently uninfected host.

The following examples are provided to illustrate the present invention without, however, limiting the same hereto.

### EXAMPLE 1

Vaccine Vectors

V1—Vaccine vector V1 was constructed from pCMVIE-AKI-DHFR (Whang et al., 1987, J. Virol. 61: 1796). The AKI and DHFR genes were removed by cutting the vector with EcoRI and self-ligating. This vector does not contain intron A in the CMV promoter, so it was added as a PCR fragment that had a deleted internal SacI site [at 1855 as numbered in Chapman, et al., 1991, Nuc. Acids Res. 19: 3979). The template used for the PCR reactions was pCMVintA-Lux, made by ligating the HindIII and NheI fragment from pCMV6a120 (see Chapman et al., ibid.), which includes hCMV-IE1 enhancer/promoter and intron A, into the HindIII and XbaI sites of pBL3 to generate pCMVIntBL. The 1881 base pair luciferase gene fragment (HindIII-SmaI Klenow filled-in) from RSV-Lux (de Wet et al., 1987, Mol. Cell Biol. 7: 725) was ligated into the SalI site of pCMVIntBL, which was Klenow filled-in and phosphatase treated. The primers that spanned intron A are: 5′ primer: 5′-CTATAT AAGCAGAGCTCGTTTAG-3′ (SEQ ID NO: 10); 3′ primer: 5′-GTAGCAAA GATCTAAGGACGGTGACTGCAG-3′ (SEQ ID NO: 11). The primers used to remove the SacI site are: sense primer, 5′-GTATGTGTCTGAAAATGAGCG TGGAGATTGGGCTCGCAC-3′ (SEQ ID NO: 12) and the antisense primer, 5′-GTGCGAGCCCAATCTCCACGCTCACTTCAGAC ACATAC-3′ (SEQ ID NO: 13). The PCR fragment was cut with Sac I and Bgl II and inserted into the vector which had been cut with the same enzymes.

V1J—Vaccine vector V1J was generated to remove the promoter and transcription termination elements from vector V1 in order to place them within a more defined context, create a more compact vector, and to improve plasmid purification yields. V1J is derived from vectors V1 and pUC18, a commercially available plasmid. V1 was digested with SspI and EcoRI restriction enzymes producing two fragments of DNA. The smaller of these fragments, containing the CMVintA promoter and Bovine Growth Hormone (BGH) transcription termination elements which control the expression of heterologous genes, was purified from an agarose electrophoresis gel. The ends of this DNA fragment were then “blunted” using the T4 DNA polymerase enzyme in order to facilitate its ligation to another “blunt-ended” DNA fragment. pUC18 was chosen to provide the “backbone” of the expression vector. It is known to produce high yields of plasmid, is well-characterized by sequence and function, and is of small size. The entire lac operon was removed from this vector by partial digestion with the HaeII restriction enzyme. The remaining plasmid was purified from an agarose electrophoresis gel, blunt-ended with the T4 DNA polymerase treated with calf intestinal alkaline phosphatase, and ligated to the CMVintA/BGH element described above. Plasmids exhibiting either of two possible orientations of the promoter elements within the pUC backbone were obtained. One of these plasmids gave much higher yields of DNA in E. coli and was designated V1J. This vector's structure was verified by sequence analysis of the junction regions and was subsequently demonstrated to give comparable or higher expression of heterologous genes compared with V1. The nucleotide sequence of V1J is as follows:

| | |
|---|---|
| TCGCGCGTTT CGGTGATGAC GGTGAAAACC TCTGACACAT GCAGCTCCCG GAGACGGTCA | (SEQ ID NO:14) |
| CAGCTTGTCT GTAAGCGGAT GCCGGGAGCA GACAAGCCCG TCAGGGCGCG TCAGCGGGTG | |
| TTGCCGGGTG TCGGGGCTGG CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGQCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TCACCGCCCA ACGACCCCCG CCCATTGACG TCAATAATGA CGTATGTTCC | |
| CATAGTAACG CCAATAGGGA CTTTCCATTG ACGTCAATQG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATCGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACGGGGATTT CCAAGTCTCC ACCCCATTGA | |
| CGTCAATGGG AGTTTGTTTT GGCACCAAAA TCAACGGGAC TTTCCAAAAT GTCGTAACAA | |
| CTCCGCCCCA TTGACGCAAA TGGGCGGTAG GCGTGTACGG TGGGAGGTCT ATATAAGCAG | |
| AGCTCGTTTA GTGAACCGTC AGATCGCCTG GAGACGCCAT CCACGCTGTT TTQACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGAGTC TATAGGCCCA CCCCCTTGGC | |
| TTCTTATGCA TGCTATACTG TTTTTGGCTT GGGGTCTATA CACCCCCGCT TCCTCATGTT | |
| ATAGGTGATG GTATAGCTTA GCCTATAGGT GTGGGTTATT GACCATTATT GACCACTCCC | |
| CTATTGGTGA CGATACTTTC CATTACTAAT CCATAACATG GCTCTTTGCC ACAACTCTCT | |
| TTATTGGCTA TATGCCAATA CACTGTCCTT CAGAGACTGA CACGGACTCT GTATTTTTAC | |
| AGGATGGGGT CTCATTTATT ATTTACA~AT TCACATATAC AACACCACCG TCCCCAGTGC | |
| CCGCAGTTTT TATTAAACAT AACGTGGGAT CTCCACGCGA ATCTCGGGTA CGTGTTCCGG | |
| ACATGGGCTC TTCTCCGGTA GCGGCGGAGC TTCTACATCC GAGCCCTGCT CCCATGCCTC | |
| CAGCGACTCA TGGTCGCTCG GCAGCTCCTT GCTCCTAACA GTGGAGGCCA GACTTAGGCA | |
| CACCAGGATG CCCACCACCA CCAGTGTGCC GCACAAGGCC GTGGCGGTAG GGTATGTGTC | |
| TGAAAATGAG CTCGGGGAGC GGGCTTGCAC CGCTGACGCA TTTGGAAGAC TTAAGGCAGC | |
| GGCAGAAGAA GATGCAGGCA GCTGAGTTGT TGTGTTCTGA TAAGAGTCAG AGGTAACTCC | |
| CGTTGCGGTG CTGTTAACGG TGGAGGGCAG TGTAGTCTGA GCAGTACTCG TTGCTGCCGC | |
| GCGCGCCACC AGACATAATA GCTGACAGAC TAACAGACTG TTCCTTTCCA TGGGTCTTTT | |
| CTGCAGTCAC CGTCCTTAGA TCTGCTGTGC CTTCTAGTTG CCAGCCATCT GTTGTTTGCC | |
| CCTCCCCCGT GCCTTCCTTG ACCCTGGAAG GTGCCACTCC CACTGTCCTT TCCTAATAAA | |
| ATGAGGAAAT TGCATCGCAT TGTCTGAGTA GGTGTCATTC TATTCTGGGG GGTGGGGTGG | |
| GGCAGCACAG CAAGGGGGAG GATTGGGAAG ACAATAGCAG GCATGCTGGG GATGCGGTGG | |
| GCTCTATGGG TACCCAGGTG CTGAAGAATT GACCCGGTTC CTCCTGGGCC AGAAAGAAGC | |
| AGGCACATCC CCTTCTCTGT GACACACCCT GTCCACGCCC CTGGTTCTTA GTTCCAGCCC | |
| CACTCATAGG ACACTCATAG CTCAGGAGGG CTCCGCCTTC AATCCCACCC GCTAAAGTAC | |
| TTGGAGCGGT CTCTCCCTCC CTCATCAGCC CACCAAACCA AACCTAGCCT CCAAGAGTGG | |
| GAAGAAATTA AAGCAAGATA GGCTATTAAG TGCAGAGGGA GAGAAAATGC CTCCAACATG | |
| TGAGGAAGTA ATGAGAGAAA TCATAGAATT TCTTCCGCTT CCTCGCTCAC TGACTCGCTG | |
| CGCTCGGTCG TTCGGCTGCG GCGAGCGGTA TCAGCTCACT CAAAGGCGGT AATACGGTTA | |
| TCCACAGAAT CAGGGGATAA CGCAGGAAAG AACATGTGAG CAAAAGGCCA GCAAAAGGCC | |
| AGGAACCGTA AAAAGGCCGC GTTGCTGGCG TTTTTCCATA GGCTCCGCCC CCCTGACGAG | |
| CATCACAAAA ATCGACGCTC AAGTCAGAGG TGGCGAAACC CGACAGGACT ATAAAGATAC | |
| CAGGCGTTTC CCCCTGGAAG CTCCCTCGTG CGCTCTCCTG TTCCGACCCT GCCGCTTACC | |
| GGATACCTGT CCGCCTTTCT CCCTTCGGGA AGCGTGGCGC TTTCTCAATG CTCACGCTGT | |
| AGGTATCTCA GTTCGGTGTA GGTCGTTCGC TCCAAGCTGG GCTGTGTGCA CGAACCCCCC | |
| GTTCAGCCCG ACCGCTGCGC CTTATCCGGT AACTATCGTC TTGAGTCCAA CCCGGTAAGA | |
| CACGACTTAT CGCCACTGGC AGCAGCCACT GGTAACAGGA TTAGCAGAGC GAGGTATGTA | |
| GGCGGTGCTA CAGAGTTCTT GAAGTGGTGG CCTAACTACG GGTACACTAG AAGGACAGTA | |
| TTTGGTATCT GCGCTCTGCT GAAGCCAGTT ACCTTCGGAA AAAGAGTTGG TAGCTCTTGA | |
| TCCGGCAAAC AAACCACCGC TGGTAGCGGT GGTTTTTTTG TTTGCAAGCA GCAGATTACG | |
| CGCAGAAAAA AAGGATCTCA AGAAGATCCT TTGATCTTTT CTACGGGGTC TGACGCTCAG | |
| TGGAACGAAA ACTCACGTTA AGGGATTTTG GTCATGAGAT TATCAAAAAG GATCTTCACC | |
| TAGATCCTTT TAAATTAAAA ATGAAGTTTT AAATCAATCT AAAGTATATA TGAGTAAACT | |
| TGGTCTGACA GTTACCAATG CTTAATCAGT GAGGCACCTA TCTCAGCGAT CTGTCTATTT | |
| CGTTCATCCA TAGTTGCCTG ACTCCCCGTC GTGTAGATAA CTACGATACG GGAGGGCTTA | |
| CCATCTCGCC CCAGTGCTGC AATGATACCG CGAGACCCAC GCTCACCGGC TCCAGATTTA | |
| TCAGCAATAA ACCAGCCAGC CGGAAGGGCC GAGCGCAGAA GTGGTCCTGC AACTTTATCC | |
| GCCTCCATCC AGTCTATTAA TTGTTGCCGG GAAGCTAGAG TAAGTAGTTC GCCAGTTAAT | |
| AGTTTGCGCA ACGTTGTTGC CATTGCTACA GGCATCGTGG TGTCACGCTC GTCGTTTGGT | |
| ATGGCTTCAT TCAGCTCCGG TTCCCAACGA TCAAGGCGAG TTACATGATC CCCCATGTTG | |
| TGCAAAAAAG CGGTTAGCTC CTTCGGTCCT CCGATCGTTG TCAGAAGTAA GTTGGCCGCA | |
| GTGTTATCAC TCATGGTTAT GGCAGCACTG CATAATTCTC TTACTGTCAT GCCATCCGTA | |
| AGATGCTTTT CTGTGACTGG TGAGTACTCA ACCAAGTCAT TCTGAGAATA GTGTATGCGG | |
| CGACCGAGTT GCTCTTGCCC GGCCTCAATA CGGGATAATA CCGCGCCACA TAGCAGAACT | |
| TTAAAAGTGC TCATCATTGG AAAACGTTCT TCGGGGCGAA AACTCTCAAG GATCTTACCG | |
| CTGTTGAGAT CCAGTTCGAT GTAACCCACT CGTGCACCCA ACTGATCTTC AGCATCTTTT | |
| ACTTTCACCA GCGTTTCTGG GTGAGCAAAA ACAGGAAGGC AAAATGCCGC AAAAAAGGGA | |
| ATAAGGGCGA CACGGAAATG TTGAATACTC ATACTCTTCC TTTTTCAATA TTATTGAAGC | |
| ATTTATCAGG GTTATTGTCT CATGAGCGGA TACATATTTG AATGTATTTA GAAAAATAAA | |
| CAAATAGGGG TTCCGCGCAC ATTTCCCCGA AAAGTGCCAC CTGACGTCTA AGAAACCATT | |
| ATTATCATGA CATTAACCTA TAAAAATAGG CGTATCACGA GGCCCTTTCG TC. | |

V1Jneo—Construction of vaccine vector V1Jneo expression vector involved removal of the amp^{r} gene and insertion of the kan^{r} gene (neomycin phosphotransferase). The amp^{r} gene from the pUC backbone of V1J was removed by digestion with SspI and Eam1105I restriction enzymes. The remaining plasmid was purified by agarose gel electrophoresis, blunt-ended with T4 DNA polymerase, and then treated with calf intestinal alkaline phosphatase. The commercially available kan^{r} gene, derived from transposon 903 and contained within the pUC4K plasmid, was excised using the PstI restriction enzyme, purified by agarose gel electrophoresis, and blunt-ended with T4 DNA polymerase. This fragment was ligated with the V1J backbone and plasmids with the kan^{r} gene in either orientation were derived which were designated as V1Jneo #'s 1 and 3. Each of these plasmids was confirmed by restriction enzyme digestion analysis, DNA sequencing of the junction regions, and was shown to produce similar quantities of plasmid as V1J. Expression of heterologous gene products was also comparable to V1J for these V1Jneo vectors. V1Jneo#3, referred to as V1Jneo hereafter, was selected which contains the kan^{r} gene in the same orientation as the amp^{r} gene in V1J as the expression construct and provides resistance to neomycin, kanamycin and G418. The nucleotide sequence of V1Jneo is as follows:

| | |
|---|---|
| TCGCGCGTTT CGGTGATGAC GGTGAAAACC TCTGACACAT GCAGCTCCCG GAGACGGTCA | (SEQ ID NO:15) |
| CAGCTTGTCT GTAAGCGGAT GCCGGGAGCA GACAAGCCCG TCAGGGCGCG TCAGCGGGTG | |
| TTGGCGGGTG TCGGGGCTGG CTTAACTATG CGGCATCAGA GCAGATTGTA CTGAGAGTGC | |
| ACCATATCCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATGG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACGGGGATTT CCAAGTCTCC ACCCCATTGA | |
| CGTCAATGGG AGTTTGTTTT GGCACCAAAA TCAACGGGAC TTTCCAAAAT GTCGTAACAA | |
| CTCCGCCCCA TTGACGCAAA TGGGCGGTAG GCGTGTACGG TGGGAGGTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGAGTC TATAGGCCCA CCCCCTTGGC | |
| TTCTTATGCA TGCTATACTG TTTTTGGCTT GGGGTCTATA CACCCCCGCT TCCTCATGTT | |
| ATAGGTGATG GTATAGCTTA GCCTATACGT GTGGGTTATT GACCATTATT GACCACTCCC | |
| CTATTGGTGA CGATACTTTC CATTACTAAT CCATAACATG GCTCTTTGCC ACAACTCTCT | |
| TTATTGGCTA TATGCCAATA CACTCTCCTT CAGAGACTGA CACGGACTCT GTATTTTTAC | |
| AGGATGGGGT CTCATTTATT ATTTACAAAT TCACATATAC AACACCACCG TCCCCAGTGC | |
| CCGCAGTTTT TATTAAACAT AACGTGGGAT CTCCACGCGA ATCTCGGGTA CGTGTTCCGG | |
| ACATGGGGTC TTCTCCGGTA GCGCCGGAGC TTCTACATCC GAGCCCTGCT CCCATGCCTC | |
| CAGCGACTCA TGGTCGCTCG GCAGCTCCTT GCTCCTAACA GTGGAGGCCA GACTTAGGGA | |
| CAGCACGATG CCCACCACCA CCAGTGTGCC GCACAAGGCC GTGGCGGTAG GGTATGTGTC | |
| TGAAAATGAG CTCGGGGAGC GGGCTTGCAC CGCTGACGCA TTTGGAAGAC TTAAGGCAGC | |
| GGCAGAAGAA GATGCAGGCA GCTGAGTTGT TGTGTTCTGA TAAGAGTCAG AGGTAACTCC | |
| CGTTGCGGTG CTGTTAACGG TGGACGGCAG TGTAGTCTGA GCAGTACTCG TTGCTGCCGC | |
| GCGCGCCACC AGACATAATA GCTGACAGAC TAACAGACTG TTCCTTTCCA TGGGTCTTTT | |
| CTGCAGTCAC CGTCCTTAGA TCTGCTGTGC CTTCTAGTTG CCAGCCATCT GTTGTTTGCC | |
| CCTCCCCCGT GCCTTCCTTG ACCCTGGAAG GTGCCACTCC CACTGTCCTT TGCTAATAAA | |
| ATGAGGAAAT TGCATCGCAT TGTCTGAGTA GGTGTCATTC TATTCTGGGG GGTGGGGTGG | |
| GGCAGCACAG CAAGGGGGAG GATTGGGAAG ACAATAGCAG GCATGCTGGG GATGCGGTGG | |
| GCTCTATGGG TACCCAGGTG CTGAAGAATT GACCCGGTTC CTCCTGGGCC AGAAAGAAGC | |
| AGGCACATCC CCTTCTCTGT GACACACCCT GTCCACGCCC CTGGTTCTTA GTTCCAGCCC | |
| CACTCATAGG ACACTCATAG CTCAGGAGGG CTCCGCCTTC AATCCCACCC GCTAAAGTAC | |
| TTGGAGCGGT CTCTCCCTCC CTCATCAGCC CACCAAACCA AACCTAGCCT CCAAGAGTGG | |
| GAAGAAATTA AAGCAAGATA GGCTATTAAG TGCAGAGGGA GAGAAAATGC CTCCAACATG | |
| TGAGGAAGTA ATGAGAGAAA TCATAGAATT TCTTCCGGTT CCTCGCTCAC TGACTCGCTG | |
| CGCTCGGTCG TTCGGCTGCG GCGAGCGGTA TCAGCTCACT CAAAGGCGGT AATACGGTTA | |
| TCCACAGAAT CAGGGGATAA CGCAGGAAAG AACATGTGAG CAAAAGGCCA GCAAAAGGCC | |
| AGGAACCGTA AAAAGGCCGC GTTGCTGGCG TTTTTCCATA GGCTCCGCCC CCCTGACGAG | |
| CATCACAAAA ATCGACGCTC AAGTCAGAGG TGGCGAAACC CGACAGGACT ATAAAGATAC | |
| CAGGCGTTTC CCCCTGGAAG CTCCCTCGTC CGCTCTCCTG TTCCGACCCT GCCGCTTACC | |
| GGATACCTCT CCGCCTTTCT CCCTTCGGGA AGCGTGGCGC TTTCTCAATG CTCACGCTGT | |
| AGGTATCTCA GTTCGGTGTA GGTCGTTCGC TCCAAGCTGG GCTGTGTGCA CGAACCCCCC | |
| GTTCAGCCCG ACCGCTGCGC CTTATCCGGT AACTATCGTC TTGAGTCCAA CCCGGTAAGA | |
| CACGACTTAT CCCCACTGGC AGCAGCCACT GGTAACAGGA TTAGCAGAGC GAGGTATGTA | |
| GGCGGTGCTA CAGAGTTCTT GAAGTGGTGG CCTAACTACG GCTACACTAG AAGGACAGTA | |
| TTTGGTATCT GCGCTCTCCT GAAGCCAGTT ACCTTCCGAA AAAGAGTTGG TAGCTCTTGA | |
| TCCGGCAAAC AAACCACCGC TGGTAGCGGT GGTTTTTTTG TTTGCAAGCA GCAGATTACG | |
| CGCAGAAAAA AAGGATCTCA AGAAGATCCT TTGATCTTTT CTACGGGGTC TGACGCTCAG | |
| TGGAACGAAA ACTCACGTTA AGGGATTTTG GTCATGAGAT TATCAAAAAG GATCTTCACC | |
| TAGATCCTTT TAAATTAAAA ATGAAGTTTT AAATCAATCT AAAGTATATA TGAGTAAACT | |
| TGGTCTGACA GTTACCAATG CTTAATCAGT GAGGCACCTA TCTCAGCGAT CTGTCTATTT | |
| CGTTCATCCA TAGTTGCCTG ACTCCGGGGG GGGGGGGCGC TGAGGTCTGC CTCGTGAAGA | |
| AGGTGTTGCT GACTCATACC AGGCCTGAAT CGCCCCATCA TCCAGCCAGA AAGTGAGGGA | |
| GCCACGGTTG ATGAGAGCTT TGTTGTAGGT GGACCAGTTG GTGATTTTGA ACTTTTGCTT | |
| TGCCACGGAA CGGTCTCCGT TGTCGGGAAG ATGCGTGATC TGATCCTTCA ACTCAGCAAA | |
| AGTTCGATTT ATTCAACAAA GCCGCCGTCC CGTCAAGTCA GCGTAATGCT CTCCCAGTGT | |
| TACAACCAAT TAACCAATTC TGATTAGAAA AACTCATCGA GCATCAAATG AAACTGCAAT | |
| TTATTCATAT CAGGATTATC AATACCATAT TTTTGAAAAA GCCGTTTCTG TAATGAAGGA | |
| GAAAACTCAC CGAGGCAGTT CCATAGGATG GCAAGATCCT GGTATCGGTC TGCGATTCCG | |
| ACTCGTCCAA CATCAATACA ACCTATTAAT TTCCCCTCGT CAAAAATAAG GTTATCAAGT | |
| GAGAAATCAC CATGAGTGAC GACTGAATCC GGTGAGAATG GCAAAAGCTT ATGCATTTCT | |
| TTCCAGACTT GTTCAACACG CCAGCCATTA CGCTCGTCAT CAAAATCACT CGCATCAACC | |
| AAACCGTTAT TCAWTCGTGA TTGCGCCTGA GCGAGACGAA ATACGCGATC GCTGTTAAAA | |
| GGACAATTAC AAACAGGAAT CGAATGCAAC CGGCGCAGGA ACACTCCCAG CGCATCAACA | |
| ATATTTTCAC CTGAATCAGG ATATTCTTCT AATACCTGGA ATGCTGTTTT CCCGGGGATC | |
| GCAGTGGTGA GTAACCATGC ATCATCAGGA GTACGGATAA AATGCTTGAT GGTCGGAACA | |
| GGCATAAATT CCGTCAGCCA GTTTAGTCTG ACCATCTCAT CTGTAACATC ATTGGCAACG | |
| CTACCTTTGC CATGTTTCAG AAACAACTCT GGCGCATCGG GCTTCCCATA CAATCGATAG | |
| ATTGTCGCAC CTGATTGCCC GACATTATCG CGAGCCCATT TATACCCATA TAAATCAGCA | |
| TCCATGTTGG AATTTAATCG CGGGCTGGAG CAAGACGTTT CCCGTTGAAT ATGGCTCATA | |
| ACACCCCTTG TATTACTGTT TATGTAAGCA GACAGTTTTA TTGTTCATGA TGATATATTT | |
| TTATCTTGTG CAATGTAACA TCAGAGATTT TGAGACACAA CGTGGCTTTC CCCCCCCCCC | |
| CATTATTGAA GCATTTATCA GGGTTATTGT CTCATGAGCG GATACATATT TGAATGTATT | |
| TAGAAAAATA AACAAATAGG GGTTCCGCGC ACATTTCCCC GAAAAGTGCC ACCTGACGTC | |
| TAAGAAACCA TTATTATCAT GACATTAACC TATAAAAATA GGCGTATCAC GAGGCCCTTT | |
| CGTC. | |

| | |
|---|---|
| CCTCCCCCGT GCCTTCCTTG ACCCTGGAAG GTGCCACTCC CACTGTCCTT TGCTAATAAA | (SEQ ID NO:15) |
| ATGAGGAAAT TGCATCGCAT TGTCTGAGTA GGTGTCATTC TATTCTGGGG GGTGGGGTGG | |
| GGCAGCACAG CAAGGGGGAG GATTGGGAAG ACAATAGCAG GCATGCTGGG GATGCGGTGG | |
| GCTCTATGGG TACCCAGGTG CTGAAGAATT GACCCGGTTC CTCCTGGGCC AGAAAGAAGC | |
| AGGCACATCC CCTTCTCTGT GACACACCCT GTCCACGCCC CTGGTTCTTA GTTCCAGCCC | |
| CACTCATAGG ACACTCATAG CTCAGGAGGG CTCCGCCTTC AATCCCACCC GCTAAAGTAC | |
| TTGGAGCGGT CTCTCCCTCC CTCATCAGCC CACCAAACCA AACCTAGCCT CCAAGAGTGG | |
| GAAGAAATTA AAGCAAGATA GGCTATTAAG TGCAGAGGGA GAGAAAATGC CTCCAACATG | |
| TGAGGAAGTA ATGAGAGAAA TCATAGAATT TCTTCCGGTT CCTCGCTCAC TGACTCGCTG | |
| CGCTCGGTCG TTCGGCTGCG GCGAGCGGTA TCAGCTCACT CAAAGGCGGT AATACGGTTA | |
| TCCACAGAAT CAGGGGATAA CGCAGGAAAG AACATGTGAG CAAAAGGCCA GCAAAAGGCC | |
| AGGAACCGTA AAAAGGCCGC GTTGCTGGCG TTTTTCCATA GGCTCCGCCC CCCTGACGAG | |
| CATCACAAAA ATCGACGCTC AAGTCAGAGG TGGCGAAACC CGACAGGACT ATAAAGATAC | |
| CAGGCGTTTC CCCCTGGAAG CTCCCTCGTC CGCTCTCCTG TTCCGACCCT GCCGCTTACC | |
| GGATACCTCT CCGCCTTTCT CCCTTCGGGA AGCGTGGCGC TTTCTCAATG CTCACGCTGT | |
| AGGTATCTCA GTTCGGTGTA GGTCGTTCGC TCCAAGCTGG GCTGTGTGCA CGAACCCCCC | |
| GTTCAGCCCG ACCGCTGCGC CTTATCCGGT AACTATCGTC TTGAGTCCAA CCCGGTAAGA | |
| CACGACTTAT CCCCACTGGC AGCAGCCACT GGTAACAGGA TTAGCAGAGC GAGGTATGTA | |
| GGCGGTGCTA CAGAGTTCTT GAAGTGGTGG CCTAACTACG GCTACACTAG AAGGACAGTA | |
| TTTGGTATCT GCGCTCTCCT GAAGCCAGTT ACCTTCCGAA AAAGAGTTGG TAGCTCTTGA | |
| TCCGGCAAAC AAACCACCGC TGGTAGCGGT GGTTTTTTTG TTTGCAAGCA GCAGATTACG | |
| CGCAGAAAAA AAGGATCTCA AGAAGATCCT TTGATCTTTT CTACGGGGTC TGACGCTCAG | |
| TGGAACGAAA ACTCACGTTA AGGGATTTTG GTCATGAGAT TATCAAAAAG GATCTTCACC | |
| TAGATCCTTT TAAATTAAAA ATGAAGTTTT AAATCAATCT AAAGTATATA TGAGTAAACT | |
| TGGTCTGACA GTTACCAATG CTTAATCAGT GAGGCACCTA TCTCAGCGAT CTGTCTATTT | |
| CGTTCATCCA TAGTTGCCTG ACTCCGGGGG GGGGGGGCGC TGAGGTCTGC CTCGTGAAGA | |
| AGGTGTTGCT GACTCATACC AGGCCTGAAT CGCCCCATCA TCCAGCCAGA AAGTGAQGGA | |
| GCCACGGTTG ATGAGAGCTT TGTTGTAGGT GGACCAGTTG GTGATTTTGA ACTTTTGCTT | |
| TGCCACGGAA CGGTCTCCGT TGTCGGGAAG ATGCGTGATC TGATCCTTCA ACTCAGCAAA | |
| AGTTCGATTT ATTCAACAAA GCCGCCGTCC CGTCAAGTCA GCGTAATGCT CTCCCAGTGT | |
| TACAACCAAT TAACCAATTC TGATTAGAAA AACTCATCGA GCATCAAATG AAACTGCAAT | |
| TTATTCATAT CAGGATTATC AATACCATAT TTTTGAAAAA GCCGTTTCTG TAATGAAGGA | |
| GAAAACTCAC CGAGGCAGTT CCATAGGATG GCAAGATCCT GGTATCGGTC TGCGATTCCG | |
| ACTCGTCCAA CATCAATACA ACCTATTAAT TTCCCCTCGT CAAAAATAAG GTTATCAAGT | |
| GAGAAATCAC CATGAGTGAC GACTGAATCC GGTGAGAATG GCAAAAGCTT ATGCATTTCT | |
| TTCCAGACTT GTTCAACACG CCAGCCATTA CGCTCGTCAT CAAAATCACT CGCATCAACC | |
| AAACCGTTAT TCAWrCGTGA TTGCGCCTGA GCGAGACGAA ATACGCGATC GCTGTTAAAA | |
| GGACAATTAC AAACAGGAAT CGAATGCAAC CGGCGCAGGA ACACTCCCAG CGCATCAACA | |
| ATATTTTCAC CTGAATCAGG ATATTCTTCT AATACCTGGA ATGCTGTTTT CCCGGGGATC | |
| GCAGTGGTGA GTAACCATGC ATCATCAGGA GTACGGATAA AATGCTTGAT GGTCGGAACA | |
| GGCATAAATT CCGTCAGCCA GTTTAGTCTG ACCATCTCAT CTGTAACATC ATTGGCAACG | |
| CTACCTTTGC CATGTTTCAG AAACAACTCT GGCGCATCGG GCTTCCCATA CAATCGATAG | |
| ATTGTCGCAC CTGATTGCCC QACATTATCG CGAGCCCATT TATACCCATA TAAATCAGCA | |
| TCCATGTTGG AATTTAATCG CGGGCTGGAG CAAGACGTTT CCCGTTGAAT ATGGCTCATA | |
| ACACCCCTTG TATTACTGTT TATGTAAGCA GACAGTTTTA TTGTTCATGA TGATATATTT | |
| TTATCTTGTG CAATGTAACA TCAGAGATTT TGAGACACAA CGTGGCTTTC CCCCCCCCCC | |
| CATTATTGAA GCATTTATCA GGGTTATTGT CTCATGAGCG GATACATATT TGAATGTATT | |
| TAGAAAAATA AACAAATAGG GGTTCCGCGC ACATTTCCCC GAAAAGTGCC ACCTGACGTC | |
| TAAGAAACCA TTATTATCAT GACATTAACC TATAAAAATA GGCGTATCAC GAGGCCCTTT | |
| CGTC. | |

V1Jns—The expression vector VIJns was generated by adding an SfiI site to V1Jneo to facilitate integration studies. A commercially available 13 base pair SfiI linker (New England BioLabs) was added at the KpnI site within the BGH sequence of the vector. V1Jneo was linearized with KpnI, gel purified, blunted by T4 DNA polymerase, and ligated to the blunt SfiI linker. Clonal isolates were chosen by restriction mapping and verified by sequencing through the linker. The new vector was designated V1Jns. Expression of heterologous genes in V1Jns (with SfiI) was comparable to expression of the same genes in V1Jneo (with KpnI).

The nucleotide sequence of V1Jns is as follows:

| | |
|---|---|
| TCGCGCGTTT CGGTGATGAC GGTGAAAACC TCTGACACAT GCAGCTCCCG GAGACGGTCA | (SEQ ID NO:16) |
| CAGCTTGTCT GTAAGCGGAT GCCGGGAGCA GACAAGCCCG TCAGGGCGCG TCAGCGGGTG | |
| TTGGCGGGTG TCGGGGCTGG CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATGG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACCGGGATTT CCAAGTCTCC ACCCCATTGA | |
| CGTCAATGGG AGTTTGTTTT CGCACCAAAA TCAACGGGAC TTTCCAAAAT GTCGTAACAA | |
| CTCCGCCCCA TTGACGCAAA TGGGCGGTAG GCGTGTACGG TGGGAGGTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGACTC TATAGGCACA CCCCTTTGGC | |
| TCTTATGCAT GCTATACTGT TTTTGGCTTG GGGCCTATAC ACCCCCGCTT CCTTATGCTA | |
| TAGGTGATGG TATAGCTTAG CCTATAGGTG TGGGTTATTG ACCATTATTG ACCACTCCCC | |
| TATTGGTGAC GATACTTTCC ATTACTAATC CATAACATGG CTCTTTGCCA CAACTATCTC | |
| TATTGGCTAT ATGCCAATAC TCTGTCCTTC AGAGACTGAC ACGGACTCTG TATTTTTACA | |
| GGATGCGGTC CCATTTATTA TTTACAAATT CACATATACA ACAACGCCGT CCCCCGTGCC | |
| CGCAGTTTTT ATTAAACATA GCGTGCGATC TCCACGCGAA TCTCGGGTAC GTGTTCCGGA | |
| CATGGGCTCT TCTCCGGTAG CGGCGCAGCT TCCACATCCG AGCCCTGGTC CCATGCCTCC | |
| AGCGGCTCAT GGTCGCTCGG CAGCTCCTTG CTCCTAACAG TGGAGGCCAG ACTTAGGCAC | |
| AGCACAATGC CCACCACCAC CAGTGTGCCG CACAAGGCCG TGGCGGTAGG GTATGTGTCT | |
| GAAAATGAGC GTGGAGATTG GGCTCGCACG GCTGACGCAG ATGGAAGACT TAAGGCAGCG | |
| GCAGAAGAAG ATGCAGGCAG CTGAGTTGTT GTATTCTGAT AAGAGTCAGA GGTAACTCCC | |
| GTTGCGGTGC TGTTAACGGT GGAGGGCAGT GTAGTCTGAG CAGTACTCGT TGCTGCCGCG | |
| CGCGCCACCA GACATAATAG CTGACAGACT AACAGACTGT TCCTTTCCAT GGGTCTTTTC | |
| TGCAGTCACC GTCCTTAGAT CTGCTGTGCC TTCTAGTTGC CAGCCATCTG TTGTTTGCCC | |
| CTCCCCCGTG CCTTCCTTGA CCCTGGAAGG TGCCACTCCC ACTGTCCTTT CCTAATAAAA | |
| TGAGGAAATT GCATCGCATT GTCTGAGTAG GTGTCATTCT ATTCTGGGGG GTGGGGTGGG | |
| GCAGGACAGC AAGGGGGAGG ATTGGGAAGA CAATAGCAGG CATGCTQGGG ATGCGGTGGG | |
| CTCTATGGCC GCTCCGGCCA GGTGCTGAAG AATTGACCCG GTTCCTCCTG GGCCAGAAAG | |
| AAGCAGCCAC ATCCCCTTCT CTGTGACACA CCCTGTCCAC GCCCCTGGTT CTTAGTTCCA | |
| GCCCCACTCA TAGGACACTC ATAGCTCAGG AGGGCTCCGC CTTCAATCCC ACCCGCTAAA | |
| GTACTTGGAG CGGTCTCTCC CTCCCTCATC AGCCCACCAA ACCAAACCTA GCCTCCAAGA | |
| GTGGGAAGAA ATTAAAGCAA GATAGGCTAT TAAGTGCAGA GGGAGAGAAA ATGCCTCCAA | |
| CATGTGAGGA AGTAATGAGA GAAATCATAG AATTTCTTCC GCTTCCTCGC TGACTGACTG | |
| GCTGCGCTCG GTCGTTCCGC TGCGGCGAGC GGTATCAGCT CACTCAAAGG CGGTAATACG | |
| GTTATCCACA GAATCAGGGG ATAACGCAGG AAAGAACATG TGAGCAAAAG GCCAGCAAAA | |
| GGCCAGGAAC CGTAAAAAGG CCGCGTTGCT GGCGTTTTTC CATAGGCTCC GCCCCCCTGA | |
| CGAGCATCAC AAAAATCGAC GCTCAAGTCA GAGGTGGCGA AACCCGACAG GACTATAAAG | |
| ATACCAGGGG TTTCCCCCTG GAAGCTCCCT CGTGCGCTCT CCTGTTCCGA CCCTGCCGCT | |
| TACCGGATAC CTGTCCGCCT TTCTCCCTTC GGGAAGCGTG GCGCTTTCTC ATAGCTCACG | |
| CTGTAGGTAT CTCAGTTCGG TGTAGGTCGT TCGCTCCAAG CTGGGCTGTG TGCACGAACC | |
| CCCCGTTCAG CCCGACCGCT GCGCCTTATC CGGTAACTAT CGTCTTGAGT CCAACCCGGT | |
| AAGACACGAC TTATCGCCAC TGGCAGCAGC CACTGGTAAC AGGATTACCA GAGCGAGGTA | |
| TGTAGGCGGT GCTACAGAGT TCTTGAAGTG GTGGCCTAAC TACGGCTACA CTAGAAGAAC | |
| AGTATTTGGT ATCTGCGCTC TGCTGAAGCC AGTTACCTTC GGAAAAAGAG TTGGTAGCTC | |
| TTGATCCGGC AAACAAACCA CCGCTGGTAG CGGTGGTTTT TTTGTTTGCA AGCAGCAGAT | |
| TACGCGCAGA AAAAAAGGAT CTCAAGAAGA TCCTTTGATC TTTTCTACGG GGTCTGACGC | |
| TCAGTGGAAC GAAAACTCAC GTTAAGGGAT TTTGGTCATG AGATTATCAA AAAGGATCTT | |
| CACGTAGATG CTTTTAAATT AAAAATGAAG TTTTAAATCA ATCTAAAGTA TATATGAGTA | |
| AACTTGGTCT GACAGTTACC AATGCTTAAT CAGTGAGGCA CCTATCTCAG CGATCTGTCT | |
| ATTTCGTTCA TCCATAGTTG CCTGACTCGG GGGGGGGGGG CGCTGAGGTC TGCCTCGTGA | |
| AGAAGGTGTT GCTGACTCAT ACCAGGCCTG AATCGCCCCA TCATCCAGCC AGAAAGTGAG | |
| GGAGCCACGG TTGATGAGAG CTTTGTTGTA GGTGGACCAG TTGGTGATTT TGAACTTTTG | |
| CTTTGCCACG GAACGGTCTG CGTTGTCGGG AAGATGCGTG ATCTGATCCT TCAACTCAGC | |
| AAAAGTTCGA TTTATTCAAC AAAGCCGCCG TCCCGTCAAG TCAGCGTAAT GCTCTGCCAG | |
| TGTTACAACC AATTAACCAA TTCTGATTAG AAAAACTCAT CGAGCATCAA ATGAAACTGC | |
| AATTTATTCA TATCAGGATT ATCAATACCA TATTTTTGAA AAAGCCGTTT CTGTAATGAA | |
| GGAGAAAACT CACCGAGGCA GTTCCATAGG ATGGCAAGAT CCTGGTATCG GTCTGCGATT | |
| CCGACTCGTC CAACATCAAT ACAACCTATT AATTTCCCCT CGTCAAAAAT AAGGTTATCA | |
| AGTGAGAAAT CACCATGAGT GACGACTGAA TCCGGTGAGA ATGGCAAAAG CTTATGCATT | |
| TCTTTCCAGA CTTGTTCAAC AGGGCAGGCA TTACGCTCGT CATCAAAATC ACTCGCATCA | |
| ACCAAACCGT TATTCATTCG TGATTGCGCC TGAGCGAGAC GAAATACGCG ATCGCTGTTA | |
| AAAGGACAAT TACAAACAGG AATCGAATGC AACCGGCGCA GGAACACTGC CAGCGCATCA | |
| ACAATATTTT CACCTGAATC AGGATATTCT TCTAATACCT GGAATGCTGT TTTCCCGGGG | |
| ATCGCAGTGG TGAGTAACCA TGCATCATCA GGAGTACGGA TAAAATGCTT GATGGTCGGA | |
| AGAGGCATAA ATTCCGTCAG CCAGTTTAGT CTGACCATCT CATCTGTAAC ATCATTGGCA | |
| ACGCTACCTT TGCCATGTTT CAGAAACAAC TCTGGCGCAT CGGGCTTCCC ATACAATCGA | |
| TAGATTGTCG CACCTGATTG CCCGACATTA TCGCGAGCCC ATTTATACCC ATATAAATCA | |
| GCATCCATGT TGGAATTTAA TCGCGGCCTC GAGCAAGACG TTTCCCGTTG AATATGGCTC | |
| ATAACACCCC TTGTATTACT GTTTATGTAA GCAGACAGTT TTATTGTTCA TGATGATATA | |
| TTTTTATCTT GTGCAATGTA ACATCAGAGA TTTTGAGACA CAACGTGGCT TTCCCCCCCC | |
| CCCGATTATT GAAGCATTTA TCAGGGTTAT TGTCTCATGA GCGGATACAT ATTTGAATGT | |
| ATTTAGAAAA ATAAACAAAT AGGGGTTCCG CGCACATTTC CCCGAAAAGT GCCACCTGAC | |
| GTCTAAGAAA GCATTATTAT CATGACATTA ACCTATAAAA ATAGGCGTAT CACGAGGCCC | |
| TTTCGTC. | |

The underlined nucleotides of SEQ ID NO: 16 represent the Sfi1 site introduced into the Kpn 1 site of V1Jneo.

V1Jns-tPA—The vaccine vector V1Jns-tPA was constructed in order to fuse an heterologous leader peptide sequence to the pol DNA constructs of the present invention. More specifically, the vaccine vector V1Jns was modified to include the human tissue-specific plasminogen activator (tPA) leader. As an exemplification, but by no means a limitation of generating a pol DNA construct comprising an amino-terminal leader sequence, plasmid V1Jneo was modified to include the human tissue-specific plasminogen activator (tPA) leader. Two synthetic complementary oligomers were annealed and then ligated into V1Jneo which had been BglII digested. The sense and antisense oligomers were 5′-GATCACCATGGATGCAATGAAGAG AGGGCTCTGCTGTGTGCTGCTGCTGTGTGGAGCAGTCTTCGTTTCGCCCAG CGA-3′ (SEQ ID NO: 17); and, 5′-GATCTCGCTGGGCGAAACGAAGACTGCTCC ACACAGCAGCAGCACACAGCAGAGCCCTCTCTTICATTGCATCCATGGT-3′ (SEQ ID NO: 18). The Kozak sequence is underlined in the sense oligomer. These oligomers have overhanging bases compatible for ligation to BglII-cleaved sequences. After ligation the upstream BglII site is destroyed while the downstream BglII is retained for subsequent ligations. Both the junction sites as well as the entire tPA leader sequence were verified by DNA sequencing. Additionally, in order to conform with V1Jns (=V1Jneo with an SfiI site), an SfiI restriction site was placed at the KpnI site within the BGH terminator region of V1Jneo-tPA by blunting the KpnI site with T4 DNA polymerase followed by ligation with an SfiI linker (catalogue #1138, New England Biolabs), resulting in V1Jns-tPA. This modification was verified by restriction digestion and agarose gel electrophoresis.

The V1Jns-tpa vector nucleotide sequence is as follows:

| | |
|---|---|
| TCGCGCGTTT CGGTGATGAC GGTGAAAACC TCTGACACAT GCAGCTCCCG GAGACGGTCA | (SEQ ID NO:9) |
| CAGCTTGTCT GTAAGCGGAT GCCGGGAGCA GACAAGCCCG TCAGGGCGCG TCAGCGGGTG | |
| TTGGCGGGTG TCGGGGCTGG CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATGG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACGGGGATTT CCAAGTCTCC ACCCCATTGA | |
| CGTCAATGGG AGTTTGTTTT GGCACCAAAA TCAACGGGAC TTTCCAAAAT GTCGTAACAA | |
| CTCCGCCCCA TTGACGCAAA TGGGCGGTAG GCGTGTACGG TGGGAGGTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGACTC TATAGGCACA CCCCTTTGGC | |
| TCTTATGCAT GCTATACTGT TTTTGGCTTG GGGCCTATAC ACCCCCGCTT CCTTATGCTA | |
| TAGGTGATGG TATAGCTTAG CCTATAGGTG TGGGTTATTG ACCATTATTG ACCACTCCCC | |
| TATTGGTGAC GATACTTTCC ATTACTAATC CATAACATGG CTCTTTGCCA CAACTATCTC | |
| TATTGGCTAT ATGCCAATAC TCTGTCCTTC AGAGACTGAC ACGGACTCTG TATTTTTACA | |
| GGATGGGGTC CCATTTATTA TTTACAAATT CACATATACA ACAACGCCGT CCCCCGTGCC | |
| CGCAGTTTTT ATTAAACATA GCGTGGGATC TCCACGCGAA TCTCGGGTAC GTGTTCCGGA | |
| CATGGGCTCT TCTCCGGTAG CGGCGGAGCT TCCACATCCG AGCCCTGGTC CCATGCCTCC | |
| AGCGGCTCAT GGTCGCTCGG CAGCTCCTTG CTCCTAACAG TGGAGGCCAG ACTTAGGCAC | |
| AGCACAATGC CCACCACCAC CAGTGTGCCG CACAAGGCCG TGGCGGTAGG GTATGTGTCT | |
| GAAAATGAGC GTGGAGATTG GGCTCGCACG GCTGACGCAG ATGGAAGACT TAAGGCAGCG | |
| GCAGAAGAAG ATGCAGGCAG CTGAGTTGTT GTATTCTGAT AAGAGTCAGA GGTAACTCCC | |
| GTTGCGGTGC TGTTAACGGT GGAGGGCAGT GTAGTCTGAG CAGTACTCGT TGCTGCCGCG | |
| CGCGCCACCA GACATAATAG CTGACAGACT AACAGACTGT TCCTTTCCAT GGGTCTTTTC | |
| TGCAGTCACC GTCCTTAGAT CACCATGGAT GCAATGAAGA GAGGGCTCTG CTGTGTGCTG | |
| CTGCTGTGTG GAGCAGTCTT CGTTTCGCCC AGCGAGATCT GCTGTGCCTT CTAGTTGCCA | |
| GCCATCTGTT GTTTGCCCCT CCCCCGTGCC TTCCTTGACC CTGGAAGGTG CCACTCCCAC | |
| TGTCCTTTCC TAATAAAATG AGGAAATTGC ATCGCATTGT CTGAGTAGGT GTCATTCTAT | |
| TCTGGGGGGT GGGGTGGGGC AGGACAGCAA GGGGGAGGAT TGGGAAGACA ATAGCAGGCA | |
| TGCTGGGGAT GCGGTGGGCT CTATGGCCGC TGCGGCCAGG TGCTGAAGAA TTGACCCGGT | |
| TCCTCCTGGG CCAGAAAGAA GCAGGCACAT CCCCTTCTCT GTGACACACC CTGTCCACGC | |
| CCCTGGTTCT TAGTTCCAGC CCCACTCATA GGACACTCAT AGCTCAGGAG GGCTCCGCCT | |
| TCAATCCCAC CCGCTAAAGT ACTTGGAGCG GTCTCTCCCT CCCTCATCAG CCCACCAAAC | |
| CAAACCTAGC CTCCAAGAGT GGGAAGAAAT TAAAGCAAGA TAGGCTATTA AGTGCAGAGG | |
| GAGAGAAAAT GCCTCCAACA TGTGAGGAAG TAATGAGAGA AATCATAGAA TTTCTTCCGC | |
| TTCCTCGCTC ACTGACTCGC TGCGCTCGGT CGTTCGGCTG CGGCGAGCGG TATCAGCTCA | |
| CTCAAAGGCG GTAATACGGT TATCCACAGA ATCAGGGGAT AACGCAGGAA AGAACATGTG | |
| AGCAAAAGGC CAGCAAAAGG CCAGGAACCG TAAAAAGGCC GCGTTGCTGG CGTTTTTCCA | |
| TAGGCTCCGC CCCCCTGACG AGCATCACAA AAATCGACGC TCAAGTCAGA GGTGGCGAAA | |
| CCCGACAGGA CTATAAAGAT ACCAGGCGTT TCCCCCTGGA AGCTCCCTCG TGCGCTCTCC | |
| TGTTCCGACC CTGCCGCTTA CCGGATACCT GTCCGCCTTT CTCCCTTCGG GAAGCGTGGC | |
| GCTTTCTCAT AGCTCACGCT GTAGGTATCT CAGTTCGGTG TAGGTCGTTC GCTCCAAGCT | |
| GGGCTGTGTG CACGAACCCC CCGTTCAGCC CGACCGCTGC GCCTTATCCG GTAACTATCG | |
| TCTTGAGTCC AACCCGGTAA GACACGACTT ATCGCCACTG GCAGCAGCCA CTGGTAACAG | |
| GATTAGCAGA GCGAGGTATG TAGGCGGTGC TACAGAGTTC TTGAAGTGGT GGCCTAACTA | |
| CGGCTACACT AGAAGAACAG TATTTGGTAT CTGCGCTCTG CTGAAGCCAG TTACCTTCGG | |
| AAAAAGAGTT GGTAGCTCTT GATCCGGCAA ACAAACCACC GCTGGTAGCG GTGGTTTTTT | |
| TGTTTGCAAG CAGCAGATTA CGCGCAGAAA AAAAGGATCT CAAGAAGATC CTTTGATCTT | |
| TTCTACGGGG TCTGACGCTC AGTGGAACGA AAACTCACGT TAAGGGATTT TGGTCATGAG | |
| ATTATCAAAA AGGATCTTCA CCTAGATCCT TTTAAATTAA AAATGAAGTT TTAAATCAAT | |
| CTAAAGTATA TATGAGTAAA CTTGGTCTGA CAGTTACCAA TGCTTAATCA GTGAGGCACC | |
| TATCTCAGCG ATCTGTCTAT TTCGTTCATC CATAGTTGCC TGACTCGGGG GGGGGGGGCG | |
| CTGAGGTCTG CCTCGTGAAG AAGGTGTTGC TGACTCATAC CAGGCCTGAA TCGCCCCATC | |
| ATCCAGCCAG AAAGTGAGGG AGCCACGGTT GATGAGAGCT TTGTTGTAGG TGGACCAGTT | |
| GGTGATTTTG AACTTTTGCT TTGCCACGGA ACGGTCTGCG TTGTCGGGAA GATGCGTGAT | |
| CTGATCCTTC AACTCAGCAA AAGTTCGATT TATTCAACAA AGCCGCCGTC CCGTCAAGTC | |
| AGCGTAATGC TCTGCCAGTG TTACAACCAA TTAACCAATT CTGATTAGAA AAACTCATCG | |
| AGCATCAAAT GAAACTGCAA TTTATTCATA TCAGGATTAT CAATACCATA TTTTTGAAAA | |
| AGCCGTTTCT GTAATGAAGG AGAAAACTCA CCGAGGCAGT TCCATAGGAT GGCAAGATCC | |
| TGGTATCGGT CTGCGATTCC GACTCGTCCA ACATCAATAC AACCTATTAA TTTCCCCTCG | |
| TCAAAAATAA GGTTATCAAG TGAGAAATCA CCATGAGTGA CGACTGAATC CGGTGAGAAT | |
| GGCAAAAGCT TATGCATTTC TTTCCAGACT TGTTCAACAG GCCAGCCATT ACGCTCGTCA | |
| TCAAAATCAC TCGCATCAAC CAAACCGTTA TTCATTCGTG ATTGCGCCTG AGCGAGACGA | |
| AATACGCGAT CGCTGTTAAA AGGACAATTA CAAACAGGAA TCGAATGCAA CCGGCGCAGG | |
| AACACTGCCA GCGCATCAAC AATATTTTCA CCTGAATCAG GATATTCTTC TAATACCTGG | |
| AATGCTGTTT TCCCGGGGAT CGCAGTGGTG AGTAACCATG CATCATCAGG AGTACGGATA | |
| AAATGCTTGA TGGTCGGAAG AGGCATAAAT TCCGTCAGCC AGTTTAGTCT GACCATCTCA | |
| TCTGTAACAT CATTGGCAAC GCTACCTTTG CCATGTTTCA GAAACAACTC TGGCGCATCG | |
| GGCTTCCCAT ACAATCGATA GATTGTCGCA CCTGATTGCC CGACATTATC GCGAGCCCAT | |
| TTATACCCAT ATAAATCAGC ATCCATGTTG GAATTTAATC GCGGCCTCGA GCAAGACGTT | |
| TCCCGTTGAA TATGGCTCAT AACACCCCTT GTATTACTGT TTATGTAAGC AGACAGTTTT | |
| ATTGTTCATG ATGATATATT TTTATCTTGT GCAATGTAAC ATCAGAGATT TTGAGACACA | |
| ACGTGGCTTT CCCCCCCCCC CCATTATTGA AGCATTTATC AGGGTTATTG TCTCATGAGC | |
| GGATACATAT TTGAATGTAT TTAGAAAAAT AAACAAATAG GGGTTCCGCG CACATTTCCC | |
| CGAAAAGTGC CACCTGACGT CTAAGAAACC ATTATTATCA TGACATTAAC CTATAAAAAT | |
| AGGCGTATCA CGAGGCCCTT TCGTC. | |

V1R—Vaccine vector V1R was constructed to obtain a minimum-sized vaccine vector without unneeded DNA sequences, which still retained the overall optimized heterologous gene expression characteristics and high plasmid yields that V1J and V1Jns afford. It was determined that (1) regions within the pUC backbone comprising the E. coli origin of replication could be removed without affecting plasmid yield from bacteria; (2) the 3′-region of the kan^{r} gene following the kanamycin open reading frame could be removed if a bacterial terminator was inserted in its place; and, (3) ˜300 bp from the 3′-half of the BGH terminator could be removed without affecting its regulatory function (following the original KpnI restriction enzyme site within the BGH element). V1R was constructed by using PCR to synthesize three segments of DNA from V1Jns representing the CMVintA promoter/BGH terminator, origin of replication, and kanamycin resistance elements, respectively. Restriction enzymes unique for each segment were added to each segment end using the PCR oligomers: SspI and XhoI for CMVintA/BGH; EcoRV and BamHI for the kan^{r} gene; and, BclI and SalI for the oni^{r}. These enzyme sites were chosen because they allow directional ligation of each of the PCR-derived DNA segments with subsequent loss of each site: EcoRV and SspI leave blunt-ended DNAs which are compatible for ligation while BamHI and BclI leave complementary overhangs as do SalI and XhoI. After obtaining these segments by PCR each segment was digested with the appropriate restriction enzymes indicated above and then ligated together in a single reaction mixture containing all three DNA segments. The 5′-end of the oni^{r} was designed to include the T2 rho independent terminator sequence that is normally found in this region so that it could provide termination information for the kanamycin resistance gene. The ligated product was confirmed by restriction enzyme digestion (>8 enzymes) as well as by DNA sequencing of the ligation junctions. DNA plasmid yields and heterologous expression using viral genes within V1R appear similar to V1Jns. The net reduction in vector size achieved was 1346 bp (V1Jns=4.86 kb; V1R=3.52 kb). PCR oligomer sequences used to synthesize V1R (restriction enzyme sites are underlined and identified in brackets following sequence) are as follows: (1) 5′-GGTACAAATATTGGCTATTGG CCATTGCATACG-3′ (SEQ ID NO: 19) [SspI]; (2) 5′-CCACATCTCGAGGAAC CGGGTCAAITTCTTCAGCACC-3′ (SEQ ID NO: 20) [XhoI] (for CMVintA/BGH segment); (3) 5′-GGTACAGATATCGGAAAGCCACGTTGTG TCTCAAAATC-3′ (SEQ ID NO: 21) [EcoRV]; (4) 5′-CACATGGATCCGTAAT GCTCTGCCAGTGTT ACAACC-3′ (SEQ ID NO: 2) [BamHI], (for kanamycin resistance gene segment) (5) 5′-GGTACATG ATCACGTAGAAAAGATCA AAGGATCTTCTTG-3′ (SEQ ID NO: 23) [BclI]; (6) 5′-CCACATGTCGACCCGTAAA AAGGCCGCGITTGCTGG-3′ (SEQ ID NO: 24): [SalI], (for E. coli origin of replication).

The nucleotide sequence of vector V1R is as follows:

| | |
|---|---|
| TCGCGCGTTT CGGTGATGAC GGTGAAAACC TCTGACACAT GCAGCTCCCG GAGACGGTCA | (SEQ ID NO:25) |
| CAGCTTGTCT GTAAGCGGAT GCCGGGAGCA GACAAGCCCG TCAGGGCGCG TCAGCGGGTG | |
| TTGGCGGGTG TCGGGGCTGG CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATGG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACGGGGATTT CCAAGTCTCC ACCCCATTGA | |
| CGTCAATGGG AGTTTGTTTT GGCACCAAAA TCAACGGGAC TTTCCAAAAT GTCGTAACAA | |
| CTCCGCCCCA TTGACGCAAA TGGGCGGTAG GCGTGTACGG TGGGAGGTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGAGTC TATAGGCCCA CCCCCTTGGC | |
| TTCTTATGCA TGCTATACTG TTTTTGGCTT GGGGTCTATA CACCCCCGCT TCCTCATGTT | |
| ATAGGTGATG GTATAGCTTA GCCTATAGGT GTGGGTTATT GACCATTATT GACCACTCCC | |
| CTATTGGTGA CGATACTTTC CATTACTAAT CCATAACATG GCTCTTTGCC ACAACTCTCT | |
| TTATTGGCTA TATGCCAATA CACTGTCCTT CAGAGACTGA CACGGACTCT GTATTTTTAC | |
| AGGATGGGGT CTCATTTATT ATTTACAAAT TCACATATAC AACACCACCG TCCCCAGTGC | |
| CCGCAGTTTT TATTAAACAT AACGTGGGAT CTCCACGCGA ATCTCGGGTA CGTGTTCCGG | |
| ACATGGGCTC TTCTCCGGTA GCGGCGGAGC TTCTACATCC GAGCCCTGCT CCCATGCCTC | |
| CAGCGACTCA TGGTCGCTCG GCAGCTCCTT GCTCCTAACA GTGGAGGCCA GACTTAGGCA | |
| CAGCACGATG CCCACCACCA CCAGTGTGCC GCACAAGGCC GTGGCGGTAG GGTATGTGTC | |
| TGAAAATGAG CTCGGGGAGC GGGCTTGCAC CGCTGACGCA TTTGGAAGAC TTAAGGCAGC | |
| GGCAGAAGAA GATGCAGGCA GCTGAGTTGT TGTGTTCTGA TAAGAGTCAG AGGTAACTCC | |
| CGTTGCGGTG CTGTTAACGG TGGAGGGCAG TGTAGTCTGA GCAGTACTCG TTGCTGCCGC | |
| GCGCGCCACC AGACATAATA GCTGACAGAC TAACAGACTG TTCCTTTCCA TGGGTCTTTT | |
| CTGCAGTCAC CGTCCTTAGA TCTGCTGTGC CTTCTAGTTG CCAGCCATCT GTTGTTTGCC | |
| CCTCCCCCGT GCCTTCCTTG ACCCTGGAAG GTGCCACTCC CACTGTCCTT TCCTAATAAA | |
| ATGAGGAAAT TGCATCGCAT TGTCTGAGTA GGTGTCATTC TATTCTGGGG GGTGGGGTGG | |
| GGCAGCACAG CAAGGGGGAG GATTGGGAAG ACAATAGCAG GCATGCTGGG GATGCGGTGG | |
| GCTCTATGGG TACCCAGGTG CTGAAGAATT GACCCGGTTC CTCCTGGGCC AGAAAGAAGC | |
| AGGCACATCC CCTTCTCTGT GACACACCCT GTCCACGCCC CTGGTTCTTA GTTCCAGCCC | |
| CACTCATAGG ACACTCATAG CTCAGGAGGG CTCCGCCTTC AATCCCACCC GCTAAAGTAC | |
| TTGGAGCGGT CTCTCCCTCC CTCATCAGCC CACCAAACCA AACCTAGCCT CCAAGAGTGG | |
| GAAGAAATTA AAGCAAGATA GGCTATTAAG TGCAGAGGGA GAGAAAATGC CTCCAACATG | |
| TGAGGAAGTA ATGAGAGAAA TCATAGAATT TCTTCCGCTT CCTCGCTCAC TGACTCGCTG | |
| CGCTCGGTCG TTCGGCTGCG GCGAGCGGTA TCAGCTCACT CAAAGGCGGT AATACGGTTA | |
| TCCACAGAAT CAGGGGATAA CGCAGGAAAG AACATGTGAG CAAAAGGCCA GCAAAAGGCC | |
| AGGAACCGTA AAAAGGCCGC GTTGCTGGCG TTTTTCCATA GGCTCCGCCC CCCTGACGAG | |
| CATCACAAAA ATCGACGCTC AAGTCAGAGG TGGCGAAACC CGACAGGACT ATAAAGATAC | |
| CAGGCGTTTC CCCCTGGAAG CTCCCTCGTG CGCTCTCCTG TTCCGACCCT GCCGCTTACC | |
| GGATACCTGT CCGCCTTTCT CCCTTCGGGA AGCGTGGCGC TTTCTCAATG CTCACGCTGT | |
| AGGTATCTCA GTTCGGTGTA GGTCGTTCGC TCCAAGCTGG GCTGTGTGCA CGAACCCCCC | |
| GTTCAGCCCG ACCGCTGCGC CTTATCCGGT AACTATCGTC TTGAGTCCAA CCCGGTAAGA | |
| CACGACTTAT CGCCACTGGC AGCAGCCACT GGTAACAGGA TTAGCAGAGC GAGGTATGTA | |
| GGCGGTGCTA CAGAGTTCTT GAAGTGGTGG CCTAACTACG GCTACACTAG AAGGACAGTA | |
| TTTGGTATCT GCGCTCTGCT GAAGCCAGTT ACCTTCGGAA AAAGAGTTGG TAGCTCTTGA | |
| TCCGGCAAAC AAACCACCGC TGGTAGCGGT GGTTTTTTTG TTTGCAAGCA GCAGATTACG | |
| CGCAGAAAAA AAGGATCTCA AGAAGATCCT TTGATCTTTT CTACGGGGTC TGACGCTCAG | |
| TGGAACGAAA ACTCACGTTA AGGGATTTTG GTCATGAGAT TATCAAAAAG GATCTTCACC | |
| TAGATCCTTT TAAATTAAAA ATGAAGTTTT AAATCAATCT AAAGTATATA TGAGTAAACT | |
| TGGTCTGACA GTTACCAATG CTTAATCAGT GAGGCACCTA TCTCAGCGAT CTGTCTATTT | |
| CGTTCATCCA TAGTTGCCTG ACTCCGGGGG GGGGGGGCGC TGAGGTCTGC CTCGTGAAGA | |
| AGGTGTTGCT GACTCATACC AGGCCTGAAT CGCCCCATCA TCCAGCCAGA AAGTGAGGGA | |
| GCCACGGTTG ATGAGAGCTT TGTTGTAGGT GGACCAGTTG GTGATTTTGA ACTTTTGCTT | |
| TGCCACGGAA CGGTCTGCGT TGTCGGGAAG ATGCGTGATC TGATCCTTCA ACTCAGCAAA | |
| AGTTCGATTT ATTCAACAAA GCCGCCGTCC CGTCAAGTCA GCGTAATGCT CTGCCAGTGT | |
| TACAACCAAT TAACCAATTC TGATTAGAAA AACTCATCGA GCATCAAATG AAACTGCAAT | |
| TTATTCATAT CAGGATTATC AATACCATAT TTTTGAAAAA GCCGTTTCTG TAATGAAGGA | |
| GAAAACTCAC CGAGGCAGTT CCATAGGATG GCAAGATCCT GGTATCGGTC TGCGATTCCG | |
| ACTCGTCCAA CATCAATACA ACCTATTAAT TTCCCCTCGT CAAAAATAAG GTTATCAAGT | |
| GAGAAATCAC CATGAGTGAC GACTGAATCC GGTGAGAATG GCAAAAGCTT ATGCATTTCT | |
| TTCCAGACTT GTTCAACAGG CCAGCCATTA CGCTCGTCAT CAAAATCACT CGCATCAACC | |
| AAACCGTTAT TCATTCGTGA TTGCGCCTGA GCGAGACGAA ATACGCGATC GCTGTTAAAA | |
| GGACAATTAC AAACAGGAAT CGAATGCAAC CGGCGCAGGA ACACTGCCAG CGCATCAACA | |
| ATATTTTCAC CTGAATCAGG ATATTCTTCT AATACCTGGA ATGCTGTTTT CCCGGGGATC | |
| GCAGTGGTGA GTAACCATGC ATCATCAGGA GTACGGATAA AATGCTTGAT GGTCGGAAGA | |
| GGCATAAATT CCGTCAGCCA GTTTAGTCTG ACCATCTCAT CTGTAACATC ATTGGCAACG | |
| CTACCTTTGC CATGTTTCAG AAACAACTCT GGCGCATCGG GCTTCCCATA CAATCGATAG | |
| ATTGTCGCAC CTGATTGCCC GACATTATCG CGAGCCCATT TATACCCATA TAAATCAGCA | |
| TCCATGTTGG AATTTAATCG CGGCCTCGAG CAAGACGTTT CCCGTTGAAT ATGGCTCATA | |
| ACACCCCTTG TATTACTGTT TATGTAAGCA GACAGTTTTA TTGTTCATGA TGATATATTT | |
| TTATCTTGTG CAATGTAACA TCAGAGATTT TGAGACACAA CGTGGCTTTC CCCCCCCCCC | |
| CATTATTGAA GCATTTATCA GGGTTATTGT CTCATGAGCG GATACATATT TGAATGTATT | |
| TAGAAAAATA AACAAATAGG GGTTCCGCGC ACATTTCCCC GAAAAGTGCC ACCTGACGTC | |
| TAAGAAACCA TTATTATCAT GACATTAACC TATAAAAATA GGCGTATCAC GAGGCCCTTT | |
| CGTC. | |

### EXAMPLE 2

Codon Optimized HIV-1 Pol and HIV-1 IA Pol Derivatives as DNA Vector Vaccines Synthesis of WT-optpol and IA-opt-pol Gene—Construction of both genes were conducted by Midland Certified Reagent Company (Midland, Tex.) following established strategies. Ten double stranded oligonucleotides, ranging from 159 to 340 bases long and encompassing the entire pol gene, were synthesized by solid state methods and cloned separately into pUC18. For the wt-pol gene, the fragments are as follows:

| | |
|---|---|
| BglII#1-Ecl136II half site at 282 = | pJS6A1-7 |
| PmlI half site at #285 - Ecl136II half site at #597 = | pJS6B2-5 |
| SspI half site at #600 - Ecl1361II half site at #866 = | pJS6C1-4 |
| SmaI half site at #869 - ApaI #1095 = | pJS6D1-4 |
| ApaI #1095 - KpnI #1296 = | pJS6E1-4 |
| KpnI #1296 - XcmI #1636 = | pJS6F1-5 |
| XcmI #1636 - NsiI #1847 = | pJS6G1-2 |
| NsiI #1847 - BclI half site at #2174 = | pJS6H1-14 |
| BclI half site at #2174 - SacI #2333 = | pJS6I1-2 |
| SacI #2333 - BglII #2577 = | pJS6J1-1 |

EcoRI and HindIII sequences were added upstream of each 5′ end and downstream of each 3′ end, respectively, to allow cloning into the EcoRI-HindIII sites of pUC18.

The next stage of the synthesis was to consolidate these cassettes into three roughly equal fragments (alpha, beta, gamma) and was performed as follows:

Alpha: The SspI-HindIII small fragment of pJS6C1-4 was transferred into the Ecl136II-HindIII sites of pJS6B2-5 to give pJS6BC1-1. Into the EcoRI-PmlI sites of this plasmid was inserted the EcoRI-Ecl136II small fragment of pJS6A1-7 to give pJS6α1-8.

Beta: The EcoRI-ApaI small fragment of pJS6D1-4 was inserted into the corresponding sites of pJS6E1-2 to give pJS6DE1-2. Also, the EcoRI-XcmI small fragment of pJS6F1-5 was inserted into the corresponding sites of pJS6G1-2 to give pJS6FG1-1. Then the EcoRI-KpnI small fragment of pJS6DE1-2 was inserted into the corresponding sites of pJS6FG1-1 to give pJS6β1-1.

Gamma: The SacI-HindIII small fragment of pJS6J1-1 was inserted into the corresponding sites of pJS6I1-2 to give pJS6IJ1-1. This plasmid was propagated through E. coli SCS 110 (dam-/dcm-) to permit subsequent cleavage at the BclI site. The BclI-HindIII small fragment of the unmethylated pJS6IJ1-1 was inserted into the BglII-HindIII sites of pJS6H1-14 to give pJS6χ1-1.

The wt-pol alpha, beta, gamma were ligated into the entire sequence as follows:

The EcoRI-Ecl136II small fragment of pJS6α1-8 was inserted into the EcoRI-SmaI sites of pJS6β1-1 to give pJS6αβ2-1.

Into the NsiI-HindIII sites of this plasmid was inserted the NstI-HindIII small fragment of pJS6χ1-1 to give pUC18-wt-pol. This final plasmid was completely resequenced in both strands.

To construct the entire IA-pol gene, only 3 new small fragments were synthesized:

| | |
|---|---|
| PmlI half site at #285 - Ecl136II half site at #597 = | pJS7B1-1 |
| KpnI #1296 - XmI #1636 = | pJS7F1-2 |
| NsiI #1847 - BglII half site at #2174 = | pJS7H1-5 |

These were then used in the same reconstruction strategy as described above to give pUC18-IA-pol.

Expression Vector Construction—pUC18-wt-pol and pUC18-IA-pol were digested with BglII in order to isolate fragments containing the entire pol genes. V1R, V1Jns, V1Jns-tpa (Shiver, et al., 1995, Immune responses to HIV gp120 elicited by DNA vaccination. In Vaccines 95 (eds. Chanock, R. M., Brown, F., Ginsberg, H. S., & Norrby, E.) @ pp. 95-98; Cold Spring Harbor Laboratory Press, Cold Spring Harbor, New York; see also Example Section 1) were digested with BglII. The cut vectors were then treated with calf intestinal alkaline phosphatase. Both wt-pol and IA-pol genes were ligated into cut V1R using T4 DNA ligase (16° C., overnight). Competent DH5α cells were transformed with aliquots of the ligation mixtures. Colonies were screened by restriction digestion of amplified plasmid isolates. Following a similar strategy, the BglII fragment containing the IA-pol was subcloned into the BglII site of V1Jns. To ligate the IA-pol gene into V1Jns-tpa, the IA-pol gene was PCR-amplified from V1R-IA-pol using pfu polymerase and the following pair of primers: 5′-GGTACAAGATCTCCGCCCCCATCTCCCCCATTGAGA-3′ (SEQ ID NO: 26), and 5′-CCACATAGATCTGCCCGGGCTTTAGTCCTCATC-3′ (SEQ ID NO: 27). The upstream primer was designed to remove the initiation met codon and place the pol gene in frame with the tpa leader coding sequence from V1Jns-tpa The PCR product was purified from the agarose gel slab using Sigma DNA Purification spin columns. The purified products were digested with BglII and subcloned into the BglII site of V1Jns-tpa.

Results—The codon humanized wt- and IA-pol genes were constructed via stepwise ligation of 10 synthetic dsDNA fragments (Ferretti, et al., 1986, Proc. Natl. Acad. Sci. USA 83: 599-603). For expression in mammalian systems, the IA-pol gene was subcloned into V1R, V1Jns, and V1Jns-tpa. All these vectors place the gene under the control of the human cytomegalovirus/intron A hybrid promoter (hCMVIA). The DNA sequence of the IA-pol gene and the expressed protein product are shown in FIGS. 2A-B. Subcloning into V1Jns-tpa attaches the leader sequence from human tissue-specific plasminogen activator (tpa) to the N-terminus of the IA-pol (Pennica, et al., 1983, Nature 301: 214-221) to allow secretion of the protein. The sequences of the tpa leader and the fusion junction are shown in FIG. 3.

### EXAMPLE 3

HIV-1 POL Vaccine—Rodent Studies

Materials—E. coli DH5α strain, penicillin, streptomycin, ACK lysis buffer, hepes, L-glutamine, RPMI1640, and ultrapure CsCl were obtained from Gibco/BRL (Grand Island, N.Y.). Fetal bovine serum (FBS) was purchased from Hyclone. Kanamycin, Tween 20, bovine serum albumin, hydrogen peroxide (30%), concentrated sulfuric acid, β-mercaptoethanol (β-ME ), and concanavalin A were obtained from Sigma (St. Louis, Mo.). Female balb/c mice at 4-6 wks of age were obtained from Taconic Farms (Germantown, N.Y.). 0.3-mL insulin syringes were purchased from Myoderm. 96-well flat bottomed Maxisorp plates were obtained form NUNC (Rochester, N.Y.). HIV-1_{IIIB} RT p66 recombinant protein was obtained from Advanced Biotechnologies, Inc. (Columbia, Md.). 20-mer peptides were synthesized by Research Genetics (Huntsville, Ala.). Horseradish peroxidase (HRP)-conjugated rabbit anti-mouse IgG1 was obtained from ZYMED (San Francisco, Calif.). 1,2-phenylenediamine dihydrochloride (OPD) tablets was obtained from DAKO (Norway). Purified rat anti-mouse IFN-gamma (IgG1, clone R4-6A2), biotin-conjugated rat anti-mouse IFN-gamma (IgG1, clone XMG 1.2), and strepavidin-alkaline phosphatase conjugate were purchased from PharMingen (San Diego, Calif.). 1-STEP NBT/BCIP dye was obtained from Pierce Chemicals (Rockford, Ill.). 96-well Multiscreen membrane plate was purchased from Millipore (France). Cell strainer was obtained from Becton-Dickinson (Franklin Lakes, N.J.).

Plasmid Preparation—E. coli DH5α cells expressing the pol plasmids were grown to saturation in LB broth supplemented with 100 ug/mL kanamycin. Plasmid were purified by standard CsCl method and solubilized in saline at concentrations greater than 5 mg/mL until further use.

Vaccination—The plasmids were prepared in phosphate-buffered saline and administered into balb/c by needle injection (28-½G insulin syringe) of 50 uL aliquot into each quad muscle. V1Jns-IApol was administered at 0.3, 3, 30 ug dose and for comparison, V1Jns-tpa-IApol was given at 30 ug dose. Immunizations were conducted at T=0 and T=8 wks (for select animals from the 30-ug dose cohorts).

ELISA Assay—At T=12 wks, blood samples were collected by making an incision of a tail vein and the serum separated. Anti-RT titers were obtained following standard secondary antibody-based ELISA. Briefly, Maxisorp plates were coated by overnight incubation with 100 uL of 1 ug/mL HIV-1 RT protein (in PBS). The plates were washed with PBS/0.05% Tween 20 and incubated for approx. 2 h with 200 ul/well of blocking solution (PBS/0.05% tween/1% BSA). The blocking solution was decanted; 100 uL aliquot of serially diluted serum samples were added per well and incubated for 2 h at room temperature. The plates were washed and 100 uL of 1/1000-diluted HRP-rabbit anti-mouse IgG were added with 1 h incubation. The plates were washed thoroughly and soaked with 100 uL OPD/H₂O₂ solution for 15 min. The reaction was quenched by adding 100 uL of 0.5M H₂SO4 per well. OD₄₉₂ readings were recorded.

ELIspot—Spleens were collected from 5 mice/cohort at T=13-14 wks and pooled into a tube of 8-mL R10 medium (RPMI1640, 10% FBS, 2 mM L-glutamine, 100 U/mL Penicillin, 100 u/mL streptomycin, 10 mM Hepes, 50 uM β-ME). Multiscreen opaque plates were coated with 100 μl/well of capture mAb (purified R4-6A2 diluted in PBS to 5 μg/ml) at 4° C. overnight. The plates were washed with PBS/Pen/Strep in hood and blocked with 200 μl/well of complete R10 medium for 37° C. for at least 2 hrs. The mouse spleens were ground on steel mesh, collected into 15 ml tubes and centrifuged at 1200 rpm for 10 min. The pellet was treated in ACK buffer (4 ml of lysis buffer per spleen) for 5 min at room temperature to lyse red blood cells. The cell pellet was centrifuged as before, resuspended in K-medium (5 ml per mouse spleen), filtered through a cell strainer and counted using a hemacytometer. Block medium was decanted from the plates and 100 μl/well of cell samples (5.0×10e5 cells per well) plus antigens were added. Pol-specific CD4⁺ cells were stimulated using a mixture of previously identified two epitope-containing peptides (aa641-660, aa731-750). Antigen-specific CD8+ cells were stimulated using a pool of four peptide epitope-containing peptides (aa201-220, aa311-330, aa571-590, aa781-800) or with individual peptides. A final concentration of 4 ug/mL per peptide was used. Each splenocyte sample is tested for IFN-gamma secretion by adding the mitogen, concanavalin A. Plates were incubated at 37° C., 5% CO₂ for 20-24 h. The plates were washed with PBS/0.05% Tween 20 and soaked with 100 uL/well of 5 ug/mL biotin-conjugated rat anti-mouse IFN-mAb (clone XMG1.2) at 4° C. overnight. The plates were washed and soaked with 100 uL/well 1/2500 dilution of strepavidin-AP (in PBS/0.005% Tween/5% FCS) for 30 min at 37° C. Following a wash, spots were developed by incubating with 100 μl/well 1-step NBT/BCIP for 6-10 min. The plates were washed with water and allowed to air dry. The number of spots in each wells were determined using a dissecting microscope and normalized to 10e6 cells.

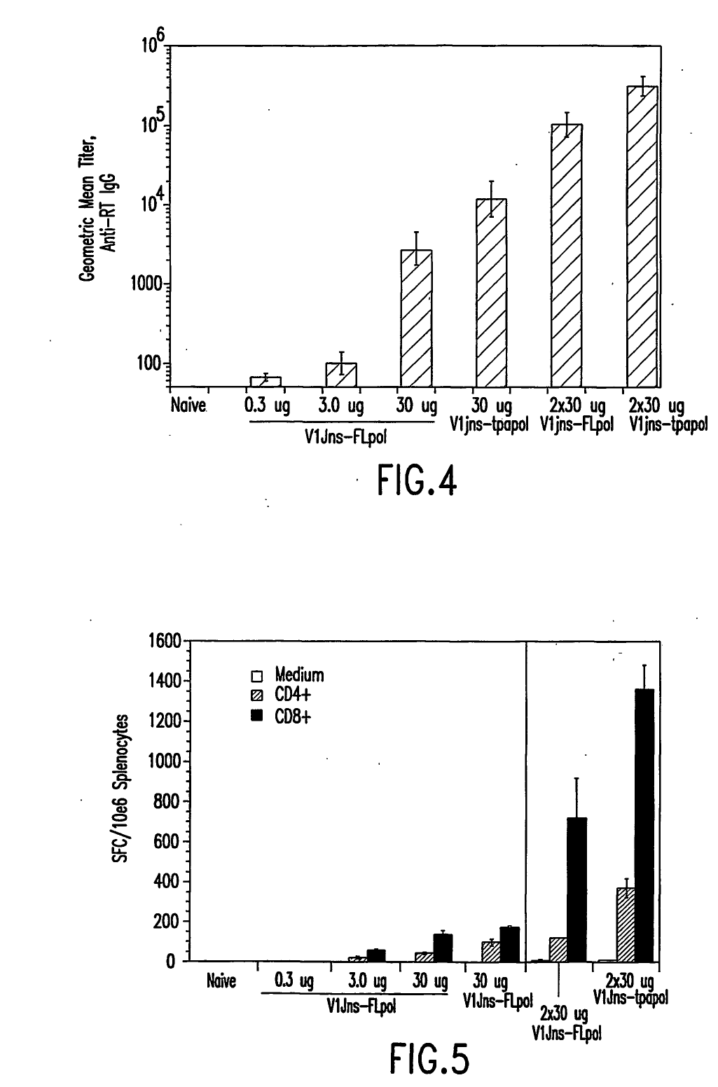

Results—Single vaccination of balb/c mice with V1Jns-IApol is able to induce antigen-specific antibody (FIG. 4) and T cell (FIG. 5) responses in a dose response manner. IFN-gamma secretion from splenocytes can be detected from 3 and 30 ug cohort following stimulation with pools of peptides that contain CD4+ and CD8+ T cell epitopes. These epitopes were identified by (1) screening 20-mer peptides that encompass the entire pol sequence and overlap by 10 amino acid for ability to stimulate IFN-gamma secretion from vaccinee splenocytes, and (2) determining the T cell type (CD4+ or CD8+) by depleting either population in an Elispot assay. Addition of tpa leader sequence to the pol gene is able to induce comparable, if not slightly higher, frequencies of pol-specific CD4+ and CD8+ cells. A second immunization with either V1Jns-IApol and V1Jns-tpa-IApol resulted in effective boosting of the immune responses.

### EXAMPLE 4

HIV-1 Pol Vaccine—Non Human Primate Studies

Materials—E. coli DH5α strain, penicillin, streptomycin, and ultrapure CsCl were obtained from Gibco/BRL (Grand Island, N.Y.). Kanamycin and phytohemagluttinin (PHA-M) were obtained from Sigma (St. Louis, Mo.). 20-mer peptides were synthesized by SynPep (Dublin, Calif.) and Research Genetics (Huntsville, Ala.). 96-well Multiscreen Immobilon-P membrane plates were obtained from Millipore (France). Strepavidin-alkaline phosphatase conjugate were purchased form Pharmingen (San Diego, Calif.). 1-Step NBT/BCIP dye was obtained form Pierce Chemicals (Rockford, Ill.). Rat anti-human IFN-gamma mAb and biotin-conjugated anti-human IFN-gamma reagent were obtained from R&D Systems (Minneapolis, Minn.). Dynabeads M450 anti-human CD4 were obtained from Dynal (Norway). HIVp24 antigen assay was purchased from Coulter Corporation (Miami, Fla.). HIV-1_{IIIB} RT p66 recombinant protein was obtained from Advanced Biotechnologies, Inc. (Columbia, Md.). Plastic 8 well strips/plates, flat bottom, Maxisorp, are obtained from NUNC (Rochester, N.Y.). HIV+ human serum 9711234 was obtained from Biological Specialty Corp.

Plasmid Preparation—E. coli DH5α cells expressing the pol plasmids were grown to saturation in LB supplemented with 100 ug/mL kanamycin. Plasmid were purified by standard CsCl method and solubilized in saline at concentrations greater than 5 mg/mL until further use.

Vaccination—Cohorts of 3 rhesus macaques (approx. 5-10 kg) were vaccinated with 5 mg dose of either V1Jns-IApol or V1Jns-tpa-IApol. The vaccine was administered by needle injection of two 0.5 mL aliquots of 5 mg/mL plasmid solution (in phosphate-buffered saline, pH 7.2) into both deltoid muscles. Prior to vaccination, the monkeys were chemically restraint with i.m. injection of 10 mg/kg ketamine. The animals were immunized 3× at 4 week intervals (T=0, 4, 8.wks).

Sample Collection—Blood samples were collected at T=0, 4, 8, 12, 16, 18 wks; sera and PBMCs were isolated using established protocols.

ELIspot Assay—Immobilon-IP plates were coated with 100 uL/well of rat anti-human IFN-gamma mAb at 15 ug/mL at 4° C. overnight. The plates are then washed with PBS and block by adding 200 uL/well of R10 medium. 4×10e5 peripheral blood cells were plated per well and to each well, either media or one of the pol peptide pools (final concentration of 4 ug/mL per peptide) or PHA, a known mitogen, is added to a final volume of 100 uL. Duplicate wells were set up per sample per antigen and stimulation was performed for 20-24 h at 37° C. The plates are then washed; biotinylated anti-human IFN-gamma reagent is added (0.1 ug/mL, 100 uL per well) and allowed to incubate for overnight at 4° C. The plates are again washed and 100 uL of 1:2500 dilution of the strepavidin-alkaline phosphatase reagent (in PBS/0.005% Tween/5% FCS) is added and allowed to incubate for 2 h at ambient room temperature. After another wash, spots are developed by incubating with 100 uL/well of 1-step NBT/BCIP for 6-10 min. CD4− T cell depletion was performed by adding 1 bead particle/10 cell of Dynabeads M450 anti-human CD4, prewashed with PBS, and incubating on the shaker at 4° C. for 30 min. The beads are fractionated magnetically and the unbound cells collected and quantified before plating onto the ELISpot assay plates ( at 4×10e5 cells per well).

CTL Assay— Procedures for establishing bulk CTL culture with fresh or cryopreserved peripheral blood mononuclear cells (PBMC) are as follows. Twenty percent total PBMC were infected in 0.5 ml volume with recombinant vaccinia virus, Vac-tpaPol, respectively, at multiplicity of infection (moi) of 5 for 1 hr at 37° C., and then combined with the remaining PBMC sample. The cells were washed once in 10 ml R-10 medium, and plated in a 12 well plate at approximately 5 to 10×10⁶ cells/well in 4 ml R-10 medium. Recombinant human IL-7 was added to the culture at the concentration of 330 U/ml. Two or three days later, one milliliter of R-10 containing recombinant human IL-2 (100 U/ml) was added to each well. And twice weekly thereafter, two milliliters of cultured media were replaced with 2 ml fresh R-10 medium with rhIL2 (100 U/ml). The lymphocytes were cultured at 37° C. in the presence of 5% CO₂ for approximately 2 weeks, and used in cytotoxicity assay as described below. The effector cells harvested from bulk CTL cultures were tested against autologous B lymphoid cell lines (BLCL) sensitized with peptide pools. To. prepare for the peptide-sensitized targets, the BLCL cells were washed once with R-10 medium, enumerated, and pulsed with peptide pool (about 4 to 8 μg/ml concentration for each individual peptide) in 1 ml volume overnight. A mock target was prepared by pulsing cells with peptide-free DMSO diluent to match the DMSO concentration in the peptide-pulsed targets. The cells were enumerated the next morning, and 1×10⁶ cells were resuspended in 0.5 ml R-10 medium. Five to ten microliters of Na⁵¹CrO₄ were added to the tubes at the same time, and the cells were incubated for 1 to 2 hr 37° C. The cells were then washed 3 times and resuspended at 5×10⁴ cells/ml in R-10 medium to be used as target cells. The cultured lymphocytes were plated with target cells at designated effector to target (E:T) ratios in triplicates in 96-well plates, and incubated at 37° C. for 4 hours in the presence of 5% CO₂. A sample of 30 μl supernatant from each well of cell mixture was harvested onto a well of a Lumaplate-96 (Packard Instrument, Meriden, Conn.), and the plate was allowed to air dry overnight. The amount of ⁵¹Cr in the well was determined through beta-particle emission, using a plate counter from Packard Instrument. The percentage of specific lysis was calculated using the formula as: % specific lysis =(E-S)/(M-S). The symbol E represents the average cpm released from target cells in the presence of effector cells, S is the spontaneous cpm released in the presence of medium only, and M is the maximum cpm released in the presence of 2% Triton X-100.

ELISA Assay—The pol-specific antibodies in the monkeys were measured in a competitive RT EIA assay, wherein sample activity is determined by the ability to block RT antigen from binding to coating antibody on the plate well. Briefly, Maxisorp plates were coated with saturating amounts of pol positive human serum (97111234). 250 uL of each sample is incubated with 15 uL of 266 ng/mL RT recombinant protein (in RCM 563, 1% BSA, 0.1% tween, 0.1% NaN₃) and 20 uL of lysis buffer (Coulter p24 antigen assay kit) for 15 min at room temperature. Similar mixtures are prepared using serially diluted samples of a standard and a negative control which defines maximum RT binding. 200 uL/well of each sample and standard were added to the washed plate and the plate incubated 16-24 h at room temperature. Bound RT is quantified following the procedures described in Coulter p24 assay kit and reported in milliMerck units per mL arbitrarily defined by the chosen standard.

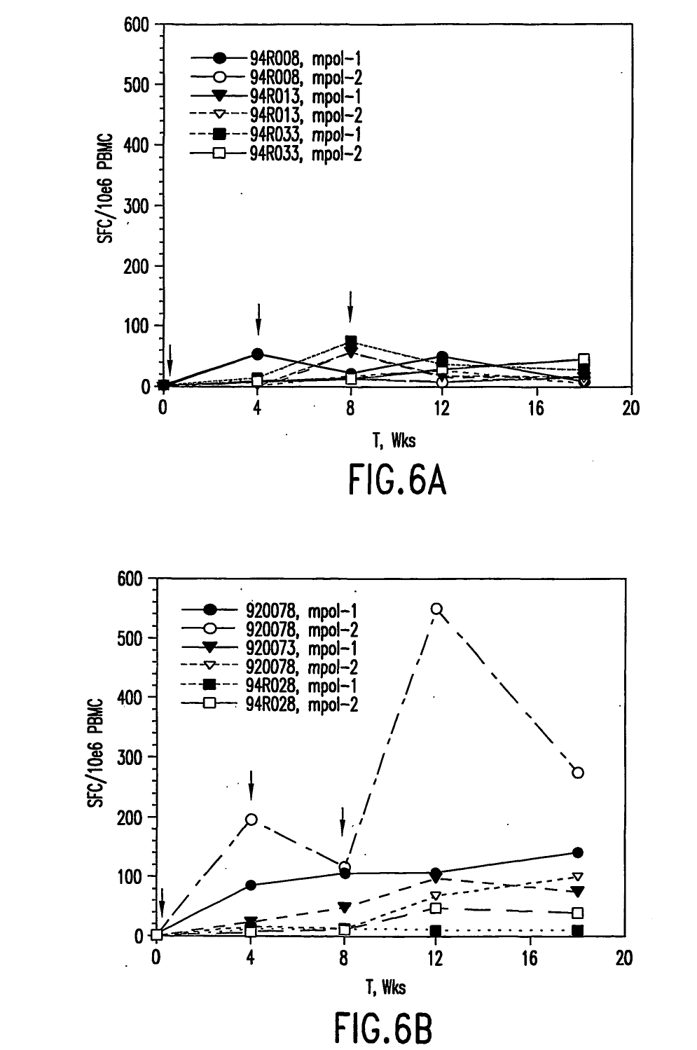

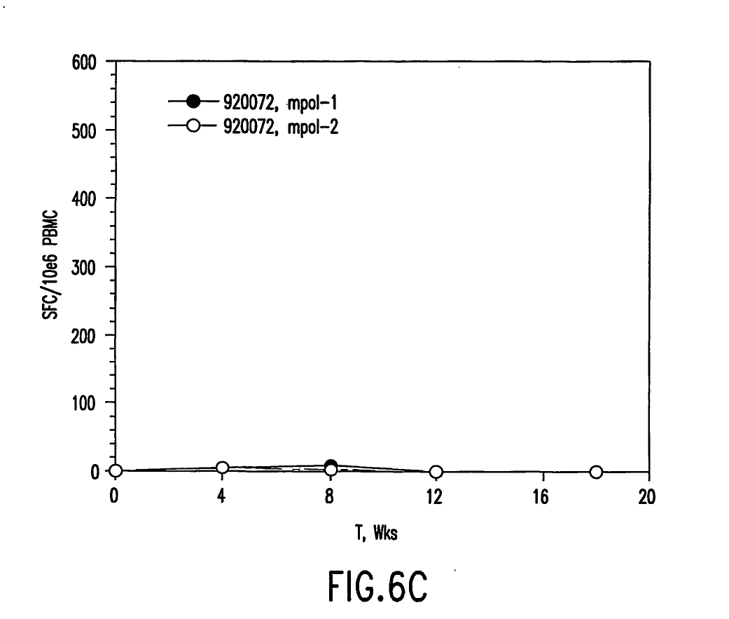

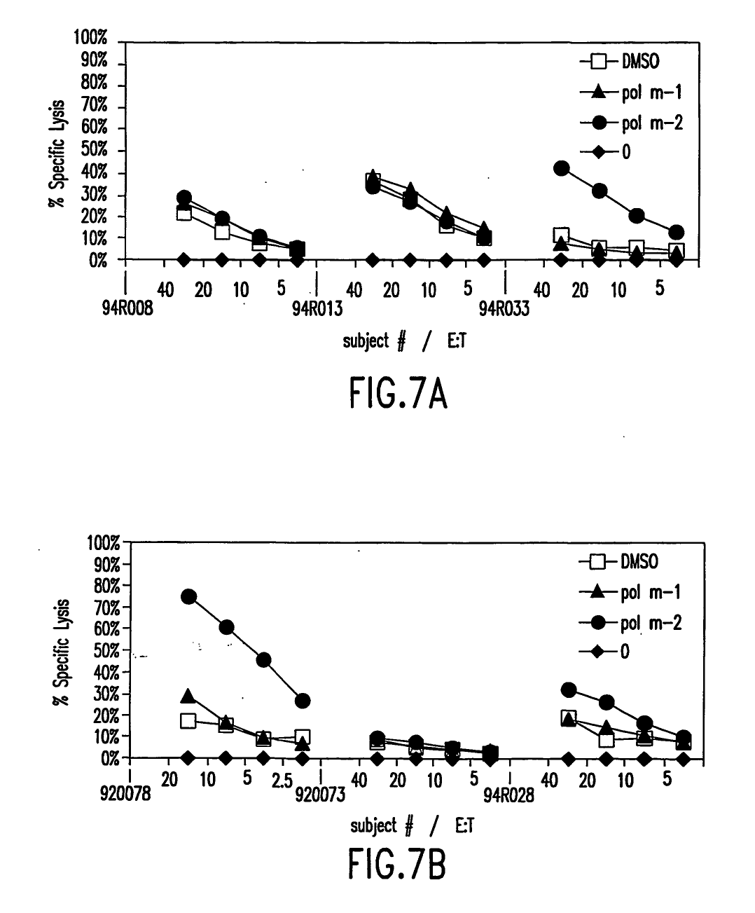

Results—Repeated vaccinations with V1Jns-IApol induced in 1 of 3 monkeys (94R033) significant levels of antigen-specific T cell activation (FIGS. 6A-C and Table 2) and CTL killing of peptide-pulsed autologous cells (FIGS. 7A-B). A significant CD8+ component to the T cell responses in this animal was confirmed by peptide-stimulation of CD4depleted PBMCs in an ELIspot assay (Table 2).

Immunization with V1Jns-tpa-IApol produced T cell responses from all 3 vaccinees (FIGS. 6A-C, FIGS. 7A-B; Table 2). Two (920078, 94R028) exhibited bulk CTL activity and detectable CD8+ components as measured by Elispot analyses of CD4-depleted PBMCs. For the third monkey (920073), the activated T cells were largely CD4+ (Table 2). Table 3 shows the time course data on the frequency of IFN-gamma secreting cells (SFC/million cells) upon antigen-specific stimulation for monkeys vaccinated 3× with either V1Jns-IApol or V1Jns-tpa-IApol (5 mg dose). At T=18 wks, CD4-cell depletion were performed; the reported values are the number of spots per million of fractionated cells and are not corrected for the resultant enrichment of CD8+ T cells. PBMCs were stimulated with peptide pools that represent either IA pol protein (mpol-1, mpol-2) or wt Pol (wtpol-1, wtpol-2).

TABLE 2

| | | | T = 0 wk | T = 4 Wk | T = 8 Wk | | T = 18 Wk | |
|---|---|---|---|---|---|---|---|---|
| Vaccine | Animal No. | Antigen | Dose 1 | Dose 2 | Dose 3 | T = 12 Wk | | CD4-Depi |
| VIJns-IApd | 94R008 | medum | 1 | 15 | 6 | 11 | 11 | 11 |
| 5 mgs | | mpd-1 | 3 | 69 | 28 | 61 | 20 | 15 |
| | | mpd-2 | 0 | 25 | 21 | 19 | 28 | 16 |
| | | wtpd-1 | | 49 | 20 | 53 | 18 | |
| | | wtpd-2 | | 34 | 24 | 24 | 19 | |
| | 94R013 | medum | 0 | 14 | 6 | 9 | 18 | 11 |
| | | mpd-1 | 0 | 9 | 63 | 25 | 34 | 9 |
| | | mpd-2 | 1 | 15 | 24 | 36 | 24 | 15 |
| | | wtpd-1 | | 9 | 50 | 33 | 18 | |
| | | wtpd-2 | | 6 | 21 | 29 | 25 | |
| | 94R033 | medum | 4 | 15 | 11 | 14 | 13 | 8 |
| | | mpd-1 | 3 | 29 | 86 | 51 | 41 | 24 |
| | | mpd-2 | 0 | 24 | 25 | 43 | 59 | 64 |
| | | wtpd-1 | | 30 | 38 | 60 | 53 | |
| | | wtpd-2 | | 48 | 46 | 86 | 61 | |
| VIJns-tpa-IApd | 920078 | medum | 0 | 24 | 13 | 11 | 14 | 11 |
| 5 mgs | | mpd-1 | 3 | 110 | 120 | 119 | 155 | 11 |
| | | mpd-2 | 1 | 221 | 130 | 561 | 289 | 145 |
| | | wtpd-1 | | 115 | 53 | 70 | 116 | |
| | | wtpd-2 | | 218 | 204 | 490 | 194 | |
| | 920073 | medum | 0 | 13 | 3 | 15 | 15 | 6 |
| | | mpd-1 | 0 | 36 | 51 | 113 | 90 | 14 |
| | | mpd-2 | 0 | 29 | 16 | 83 | 115 | 34 |
| | | wtpd-1 | | 20 | 35 | 100 | 74 | |
| | | wtpd-2 | | 25 | 16 | 79 | 61 | |
| | 94R028 | medum | 0 | 18 | 11 | 18 | 19 | 9 |
| | | mpd-1 | 1 | 30 | 24 | 29 | 30 | 28 |
| | | mpd-2 | 1 | 24 | 23 | 66 | 59 | 95 |
| | | wtpd-1 | | 23 | 25 | 34 | 29 | |
| | | wtpd-2 | | 26 | 28 | 71 | 43 | |
| Näe | 920072 | medum | 1 | 19 | 3 | 38 | 9 4 | |
| | | mpd-1 | 0 | 24 | 11 | 25 | 4 | 6 |
| | | mpd-2 | 1 | 24 | 5 | 28 | 6 | 5 |
| | | wtpd-1 | | 18 | 13 | 20 | 6 | |
| | | wtpd-2 | | 23 | 14 | 33 | 14 | |

For the Elispot assay, antigen specific stimulation were performed by using pools of 20-mer peptide pools based on the vaccine sequence. The vaccine pol sequence differs from the wild-type HIV-1 sequence by 9 point mutations, thereby affecting 16 of the 20-mer peptides in the pool. Comparable responses were observed in the vaccinees when these peptides are replaced with those using the wild-type sequences.

Four of the vaccinees gave anti-RT titers above background after 3 dosages of the plasmids (Table 2).

TABLE 3
Anti-RT levels in Rhesus Macaques Vaccinated 3x (4 week intervals) with
5 mgs of V1Jns-IApol or V1Jns-tpa-IApol expressed in mMU/mL.

| | T = 0 Wk | T = 4 | T = 8 | | |
|---|---|---|---|---|---|
| Vaccine/Monkey | Dose 1 | Dose 2 | Dose 3 | T = 12 | T = 16 |
| V1Jns-IApol, 5 mg | | | | | |
| 94R008 | ND | <10 | <10 | 15 | 14 |
| 94R013 | ND | <10 | <10 | <10 | <10 |
| 94R033 | ND | <10 | <10 | 25 | 19 |
| V1Jns-tpa-IApol, 5 mg | | | | | |
| 920078 | ND | <10 | <10 | 35 | 17 |
| 920073 | ND | <10 | <10 | <10 | <10 |
| 94R028 | ND | <10 | <10 | 20 | 63 |

### EXAMPLE 5

Effect of Codon Optimization on In Vivo Expression and Cellular Immune Response of wt-pol

Materials and Methods—Extraction of virus-derived pol gene—The gene for RT-IN (wt-pol; a non-codon optimized wild type pol gene derived directly from the HIV IIIB genome) was extracted and amplified from the HIV IIIB genome using two primers, 5′-CAG GCG AGA TCT ACC ATG GCC CCC ATT AGC CCT ATT GAG ACT GTA-3′ (SEQ ID NO: 29) and 5′-CAG GCG AGA TCT GCC CGG GCT TTA ATC CTC ATC CTG TCT ACT TGC CAC-3′ (SEQ ID NO: 30 ), containing BglII sites. The reaction contained 200 nmol of each primer, 2.5 U of pfu Turbo DNA polymerase (Stratagene, La Jolla, Calif.), 0.2 mM of each dNTPs, and the template DNA in 10 mM KCl, 10 mM (NH₄)₂SO₄, 20 mM Tris-HCl pH 8.75, 2mM MgSO₄, 0.1% TritonX-100, 0.1 mg/ml bovine serum albumin (BSA). Thermocycling conditions were as follows: 20 cycles of 1 min at 95° C., 1 min at 56° C., and 4 mins at 72° C. with 15-min capping at 72° C. The digested PCR fragment was subcloned into the BglII site of the expression plasmid V1Jns (Shiver, et al., 1995, Immune responses to HIV gp120 elicited by DNA vaccination. In Chanock, R. M., Brown, F., Ginsberg, H. S., and Norrby, E. (Eds.) Vaccines 95. Cold Spring Harbor Laboratory Press, Cold Spring Harbor, N.Y., pp 95-98; see also Example section 1 herein) expression plasmid following similar procedures as described above. The ligation mixtures were then used to transform competent E. coli DH5 cells and screened by PCR amplification of individual colonies. Sequence of the entire gene insert was confirmed. All plasmid constructs for animal immunization were purified by CsCl method (Sambrook, et al., 1989, Fritsch and Maniatis, T. (Eds) Molecular cloning: a laboratory manual. Cold Spring Harbor Laboratory Press, Cold Spring Harbor).

In vitro expression in mammalian cells—1.5×10⁶ 293 cells were transfected with 1 or 10 μg of V1R-wt-pol (codon optimized) and V1Jns-wt-pol (virus derived) using the Cell Phect kit and incubated for 48 h at 37° C., 5% CO₂, 90% humidity. Supernatants and cell lysates were prepared and assayed for protein content using Pierce Protein Assay reagent (Rockford, Ill.). Aliquots containing equal amounts of total protein were loaded unto 10-20% Tris glycine gel (Novex, San Diego, Calif.) along with the appropriate molecular weight markers. The pol product was detected using anti-serum from a seropositive patient (Scripps Clinic, San Diego, Calif.) diluted 1:1000 and the bands developed using goat anti-human IgG-HRP (Bethyl, Montgomery, Tex.) at 1:2000 dilution and standard ECL reagent kit (Pharmacia LKB Biotechnology, Uppsala, Sweden).

Ultrasensitive RT activity assay of pol constructs—RT activities from codon optimized wt-pol and IA pol plasmids were analyzed by the Product-Enhanced Reverse Transcriptase (PERT) assay using Perkin Elmer 7700, Taqman technology (Arnold, et al., 1999, One-step fluorescent probe product-enhanced reverse transcriptase assay. In McClelland, M., Pardee, A. (Eds.) Expression genetics: accelerated and high-throughput methods. Biotechniques Books, Natick, Mass., pp. 201-210). Background levels for this assay were determined using 1:100,000 dilution of lysates from mock (chemical treatment only, no vector) transfected 293 cells. This background range is set as RT/reaction tube of 0.00 to 56.28 which is taken from the mean value of 13.80±3 standard deviations (sd=14.16). Any individual value>56.28 would be considered positive for PERT assay. Cells lysates were prepared similarly for the following samples: mock transfection with empty V1Jns vector; no vector control; transfection with V1Jns-tpa-pol (codon optimized); and transfection with V1Jns-IApol (codon optimized). Samples were serially diluted to 1:100,000 in PERT buffer and 24 replicates for each sample at this dilution were assayed for RT activity.

Rodent immunization with optimized and virus-derived pol plasmids—To compare the immunogenic properties of wt-pol (codon optimized) and virus-derived pol gene, cohorts of BALB/c mice (N=10) were vaccinated with 1 μg, 10 μg, and 100 μg doses of V1R-wt-pol (codon optimized) and V1Jns-wt-pol plasmid (virus derived). At 5 weeks post dose 1, 5 of 10 mice per cohort were boosted with the same dose of plasmid they initially received. In all cases, the vaccines were suspended or diluted in 6 mM sodium phosphate, 150 mM sodium chloride, pH 7.2, and the total dose was injected to both quadricep muscles in 50 μL aliquots using a 0.3-mL insulin syringe with28-½G needles (Becton-Dickinson, Franklin Lakes, N.J.).

Anti-RT ELISA—Anti-RT titers were obtained following standard secondary antibody-based ELISA. Maxisorp plates (NUNC, Rochester, N.Y.) were coated by overnight incubation with 100 μL of 1 μg/mL HIV-1 RT protein (Advanced Biotechnologies, Columbia, Md.) in PBS. The plates were washed with PBS/0.05% Tween 20 using Titertek MAP instrument (Hunstville, Ala.) and incubated for approximately 2 h with 200 μL/well of blocking solution (PBS/0.05% tween/1% BSA). The blocking solution was decanted; 100 μL aliquot of serially diluted serum samples were added per well and incubated for 2 h at room temperature. An initial dilution of 100-fold is performed followed by 4-fold serial dilution. The plates were washed and 100 μL of 1/1000-diluted HRP-rabbit anti-mouse IgG (ZYMED, San Francisco, Calif.) were added with 1 h incubation. The plates were washed thoroughly and soaked with 100 μL 1,2-phenylenediamine dihydrochloride/hydrogen peroxide (DAKO, Norway) solution for 15 min. The reaction was quenched by adding 100 μL of 0.5M H₂SO4 per well. OD₄₉₂ readings were recorded using Titertek Multiskan MCC/340 with S20 stacker. Endpoint titers were defined as the highest serum dilution that resulted in an absorbance value of greater than or equal to 0.1 OD₄₉₂ (2.5 times the background value).

ELIspot assay—Antigen-specific INFγ-secreting cells from mouse spleens were detected using the ELIspot assay (Miyahira, et al., 1995, Quantification of antigen specific CD8⁺ T cells using an ELISPOT assay. J. Immunol. Methods 1995, 181, 45-54). Typically, spleens were collected from 3-5 mice/cohort and pooled into a tube of 8-mL complete RPMI media (RPMI1640, 10% FBS, 2 mM L-glutamine, 100 U/mL Penicillin, 100 u/mL streptomycin, 10 mM Hepes, 50 uM β-ME). Multiscreen opaque plates (Millipore, France) were coated with 100 μL/well of 5 μg/mL purified rat anti-mouse IFN-γ IgG1, clone R4-6A2 (Pharmingen, San Diego, Calif.), in PBS at 4° C. overnight. The plates were washed with PBS/penicillin/streptomycin in hood and blocked with 200 μL/well of complete RPMI media for 37° C. for at least 2 h. The mouse spleens were ground on steel mesh, collected into 15 ml tubes and centrifuged at 1200 rpm for 10 min. The pellet was treated with 4 mL ACK buffer (Gibco/BRL) for 5 min at room temperature to lyse red blood cells. The cell pellet was centrifuged as before, resuspended in complete RPMI media (5 ml per mouse spleen), filtered through a cell strainer and counted using a hemacytometer. Block media was decanted from the plates and to each well, 100 μL of cell samples (5×10⁵ cells per well) and 100 μL of the antigen solution were added. To the control well, 100 μL of the media were added; for specific responses, peptide pools containing either CD4⁺ or CD8⁺ epitopes were added. In all cases, a final concentration of 4 μg/mL per peptide was used. Each sample/antigen mixture were performed in triplicate wells. Plates were incubated at 37° C., 5% CO₂, 90% humidity for 20-24 h. The plates were washed with PBS/0.05% Tween 20 and incubated with 100 μL/well of 1.25 μg/mL biotin-conjugated rat anti-mouse IFN-γ mAb, clone XMG1.2 (Pharmingen) at 4° C. overnight. The plates were washed and incubated with 100 μL/well 1/2500 dilution of strepavidin-alkaline phosphatase conjugate (Pharmingen) in PBS/0.005% Tween/5% FBS for 30 min at 37° C. Following a wash, spots were developed by incubating with 100 μl/well 1-step NBT/BCIP (Pierce Chemicals) for 6-10 min. The plates were washed with water and allowed to air dry. The number of spots in each well was determined using a dissecting microscope and the data normalized to 10⁶ cell input.

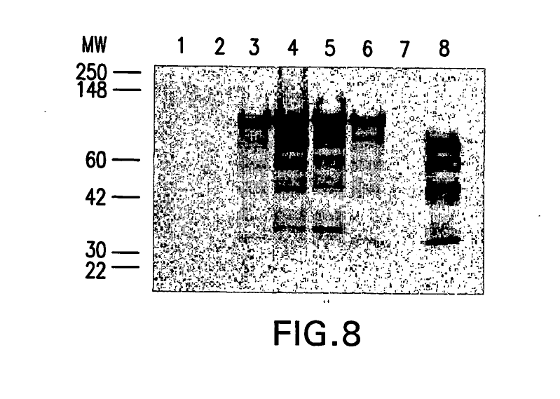

Results—In vitro expression of pol in mammalian cells—Heterologous expression of the optimized wt or IA pol genes (V1R-wt-pol (codon optimized), V1Jns-IApol (codon optimized), V1Jns-tpa-IApol (codon optimized)) in 293 cells (FIG. 8) yielded a single polypeptide of correct approximate molecular size (90-kDa) for the RT-IN fusion product. In contrast, no expression could be detected by transfecting cells with 1 and 10 μg of the V1Jns-wt-pol, which bears the virus-derived pol.

Ultrasensitive RT assay of cells transfected with Pol constructs—Table 4 summarizes the levels of polymerase activity from mock (vector only) control, IApol (codon optimized)and wt-pol plasmids (codon optimized). Results indicate that the wild-type POL transfected cells contained RT activity approximately 4-5 logs higher than the 293 cell only baseline values. Mock transfected cells contained activity no higher than baseline values. The RT activity from opt-IApol-transfected cells was also found to be no different than baseline values; no individual reaction tube resulted in RT activity higher than the established cut-off value of 56.

TABLE 4

| | Avg. | | | |
|---|---|---|---|---|
| Sample | RT/tube | Standard deviation | Minimum | Maximum |
| Vector only | 16.25 | 18.52 | 0.0 | 42.99 |
| IApol (codon | 2.99 | 8.01 | 0.0 | 35.20 |
| optimized) | | | | |
| Wt-pol | 126147 | 21338 | 68973 | 152007 |
| (codon | | | | |
| optimized) | | | | |

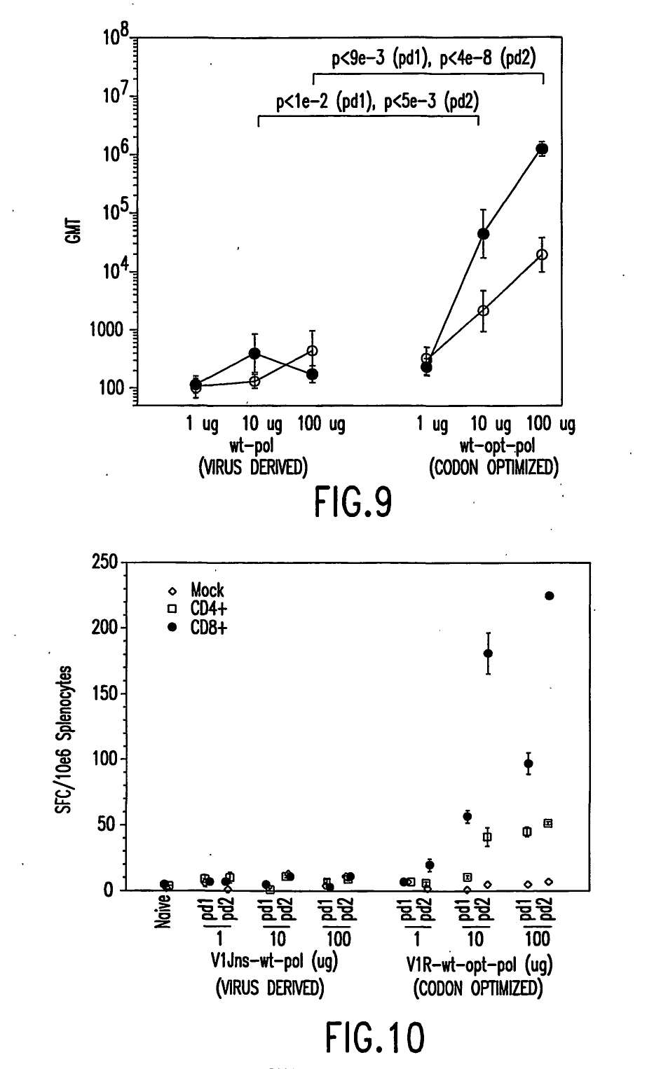

Comparative immunogenicity of optimized and virus-derived pol plasmid—To compare the in vivo potencies of both constructs, BALB/c mice (N=10 per group) were vaccinated with escalating doses (1, 10, 100 μg) of either V1Jns-wt-pol (virus derived) or V1R-wt-pol (codon optimized). At 5 wks post dose 1, 5 of 10 animals were randomly boosted with the same vaccine and dose they received initially. FIG. 9 shows the geometric mean titers of the BALB/c cohorts determined at 2 wks past boost. No significant anti-RT titers can be observed from animals immunized with one or two doses of the wt-pol plasmid (virus derived). In contrast, animals vaccinated with the humanized gene construct gave cohort anti-RT titers (>1000) significantly above background levels at doses above 10 ug. The responses seen at 10 and 100 ug dose of V1R-wt-pol (codon optimized) were boosted approximately 10-fold with a second immunization, reaching titers as high as 10⁶.

Spleens from all mice in each of the cohorts were collected to be analyzed for IFN-γ secretion following stimulation with mixtures of either CD4+ peptide epitopes or CD8+ peptide epitopes. The results are shown in FIG. 10. All wt-pol vaccinees did not show any significant cellular response above the background controls. In contrast, strong antigen-stimulated IFN-γ secretion were observed in a dose-responsive manner from animals vaccinated with one or two doses of 10 or more μg of the wt-pol (codon optimized) construct.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

What is claimed is:

1. A pharmaceutically acceptable DNA vaccine composition, which comprises:
(a) a DNA expression vector; and,
(b) a DNA molecule containing a codon optimized open reading frame encoding a Pol protein or inactivated Pol derivative thereof,
wherein upon administration of the DNA vaccine to a host the Pol protein or inactivated Pol derivative is expressed and generates a cellular immune response against HIV-1 infection.

2. The DNA vaccine of claim 1 wherein the DNA molecule encodes wild type Pol.

3. The DNA vaccine of claim 2 wherein the DNA molecule comprises the nucleotide sequence as set forth in SEQ ID NO: 1.

4. The DNA vaccine of claim 3 which is V1Jns-wt-pol.

5. The DNA vaccine of claim 1 wherein the DNA molecule encodes an inactivated Pol derivative which contains a nucleotide sequence encoding a human tissue plasminogen activator leader peptide.

6. The DNA vaccine of claim 5 wherein the DNA molecule comprises the nucleotide sequence as set forth in SEQ ID NO: 5

7. The DNA vaccine of claim 6 which is V1Jns-tPA-wt-pol.

8. The DNA vaccine of claim 1 wherein the inactivated Pol protein contains at least one amino acid modification within each region of the Pol protein responsible for reverse transcriptase activity, RNase H activity and integrase activity, such that the inactivated Pol protein shows no substantial reverse transcriptase activity, RNase H activity and integrase activity.

9. The DNA vaccine of claim 8 wherein the DNA molecule comprises the nucleotide sequence as set forth in SEQ ID NO: 3

10. The DNA vaccine of claim 9 which is V1Jns-IAPol.

11. The DNA vaccine of claim 8 wherein the DNA molecule encodes an inactivated Pol derivative which contains a nucleotide sequence encoding a human tissue plasminogen activator leader peptide.

12. The DNA vaccine of claim 11 wherein the DNA molecule comprises the nucleotide sequence as set forth in SEQ ID NO: 7.

13. The DNA vaccine of claim 7 which is V1Jns-tPA-IAPol.

14. A method for inducing an immune response against infection or disease caused by virulent strains of HIV which comprises administering into the tissue of a mammalian host a pharmaceutically acceptable DNA vaccine composition which comprises a DNA expression vector and a DNA molecule containing a codon optimized open reading frame encoding a Pol protein or inactivated Pol derivative thereof, wherein upon administration of the DNA vaccine to the vertebrate host the Pol protein or inactivated Pol derivative is expressed and generates the immune response.

15. The method of claim 16 wherein the mammalian host is a human.

16. The method of claim 17 wherein the DNA vaccine is selected from the group consisting of V1Jns-WTPol, V1Jns-tPA-WTPol, V1Jns-IAPol and V1Jns-tPA-IAPol.

17. A substantially purified protein which comprises an amino acid sequence selected from the group consisting of SEQ ID NO: 4, SEQ ID NO: 6, and SEQ ID NO: 8.
